(19) United States
(12) Patent Application Publication
(10) Pub. No.: US 2024/0240177 A1
(43) Pub. Date: Jul. 18, 2024

(54) MULTITARGETING RNA COMPOSITIONS

(71) Applicant: SYSTEMS ONCOLOGY, LLC, Scottsdale, AZ (US)

(72) Inventors: Spyro Mousses, Scotsdale, AZ (US); David Azorsa, Scotsdale, AZ (US); Daniel Feldheim, Boulder, CO (US); James Heil, Boulder, CO (US); Necky Tran, London, Ontario (CA); Gregory Allen Penner, Toronto, Ontario (CA)

(21) Appl. No.: 18/289,514

(22) PCT Filed: May 5, 2022
(86) PCT No.: PCT/US2022/027932

§ 371 Date: Nov. 3, 2023

Related U.S. Application Data
(60) Provisional application No. 63/185,359, filed on May 6, 2021.
(60) Provisional application No. 63/231,234, filed on Aug. 9, 2021.
(60) Provisional application No. 63/242,865, filed on Sep. 10, 2021.
(60) Provisional application No. 63/250,548, filed on Sep. 30, 2021.
(60) Provisional application No. 63/287,037, filed on Dec. 7, 2021.
(60) Provisional application No. 63/287,040, filed on Dec. 7, 2021.
(60) Provisional application No. 63/323,997, filed on Mar. 25, 2022.

(51) Int. Cl. C12N 15/113; A61K 31/713; A61P 35/00; C12N 15/115

(52) U.S. Cl. CPC C12N 15/113; A61K 31/713; A61P 35/00; C12N 15/115; C12N 2310/14; C12N 2310/16; C12N 2310/3519; C12N 2310/51

(57) ABSTRACT

The invention provides siRNA compositions for inhibiting gene expression in targeted cells.

### RELATED AND PRIORITY APPLICATIONS

This application is a National Stage of International Application No. PCT/US2022/027932, filed May 5, 2022, which claims priority to U.S. Provisional Patent Application No. 63/185,359 filed May 6, 2021, U.S. Provisional Patent Application No. 63/231,234 filed Aug. 9, 2021, U.S. Provisional Patent Application No. 63/242,865 filed Sep. 10, 2021, U.S. Provisional Patent Application No. 63/250,548 filed Sep. 30, 2021, U.S. Provisional Patent Application No. 63/287,037 filed Dec. 7, 2021, U.S. Provisional Patent Application No. 63/287,040 filed Dec. 7, 2021 and U.S. Provisional Patent Application No. 63/323,997 filed Mar. 25, 2022. All of the above applications are incorporated herein by reference in their entirety.

### FIELD OF THE INVENTION

The invention is generally directed to siRNA compositions for inhibiting gene expression in targeted cells.

### SEQUENCE LISTING

The instant application contains a Sequence Listing which has been submitted electronically in ASCII format and is hereby incorporated by reference in its entirety. Said ASCII copy, created on May 26, 2022, is named 10911_011094-WO0_SL.txt and is 110,450 bytes in size.

### BACKGROUND OF THE INVENTION

RNA interference (RNAi), also known as RNA silencing, has been extensively explored for therapeutic use inB reducing gene expression but in the decades since its discovery few therapeutics have been approved. The traditional design pattern for RNA inhibition is that one piece of siRNA aims at one specific sequence (Reynolds et al., Nat Biotechnol, 22:326-330 (2004)). There remains a need in the art for compositions and methods of delivering siRNA to cells (e.g., malignant cells, tumor-associated T cells, effector T cells) to inhibit diseases such as cancer, metastasis or metabolic diseases. The nucleic acid compounds and methods of using the same as provided herein solve these and other problems in the art.

Recent work has expanded the RNA constructs to include joining two siRNAs to inhibit two different targets (Liu et al., Sci Reports, 6: (2016)). SiRNA's processed by cellular RNAi machinery to produce two siRNAs as opposed to dual administration offers a number of benefits including increased circulating half-life and reduced renal excretion (Liu et al., Sci Reports, 6: (2016)).

Dual targeting of genes by a single siRNA through targeting conserved homologous regions has been shown to be effective to inhibit the expression of gene families by diminishing the function of escape pathways. In vitro, a multi-target siRNA targeting the conserved homology region of DNMT3 family members effectively inhibited expression (Du et al., Gen and Mol Bio, 35:164-171(2012)).

Delivery to tissues other than the liver has remained a complication and hinderance for RNAi therapies. Aptamer-siRNA chimeras have been used to effectively deliver siRNAs to downregulate expression of oncological genes targets (Liu et al., Sci Reports, 6: (2016)).

U.S. Pat. No. 6,506,559 discloses a method to inhibit expression of a target gene in a cell, the method comprising the introduction of a double-stranded RNA into the cell in an amount sufficient to inhibit expression of the target gene, wherein the RNA is a double-stranded molecule with a first ribonucleic acid strand consisting essentially of a ribonucleotide sequence which corresponds to a nucleotide sequence of the target gene and a second ribonucleic acid strand consisting essentially of a ribonucleotide sequence which is complementary to the nucleotide sequence of the target gene. Furthermore, the first and the second ribonucleotide strands are separately complementary strands that hybridize to each other to form the said double-stranded construct, and the double-stranded construct inhibits expression of the target gene.

U.S. Pat. No. 5,475,096 discloses nucleic acid molecules each having a unique sequence, each of which has the property of binding specifically to a desired target compound or molecule. Each nucleic acid molecule is a specific ligand of a given target compound or molecule. The process, known as SELEX, is based on the idea that nucleic acids have sufficient capacity to form a variety of two- and three-dimensional structures with sufficient chemical versatility available within their monomers to act as ligands (form specific binding pairs) with virtually any chemical compound, whether monomeric or polymeric. Molecules of potentially any size can serve as targets.

The SELEX method involves selection from a mixture of candidates and step-wise iterations of structural improvement, using the same general selection theme, to achieve virtually any desired criterion of binding affinity and selectivity. Starting from a mixture of nucleic acids, preferably comprising a segment of randomized sequence, the method includes steps of contacting the mixture with the target under conditions favorable for binding, partitioning unbound nucleic acids from those nucleic acids which have bound to target molecules, dissociating the nucleic acid-target pairs, amplifying the nucleic acids dissociated from the nucleic acid-target pairs to yield a ligand-enriched mixture of nucleic acids, then reiterating the steps of binding, partitioning, dissociating and amplifying through as many cycles as desired.

U.S. Pat. No. 9,953,131 discloses a method for designing a dual-targeting short interfering RNAs (siRNAs) in which both strands are deliberately designed to separately target different mRNA transcripts with complete complementarity. This approach is limited by the rarity of useful sequences of this type.

U.S. Pat. No. 9,777,278 discloses an interfering nucleic acid (iNA) duplex comprised of a sense strand of nucleotides having a 5′ end and a 3′ end annealed onto an antisense strand of nucleotides having a 5′ end and a 3′ end wherein the antisense strand has at least two segments, wherein one segment of the antisense strand can target a first RNA and another segment of the antisense strand can target a second RNA, or one segment of the antisense strand can target a first portion of an RNA and another segment of the antisense strand can target a second non-contiguous portion of said RNA.

U.S. Pat. No. 9,695,425 discloses an siRNA molecule that, when internalized by a B cell, suppresses expression of BAFF-R and one other target oncogene selected from: Bcl6, Bcl2, STAT3, Cyclin D1, Cyclin E2 and c-myc.

U.S. Pat. No. 10,689,654 discloses a bivalent siRNA-aptamer chimera capable of silencing two or more genes. Methods of using the bivalent siRNA chimeras for selectively targeting cells to down-regulate the expression of multiple genes is also disclosed (incorporated herein in its entirety by reference).

Du et al., Gen and Mol Bio, 35:164-171(2012) discloses a siRNA targeting the conserved homologous region of DNMT3 family members.

U.S. Pat. No. 10,689,654 discloses a bivalent siRNA-aptamer chimera platform that incorporates two aptamers for increase efficiency of delivering siRNAs to the targeted cell. Furthermore, those aptamers are conjugated to an siRNA construct that is processed by cellular RNAi machinery to produce at least two different siRNAs to inhibit expression of two or more different genes (incorporated herein in its entirety by reference).

U.S. patent application Ser. No. 15/899,473 discloses bispecific aptamers (incorporated herein in its entirety by reference).

U.S. Pat. No. 9,567,586 discloses an EPCAM aptamer coupled to an siRNA.

U.S. Pat. No. 10,385,343 discloses a method of treating cancer by administering a chimeric molecule comprising an EPCAM binding aptamer domain and an inhibitory nucleic acid domain that targets Plk1.

Patent Application PCT/US2020/038355 discloses an EpCAM-binding aptamer domain conjugated to an siRNA that inhibits the expression of a gene selected from the group consisting of: UPF2; PARP1; APE1; PD-L1; MCL1; PTPN2; SMG1; TREX1; CMAS; and CD47 for the purpose of treating cancer.

U.S. Pat. No. 10,960,086 discloses an siRNA-aptamer chimera that utilizes two aptamers targeting HER2 and HER3 and an siRNA targeting EGFR (incorporated herein in its entirety by reference).

U.S. Pat. No. 8,828,956 N-acetylgalactosamine (GalNAc)-siRNA conjugates that enables subcutaneous dosing of RNAi therapeutics with potent and durable effects and a wide therapeutic index. This delivery systems is only effective for delivering to the liver as GalNAc binds to the Asialoglycoprotein receptor (ASGPR) that is predominantly expressed on liver hepatocytes.

U.S. Pat. No. 8,058,069 discloses lipid nanoparticle (LNP) delivery technology. LNP technology (formerly referred to as stable nucleic acid-lipid particles or SNALP) encapsulates siRNAs with high efficiency in uniform lipid nanoparticles that are claimed to be effective in delivering RNAi therapeutics to disease sites in various preclinical models.

U.S. Pat. No. 10,278,986 discloses an antibody conjugated to an siRNA as a delivery mechanism. The antibody targets C5aR and the siRNA targets C5 expression for the treatment of rheumatoid arthritis. Patent Application PCT/US2020/036307 discloses a method of preparing an antibody covalently linked to one or more oligonucleotides.

Aptamers are single-stranded RNA or DNA oligonucleotides that are capable of binding with high affinity and specificity and are cost effective to produce. Aptamers are of great interest as an antibody-like replacement and are being investigate for their ability to selectively bind to a specific target, including proteins, peptides, carbohydrates, etc., as well as function as a ligand for directed drug delivery. However, there are two primary hurdles for aptamers reaching clinical significance, their need to be stabilized for in vivo use against nuclease degradation which results in a short half-life, and their rapid renal clearance due to their small size.

Native DNA aptamers are more stable than RNA aptamers as RNA is a transient messenger. The in vitro half-life of an RNA aptamer in plasma is a few seconds, while a DNA aptamer has a half-life of up to hour (2000 White et al, 2002 Takei et al, 1991 Shaw et al). The 2′ hydroxyl group of RNA makes it chemically unstable, susceptible to hydrolysis, and allows for the catalysis of RNA strand scission by endoribonucleases (2009 Houseley et al). For these reasons, RNA aptamers are commonly chemically modified primarily at the 2′-position of pyrimidines to enhance stability.

U.S. Pat. No. 5,660,985 describes oligonucleotides containing nucleotide derivatives chemically modified at the 5- and 2′-positions of pyrimidines and purines including 2′-fluoro and 2′-amino modifications.

U.S. Pat. No. 5,580,737, describes highly specific nucleic acid ligands containing one or more nucleotides modified with 2′-amino (2′-NH_{#}), 2′-fluoro (2′-F), and/or 2′-O-methyl (2′-OMe).

U.S. patent application Ser. No. 08/264,029 describes oligonucleotides containing various 2′-modified pyrimidines.

U.S. patent application Ser. No. 10/524,817 describes a 4′-thioribonucleotide modified aptamer which was later developed in Kato, Y. et al. (2005) into an aptamer against human α-thrombin.

U.S. Pat. No. 9,914,914 describes six different modifications where the canonical ribofuranose ring of DNA and RNA is replaced by five- or six-membered congeners comprising HNA (1,5 anhydrohexitol nucleic acids), CeNA (cyclohexenyl nucleic acids), LNA (2′-O,4′-C-methylene-β-D-ribonucleic acids; locked nucleic acids), ANA (arabinonucleic acids), FANA (2′-fluoro-arabinonucleic acid) and TNA (a-L-threofuranosyl nucleic acids).

U.S. patent application Ser. No. 61/748,834 describes Threose nucleic acid (TNA) modified aptamers.

U.S. patent application Ser. No. 12/044,895 describes double-stranded locked nucleic acid modifications (2′-O,4′-C-methylene-β-D-ribonucleic acids).

Lato, S. M. (2002) Nucleic Acids Res., describes Ribonucleoside 5′-(alpha-P-borano)-triphosphates (BH₃-RNA) modified aptamers.

PCT Publication No. 1997/004726 describes spiegelmers which are mirror images of the natural aptamers in which the D-ribose (the natural ribose) are replaced with the unnatural L-ribose. PCT Publication NO. 2001/006014 describes one of the first SELEX generated spiegelmers developed against D-adenosine.

Jhaveri, S. et al. (1998) Bioorg. Med. Chem. Lett., describes Ribonucleoside 5′-(alpha-thio) triphosphates (S-RNA) modified aptamers.

PCT Publication NO. 1994/010562 describes RNA aptamers containing photoreactive chromophore 5-iodouridine using crosslinking SELEX.

Immune checkpoint inhibition in cancer therapy has been shown to be effective for the treatment of a number of different types of cancer. However, not all cancers cells respond equally. Additionally, toxicity and the development of resistance to individual checkpoint inhibitors are problematic (Pardoll, 2012; Topalian et al., 2015). Improvements for immune checkpoint inhibitors are needed to combat aforementioned drawbacks.

The first immune-checkpoint inhibitor approved by the U.S. Food and Drug Administration (FDA) was ipilimumab, a fully human immunoglobulin G1 monoclonal antibody that blocks cytotoxic T-lymphocyte antigen (CTLA)-4 and consequently the PD-1 pathway for the treatment of metastatic melanoma in 2011. The finding that programmed cell death protein 1 ligand 1 (PDL1 or PD-L1) and PDL2 are expressed by melanoma cells, T cells, B cells and natural killer cells led to the development of programmed cell death protein 1 (PD1 or PD-1)-specific antibodies (e.g., nivolumab and pembrolizumab).

Thus, PD1 pathway blockade has become a major focus in anticancer drug development beyond melanoma. In addition to benefiting patients with renal cell carcinoma, it has benefit in patients with tumors previously not considered sensitive to immunotherapies, including non-small cell lung cancer. However, there are still limitations due to toxicity associated with these immunotherapies. Thus, there is a need for an immunotherapy blocking the PD1 pathway with the best balance of high efficacy and low toxicity.

Metastatic melanoma is an aggressive disease with a 16% 5-year survival rate and responds poorly to most standard chemotherapies. Interferon and interleukin 2 (IL-2) have both been approved by the U.S. Food and Drug Administration for the treatment of melanoma. Both mediate their benefit by stimulating an antitumor immune response.

Therefore, it is an object of the invention to provide immunotherapies that have reduced off-target effects for the treatment of cancer including but not limited to, ovarian cancer, non-small-cell lung cancer, cervical cancer, prostate cancer, esophageal and stomach cancers, preferably breast cancer.

Lung cancer is a malignant lung tumor characterized by uncontrolled cell growth in tissues of the lung. Lung carcinomas derive from transformed, malignant cells that originate as epithelial cells, or from tissues composed of epithelial cells. In 2020, lung cancer occurred in 2.2 million people globally and caused 1.8 million deaths. It is the most common cause of cancer-related death in men and the second-most common in women after breast cancer. In the United States, five-year survival rate is 20.5%. See https://en.wikipedia.org/wiki/Lung_cancer accessed Sep. 9, 2021. Ninety one percent of lung cancer patients express TROP2 or HER3.

### Her3

The four receptor tyrosine kinase (RTK) family members comprise the Epidermal Growth Factor Receptor (EGFR) family of proteins, including EGFR/ErbB1, HER2/ErbB2, HER3/ErbB3, and HER4/ErbB4. Each member shares the following structural similarities: an extracellular domain (ECD), a single-span transmembrane (TM) domain, and an intracellular domain (ICD) that contains a conserved catalytic kinase domain and carboxy terminal tail. Except for Human epidermal growth factor 2 (HER2), which has no known ligand, ligand binding of EGFR members causes receptor dimerization and transphosphorylation of the ICD, which propagates signaling events through pathways that included PI3K/AKT, MAPK, and JAK/STAT. Activation of these pathways is required for the regulation of cell survival, proliferation, and other functions. Without a ligand, HER2 forms heterodimers with other family members, and high HER2 expression levels may drive homodimerization that supports receptor activation. Aberrant signal initiation and transduction of HER2, HER3, and other family member proteins are associated with multiple cancers. Notably, in breast cancer, HER2 is the dominant RTK and is amplified in approximately 15%-25% of cases. Currently there are numerous agency-approved HER2 inhibitors for the treatment of HER2-positive breast cancers that included small molecule inhibitors, monoclonal antibodies, and antibody-drug conjugates. For example, traztuzumab, an anti-HER2 antibody, is standard of care for HER-2 positive, early-stage breast cancer. HER2-targeted approaches have improved patient outcomes; however, the onset of drug resistance remains a concern. Additionally, previous clinical studies have shown that co-expression of HER2 and HER3 in breast cancer patients correlates with higher rates of relapse, specifically to tamoxifen. Watanabe S, Yonesaka K, Tanizaki J, et al. Targeting of the HER2/HER3 signaling axis overcomes ligand-mediated resistance to trastuzumab in HER2-positive breast cancer. Cancer Med-us. 2019; 8(3):1258-1268. doi:10.1002

Breast cancer characterized by HER2 overexpression correlates with poor prognosis, high aggressiveness, and extensive drug resistance (Slamon, et al., Science, 235:177-182 (1987)). Trastuzumab (Herceptin®), a humanized antibody, is the first line treatment for patients with HER2 positive breast cancer. However, the majority of patients eventually develop resistance.

The resistance to HER2 targeted therapies is associated with the increased levels of other HER family receptors (Narayan, et al., Cancer Res, 69: 2191-2194 (2009)). The HER family consists of EGFR (HER1), HER2, HER3 and HER4 (Arteaga, et al., Cancer Cell, 25: 282-303 (2014)). It is well known that the HER family is interdependent and displays functional redundancy in that the blockade of one HER receptor can often be compensated by another HER family member (Olayioye, et al., EMBO, 19: 3159-3167 (2000)). EGFR, HER2 and HER3 contribute to the initiation and progression of many cancers, and are well recognized therapeutic targets.

Ligand binding to HER receptors results in the formation of homo- and hetero-dimers. EGFR is one of the most studied. HER-family receptors and a key oncogenic driver in many epithelial cancers (Nicholson, et al., Eur J Cancer, 37: S9-S15 (2001)). Accumulating evidence shows that HER3 is a key node in many cancers and involved in the resistance against EGFR- and HER2-targeted therapies through activating a compensatory PI3K-AKT survival pathway (Lee-Hoeflich, et al., Cancer Res, 68: 5878-5887 (2008); Holbro, et al., Proc Natl Acad Sci USA, 100: 8933-8938 (2003)). Resistance to trastuzumab in breast cancer cell lines is associated with upregulation of EGFR and HER3 (Narayan, et al., Cancer Res, 69: 2191-2194. (2009); Ritter, et al., Clin Cancer Res, 13: 4909-4919 (2007)).

HER2/HER3 heterodimer has been identified to function like an oncogene to support the proliferation of HER2-overexpressing tumor cells (Holbro, et al., Proc Natl Acad Sci USA, 100: 8933-8938 (2003)). The cross-talk and compensatory functionalities of HER family receptors provide strong rationales for co-targeting of EGFR, HER2 and HER3 in HER-dependent cancer treatment. In line with the notion of combining treatment, hi-specific antibodies and pan-HER antibodies have been developed and show more efficacious than single receptor targeting antibodies. Bispecific MM11 antibody targeting both HER2 and HER3 is able to suppress the proliferation of HER2-expressing tumor cells (Kontermann, et al., Drug Discov Today, 20:838-847 (2015)). Pan-HER with six antibody mixture targeting EGFR, HER2 and HER3 has been developed and displayed superior potency compared to agents targeting single receptors in preclinical studies (Jacobsen, et al., Clin Cancer Res, 21: 4110-4122 (2015)).

Since HER3 lacks intrinsic kinase activity, TKIs (tyrosine-kinase inhibitors) will be not effective in inhibiting HER3. Antibody combinations are the current strategies to inhibit HER family signaling network (Hu, et al., Cancer Res, 75: 159-170 (2015); lida, et al., Mol Cancer Ther, 15: 2175-2186 (2016)). However, most antibodies are expensive to produce and have high immunogenicity. To address immunogenicity and high cost of antibody, a nucleic acid-based multiple function molecule with a siRNA and two RNA aptamers was engineered. The new siRNA-aptamer chimera is able to target EGFR/HER2/HER3 simultaneously with low toxicity. Recently, aptamer-siRNA chimera, employing only RNA molecules, is attractive for cell type-specific gene silencing. Aptamers are ssDNA or RNA and selected through in vitro enrichment process (Ellington, et al., Nature, 346: 818-822 (1990)). Like antibodies, aptamers can bind to target with high affinity and specificity. Due to small and oligonucleotide properties, aptamers offer many advantages over antibodies including non-immunogenicity, high tissue penetration, thermostability, low cost, and ease of synthesis and modification (Keefe, et al., Nat Rev Drug Discov, 9:537-550 (2010); Zhou et al., Nat Drug Rev Discov, 16: 181-202 (2017)). Current cell type-specific RNA aptamers have been used for targeting delivery of siRNA and drugs (Wheeler, et al., J Clin Inves, 121: 2401-2412 (2011); Neff, et al., Sci Transl Med.)

U.S. Pat. No. 10,960,086 (incorporated herein in its entirety by reference) discloses a HER3 aptamer that is able to specifically bind to the extracellular domains of HER3 (Chen, et al., Proc Natl Acad Sci USA, 100: 9226-9231 (2003)). HER3 aptamer inhibits HRG-dependent tyrosine phosphorylation of HER2 (Takahashi, et al., Sci Rep, 6: 33697 (2016)). With a similar generation approach, HER2 aptamer has been identified and synthesized. HER2 aptamer shows high specificity to HER2 positive but not negative cancer cell lines (Kim, et al., Nucleic Acid Ther, 21:173-178 (2011)).

### TROP2

The glycoprotein, Trophoblast cell surface antigen 2 (Trop2) is expressed on the surface membrane of epithelial cells. In addition to supporting essential roles in placental and embryonic development, stem cell proliferation and organ development, another physiological function of Trop2 involves the regulation of intracellular calcium signaling. Trop2 activity has also been shown to play a role in tumorigenesis through interactions with several cell signaling pathways. For example, increased intracellular calcium concentration activates MAPK, thereby augmenting levels of phosphorylated ERK1 and ERK2, which are mediators of cell cycle progression, angiogenesis, cell proliferation, cell invasion, and metastasis. Trop2 expression is also involved in other pro-oncogenic signaling pathways that regulate cell cycle progression through the activation of cyclin E and D. Moreover, it has been reported that interactions between β-catenin and the intracellular domain of Trop2 enhances stem cell-like, self-renewal properties of cancer cells. Trop2 overexpression is associated with a wide array of human epithelial cancers affecting tissues of the breast, bladder, stomach, colon, pancreas, prostate, cervix, ovary, and other tissues. Recently, the antibody-drug conjugate (ADC), Sacituzumab govitecan received approval from the Food and Drug Administration (FDA) for the treatment of adults with metastatic triple-negative breast cancer (mTNBC). The novel ADC is a conjugation of the irinotecan metabolite, SN-38 (govitecan) and a human monoclonal antibody against Trop2. The FDA has also granted Sacituzumab govitecan fast tract status for other indications, including non-small cell lung cancer (NSCLC) and small-cell lung cancer (SCLC). According to data from The Cancer Genome Atlas (TCGA), approximately 64% of lung cancer patients express Trop2.

The Human epidermal growth factor 3 (HER3) is a mechanism of resistance to EGFR-targeted therapies in lung cancer and other tumor types and has been correlated with poor prognosis in lung cancer and is expressed in 27% of lung cancer patients. To date, no HER3 inhibitors have been agency approved, though several investigational agents are being evaluated in clinical trials of varying stages. For example, Patritumab deruxtecan is a HER3-targeting antibody-drug conjugate that has demonstrated encouraging results in a phase I study of patients with EGFR-mutant non-small cell lung cancer.

Embodiments of the instant invention comprising RNA aptamer-siRNA therapeutics to target Trop2 and HER3 and deliver siRNA that exploit tumor vulnerabilities have the potential to provide an new class of drugs for the treatment of lung cancer and potentially other solid tumors. Syed Y Y. Sacituzumab Govitecan: First Approval. Drugs. 2020; 80(10): 1019-1025. Romaniello D et al., Cancers 2020. Janne P A et al. Journal of Clinical Oncology 2021.

### PSMA

In addition to Prostate-specific antigen (PSA) which is a marker for prostate cancer, Protein-specific membrane antigen (PSMA) is a transmembrane protein that is highly expressed on the surface of prostate cancer cells and is correlated with aggressive prostate cancers and inversely correlated with survival. Also known as folate hydrolase I (FOLH1) and glutamate carboxypeptidase II (GCPII), PSMA is a type II transmembrane glycoprotein constitutively expressed in healthy human tissue-albeit, at levels up to 1000 times lower than in prostate cancer-such as lacrimal and salivary glands, epididymis, ovary, normal human prostate epithelium, astrocytes and Schwann cells in the nervous central system (CNS) and within the small intestine. PSMA is an enzyme required for the hydrolytic cleavage of γ-linked glutamates from poly-γ-glutamyl folate and the proteolysis of the neuropeptide N-acetyl-L-aspartyl-L-glutamate (NAAG).

Tissue localization and high expression levels relative to non-cancer cells makes PSMA an ideal diagnostic imaging and drug delivery target. However, it has been demonstrated that PSMA propagates signaling upstream of PI3K through G protein-coupled receptors, which contributes to the pathogenesis of prostate cancer. Furthermore, inhibition of PMSA in preclinical models provided a treatment benefit and caused tumor regression.

Embodiments of the present invention comprising specific aptamers that target the PSMA protein while simultaneously delivering siRNAs that inhibit the regulation of key prostate cancer genes will provide novel prostate cancer therapeutics with excellent efficacy to toxicity profiles due to the levels of PSMA overexpression on prostate cancer cells relative to healthy tissue. Kaittanis C et al. Journal of Experimental Medicine 2017.

### PD-1

Programmed cell death protein 1 (PD-1), a member of the immunoglobulin superfamily, is an immune checkpoint protein expressed upon the surface of activated T and B cells. The receptor plays a role in the maintenance of immune tolerance, which is mediated through the binding of its ligands PD-L1 and PD-L2 that are expressed by a variety of cell types, including immune cells such as antigen presenting cells. Tumor cells evade immune surveillance, in part, through their expression of PD-L1. Immune checkpoint inhibitors that block PD-1/PD-L1 interactions augment T cell mediated anti-tumor immunity and have changed the landscape of cancer therapy. Since 2014, the Food and Drug Administration (FDA) has approved multiple anti-PD1 and anti-PD-L1 monoclonal antibodies for the treatment of solid tumors, with pembrolizumab being the first-in-class PD-1 inhibitor that became clinically available. While the majority of patients do not respond to PD-1 blockade, a subset of patients has achieved durable responses. Notably, agents that block immune checkpoints, including PD-1/PD-L1 have been studies in combination clinical studies with other chemotherapy, radiation, cancer vaccines and other checkpoint inhibitors. Based on the results of the Checkmate 227 and 9LA phase 3 studies that enrolled patients with non-small lung cancer (NSCLC), the Food and Drug Administration approved the combination of nivolumab and ipilimumab, monoclonal antibodies that recognize PD-1 and CTLA-4, respectively. Given that tumor-infiltrating lymphocytes (TIL), encounter immunosuppressive conditions in the tumor microenvironment, the efficacy of checkpoint inhibitors may be limited due to an inability to restore sufficient effector function to exhausted T cells that lack the ability to mediate tumor rejection.

Embodiments of the instant invention comprising aptamer-siRNA molecules can reverse T cell exhaustion and enhance T cell-mediated tumor rejection and offers an attractive alternative to the systemic administration of two individual antibody therapies, which can be associated with immune related adverse events. Hellmann et al. (2019) N Engl J Med. 381:2020-31.

### CTLA-4

T cell activation is followed by the upregulation of the immunoglobulin superfamily member, Cytotoxic T lymphocyte antigen-4 (CTLA-4). Engagement of the receptor with its ligands B7.1(CD80) and B7.2(CD86) found on the surface of antigen presenting cells, delivers an inhibitory signal, designed to help maintain immune tolerance and homeostasis. The phenotype of the CTLA-4 knockout mouse confirms that the receptor is a negative regulator of T cell activation, as the animals develop a fatal lymphoproliferative disorder 2-3 weeks after birth. In 2011, ipilimumab became the first checkpoint inhibitor approved by the Food and Drug Administration. The anti-CTLA-4 monoclonal antibody was approved for first- or second-line treatment of patients with advanced melanoma. It has since been approved for the treatment of several additional solid tumors. Ipilimumab, like the PD-1/PD-L1 inhibitors have been evaluated in combination clinical studies designed to leverage drug synergism and limit treatment associated toxicities. Like PD-1, high level of CTLA-4 express on the surface of conventional T cells in the tumor microenvironment is associated with a dysfunctional, or an exhausted state that impedes the ability of T cells to effectively mediate tumor rejection.

Embodiments of the present invention comprise CTLA-4 aptamer-siRNA combinations that minimize T cell exhaustion, increase T cell effector function, and reduce treatment related immune toxicities. Jacob J et al. Adv Pharmacol 2021

### LAG3

The extracellular region of the type 1 transmembrane protein, Lymphocyte (LAG 3/CD223) shares approximately 20% amino acid homology with the T cell expressed protein CD4. However, unlike CD4, the LAG3 intracellular lacks the cysteine motif that would enable the association with lymphocyte-specific protein tyrosine kinase (Lck). Like CD4, MHC Class II is a known ligand of LAG3; however, as LAG3 also regulates the activity of CD8 T cells, it is likely that alternative ligands exist. Two potential LAG3 ligands include, Galectin-3, a known modulator of CD4 and CD8 T cell activity and sinusoidal endothelial cell lectin (LSECtin). LAG3 is an inhibitory receptor that functions to maintain immune tolerance and protect the host from autoimmunity. In the tumor microenvironment (TME), LAG3, along with other inhibitory receptors such as CTLA-4 and PD-1, is upregulated on T cells in response to immunosuppressive cells and soluble factors present in the TME. Tumor infiltrating lymphocytes progressive lose their ability to mediate tumor rejection and acquire and exhausted phenotype. Cancer immunotherapy approaches have been designed to restore T cell function and augment anti-tumor immunity. Presently, there are greater than 30 trials listed in the ClinicalTrials.gov database involving studies with anti-LAG-3 monoclonal antibodies. The majority of which are testing LAG3 antagonistic antibodies in combination with other immune checkpoint inhibitors. Creating aptamer-siRNA therapeutics would enable LAG3 inhibition in combination with other immune checkpoint inhibitors and provide a method for knocking down diverse inhibitory pathways that regulate functions such as inhibitory cytokine signaling and hypoxia, for example. Andrews L P et al. Immunol Rev 2017.

### CD73

The novel immunoinhibitory protein, CD73 (ecto-50-nucleotidase) contributes to tumor growth and metastasis. As an enzyme, CD73 works coordinately with CD39, which converts adenosine triphosphate (ATP) to adenosine diphosphate (ADP). CD73 then breaks down ADP and converts it to adenosine monophosphate (AMP). This process creates an immunosuppressive environment that limits the development of excessive immune responses in normal tissue. By interacting with T cell expressed Adenosine A2A receptors (A2AR) and Adenosine A2B receptors (A2BR) T cells bind immunosuppressive adenosine molecules. Tumors have exploited CD73-mediated adenosinergic mechanism to evade immune responses. In the tumor microenvironment (TME) CD73 and other adenosinergic molecules are inducible on cancer cells and immunosuppressive cell subsets that inhibit intratumoral T cell activity. In addition to the upregulation of CD73 on cancer cells, the protein is expressed on CD8+ T cells in the TME. Therefore, strategies to block the activity of CD73, CD39, or both, have been shown to boost CD8+ T cell responses in a tumor antigen-specific manner. Multiple CD73 antagonists, one small molecule inhibitor, AB680, and several anti-CD73 monoclonal antibodies are currently in early-stage clinical trials.

Embodiments of the instant invention comprising an RNA therapeutic to disrupt signaling through both CD79 and CD39 (on tumor cells or T cells) or inhibiting Adenosine A2AB-A2BR will limit immunosuppression in the TME. Nocentini A et al. Expert Opinion on Therapeutic Patents 2021. Roh M et al. Curr Opin Pharmacol 2020.

### VHL

Under nonmonic conditions, the hypoxia-inducible factors (HIFs), which are heterodimeric transcription factors, are constitutively degraded by a process that requires the von Hippel-Lindau (VHL) complex. The role of VHL as a tumor suppressor is well characterized and loss of VHL function due to spontaneous and inherited mutations causes VHL Syndrome that leads to renal and other specific cancers that may arise in multiple organs; however, HIFs play a role in immunity that may be regulated by VHL. Under conditions of low oxygen (hypoxia) VHL does not interact with the subunits HIF-1α and HIF-2α. As a result, HIF-1α and HIF-2α accumulate and, heterodimerization with HIF-1β and subsequent localization to the nucleus; that results in increased transcription of target genes that allow functional and metabolic adaptations to hypoxic microenvironments. HIF activity has been shown to influence T cell-mediated autoimmunity through the regulation of CD4+ regulatory T cells and TH17 helper T cells. However, the role of HIF-1α and HIF-2α in the differentiation and function of CD8+ T cells in vivo during the response to infection and cancer is poorly understood. Recently, it has been shown that VHL also intrinsically regulates CD8+ T cells. Knockout or knockdown of VHL in CD8+ T cells increases levels of HIF proteins in effector T cells and enables the effector cells to overcome T cell exhaustion, thereby enhancing antitumor immunity.

Embodiments of the instant invention include the administration of aptamer-siRNA molecules that target VHL in conventional T cells to enhance the effector function and boost T cell responses to cancer. Liikanen I et al. Journal of Clinical Investigation 2021

In spite of recent advances, there is a need in the art for compositions and methods of delivering modulators of cell activity (e.g., anti-tumor agents, anti-obesity agents) to cells (e.g., malignant cells, tumor-associated T cells, effector T cells) to inhibit diseases such as cancer, metastasis or metabolic diseases. The nucleic acid compounds and methods of using the same as provided herein solve these and other problems in the art.

### SUMMARY OF THE INVENTION

A multi-targeting siRNA-aptamer platform is provided that is efficiently delivered and is processed by cellular RNAi machinery to produce two siRNAs and at least one siRNA that specifically inhibits expression of two or more different genes. Methods of using the multi-targeting siRNA-aptamer for selectively targeting cells to down-regulate the expression of multiple genes are also provided.

### BRIEF DESCRIPTION OF THE DRAWINGS

FIG. 1: Depicts a bivalent aptamer targeting PD1 and CTLA4 that reverses exhaustion of T cells and helps T cells survive TME.

FIG. 2A: Depicts the potential UBBs1 siRNA targeting sites (highlighted in yellow) on the UBB sequence. Nucleotide differences are highlighted in red (red indicated by solid lines) and similar repeat sequences are in blue (blue indicated by dotted lines). FIG. 2A discloses SEQ ID NO: 326.

FIG. 2B: Depicts the potential UBBs1 siRNA targeting sites (highlighted in yellow) on the UBC sequence. Nucleotide differences are highlighted in red (red indicated by solid lines) and similar repeat sequences are in blue (blue indicated by dotted lines). FIG. 2B discloses SEQ ID NO: 327.

FIG. 3A: Schematic of a potential dual UBB/UBC siRNA aptamer. FIG. 3A discloses SEQ ID NOS 328, 329-330 and 328, respectively, in order of appearance.

FIG. 3B: Schematic of aptamer depicting UBBs1 siRNA and EPCAM aptamer. FIG. 3B discloses SEQ ID NOS 331 and 332, respectively, in order of appearance.

FIG. 4A: Depicts effect of siRNA on HCT-116 colon cancer cell viability.

FIG. 4B: Depicts effect of siRNA on SW480 colon cancer cell viability.

FIG. 5A: Depicts effect of siRNA on HT-29 colon cancer cell viability.

FIG. 5B: Depicts effect of siRNA on RKO colon cancer cell viability.

FIG. 6A: Depicts effect of siRNA on MCF-7 breast cancer cell viability.

FIG. 6B: Depicts effect of siRNA on SK-BR-3 breast cancer cell viability.

FIG. 7A: Dose response curve of UBB targeting siRNA on HCT-116 colon cancer cells.

FIG. 7B: Dose response curve of UBB targeting siRNA on SW480 cancer cells.

FIG. 8A: Depicts effect of U22 siRNA treatment of colon cancer cells on UBB expression normalized to b-Actin.

FIG. 8B: Depicts effect of U22 siRNA treatment of colon cancer cells on UBC expression normalized to b-Actin.

FIG. 8C: Depicts effect of U22 siRNA treatment of colon cancer cells on UBB expression normalized to GAPDH.

FIG. 8D: Depicts effect of U22 siRNA treatment of colon cancer cells on UBC expression normalized to GAPDH.

FIG. 9: Depicts effect of UBB targeting siRNA treatment of HCT-116 colon cancer cells on UBB and UBC expression normalized to GAPDH.

FIG. 10: Depicts effect of UBC targeting siRNA treatment of HCT-116 colon cancer cells on UBB and UBC expression normalized to GAPDH.

FIG. 11A: Depicts alignment of UBB and UBC gene sequences to identify dual targeting siRNA. FIG. 11A discloses SEQ ID NOS 333, 334, 138, 138, 149-153, and 141, respectively, in order of appearance.

FIG. 11B: Depicts alignment of UBB and UBC gene sequences to identify dual targeting siRNA. FIG. 11B discloses SEQ ID NOS 335, 336, 139, 139, 140, 140, 151, 152, 154, and 143, respectively, in order of appearance.

FIG. 11C: Depicts alignment of UBB and UBC gene sequences to identify dual targeting siRNA. FIG. 11C discloses SEQ ID NOS 337, 338, 139, 139, 106, 148, 107, 146, 141, 141, 142, and 142, respectively, in order of appearance.

FIG. 12A: Depicts effect of siRNA on HCT-116 colon cancer cell viability.

FIG. 12B: Depicts effect of siRNA on SK-BR-3 colon cancer cell viability.

FIG. 13A: Depicts alignment of HsUBB and MmUBB to identify dual targeting sequences. FIG. 13A discloses SEQ ID NOS 339, 340, 127, and 343, respectively, in order of appearance.

FIG. 13B: Depicts alignment of HsUBC and MmUBC to identify dual targeting sequences.

FIG. 13B discloses SEQ ID NOS 341, 342, 146, 422, and 344, respectively, in order of appearance.

FIG. 14: Depicts effect of UBB targeting siRNA treatment of HCT-116 colon cancer cells on UBB and UBC expression normalized to GAPDH.

FIGS. 15A-D: Depicts modifications of UBB and UBC targeting siRNA.

FIG. 15A discloses SEQ ID NOS 345, and 346, respectively, in order of appearance.

FIG. 15B discloses SEQ ID NOS 345 and 346, respectively, in order of appearance.

FIG. 15C discloses SEQ ID NOS 347 and 348, respectively, in order of appearance.

FIG. 15D discloses SEQ ID NOS 156 and 348, respectively, in order of appearance.

FIG. 16: Depicts effect of treatment of HCT-116 colon cancer cells with modified UBB targeting siRNA on UBB and UBC expression.

FIG. 17: Depicts effect of treatment of HCT-116 colon cancer cells with modified UBB targeting siRNA on cell viability.

FIG. 18A: Depicts alignment of NR4A1, NR4A2 and NR4A3 gene sequences to identify multitargeting siRNA. FIG. 18A discloses SEQ ID NOS 349-351, 157, 352, 353, 158, 354, 355, 159, 356, and 357, respectively, in order of appearance.

FIG. 18B: Depicts alignment of ADORA2A and ADORA2B gene sequences to identify dual targeting siRNA. FIG. 18B discloses SEQ ID NOS 358-361, 160, 362, 161, 363, 162, and 364, respectively, in order of appearance.

FIG. 18C: Depicts alignment of MAP2K1 and MAP2K2 gene sequences to identify dual targeting siRNA. FIG. 18C discloses SEQ ID NOS 365-368, 163, 369, 164, 370, 165, 371, 166, 166, 167, and 167, respectively, in order of appearance.

FIG. 18D: Depicts alignment of MAPK1 and MAPK3 gene sequences to identify dual targeting siRNA. FIG. 18D discloses SEQ ID NOS 372-375, 168, 376, 169, 377, 170, 378, 171, and 379, respectively, in order of appearance.

FIG. 18E: Depicts alignment of MAPK11 and MAPK14 gene sequences to identify dual targeting siRNA. FIG. 18E discloses SEQ ID NOS 380-383, 172, 172, 173, 173, 174, and 421, respectively, in order of appearance.

FIG. 18F: Depicts alignment of MDM2 and MDM4 gene sequences to identify dual targeting siRNA. FIG. 18F discloses SEQ ID NOS 384, 385, 175, 386, 176, and 387, respectively, in order of appearance.

FIG. 18G: Depicts alignment of PFKFB3 and PFKFB4 gene sequences to identify dual targeting siRNA. FIG. 18G discloses SEQ ID NOS 388-391, 177, 177, 178, and 178, respectively, in order of appearance.

FIG. 19A: Depicts effect of dual targeting siRNA treatment of cancer cells on MAP2K1 and MAP2K2 expression normalized to GAPDH.

FIG. 19B: Depicts effect of dual targeting siRNA treatment of cancer cells on MAPK1 and MAPK3 expression normalized to GAPDH.

FIG. 20A: Depicts effect of dual targeting siRNA treatment of cancer cells on ADORA2A and ADORA2B expression.

FIG. 20B: Depicts effect of dual targeting siRNA treatment of cancer cells on MAPK11 and MAPK14 expression.

FIG. 21A: Depicts effect of gene specific siRNA treatment of cancer cells on NR4A1 expression normalized to GAPDH.

FIG. 21B: Depicts effect of gene specific siRNA treatment of cancer cells on NR4A2 expression normalized to GAPDH.

FIG. 21C: Depicts effect of gene specific siRNA treatment of cancer cells on NR4A3 expression normalized to GAPDH.

FIG. 22: Depicts effect of gene specific siRNA treatment of cancer cells on UBB and UBC expression normalized to GAPDH.

FIG. 23: Depicts effect of gene specific siRNA treatment of cancer cells on AKT1 and BATF expression normalized to GAPDH.

FIG. 24: Schematic depicting dual binding properties of bivalent aptamer-siRNA chimera.

FIG. 25A: Schematic depicting the annealed bivalent EPCAM aptamer-UBB siRNA chimera. FIG. 25A discloses SEQ ID NOS 392 and 393, respectively, in order of appearance.

FIG. 25B: Gel showing comparison of RNA1, RNA2, RNA1 and RNA2 and the annealed EpCAM-directed aptamers-siRNA chimera.

FIG. 26A: Schematic depicting the annealed Her2/Her3 dual targeting aptamer-UBB siRNA chimera. FIG. 26A discloses SEQ ID NOS 394 and 395, respectively, in order of appearance.

FIG. 26B: Gel showing comparison of RNA1, RNA2, and the annealed Her2/Her3 dual targeting aptamer-UBB siRNA chimera.

FIG. 27A: Schematic depicting the annealed EPCAM/Her3 dual targeting aptamer-UBB siRNA chimera. FIG. 27A discloses SEQ ID NOS 396 and 397, respectively, in order of appearance.

FIG. 27B: Schematic depicting the annealed EPCAM/Her3 dual targeting aptamer-Luc siRNA chimera. FIG. 27B discloses SEQ ID NOS 398 and 399, respectively, in order of appearance.

FIG. 27C: Schematic depicting the annealed EPCAM/Her3 dual targeting aptamer-UBB siRNA chimera. FIG. 27C discloses SEQ ID NOS 400 and 401, respectively, in order of appearance.

FIG. 27D: Gel showing comparison of RNA 1, RNA2, and the annealed EPCAM/Her3 dual targeting aptamer-UBB siRNA chimera; RNA3, RNA 4, and the annealed EPCAM/Her3 dual targeting aptamer-Luc siRNA chimera; RNA5, RNA6, and the annealed EPCAM/Her3 dual targeting aptamer-UBB siRNA chimera.

FIG. 27E: Schematic depicting the annealed bivalent EPCAM aptamer-UBB siRNA chimera. FIG. 27E discloses SEQ ID NOS 396 and 400, respectively, in order of appearance.

FIG. 27F: Schematic depicting the annealed bivalent EPCAM aptamer-Luc siRNA chimera. FIG. 27F discloses SEQ ID NOS 398 and 402, respectively, in order of appearance.

FIG. 27G: Gel showing comparison of RNA 7, RNA8, and the annealed bivalent EPCAM aptamer-UBB siRNA chimera; RNA9, RNA10, and the annealed bivalent EPCAM aptamer-Luc siRNA chimera.

FIG. 28A: Schematic depicting the annealed bivalent PSMA aptamer-dual BIRC5 and UBB siRNA chimera. FIG. 28A discloses SEQ ID NOS 403-405, respectively, in order of appearance.

FIG. 28B: Gel showing comparison of RNA 1, RNA2, RNA3 and the annealed bivalent PSMA aptamer-dual BIRC5 and UBB siRNA chimera.

FIG. 29: Depicts the effect of dicer treatment on the PSMA aptamer-dual BIRC5 and UBB siRNA chimera.

FIG. 30A: Schematic depicting annealed EPCAM aptamer-UBB siRNA chimera. FIG. 30A discloses SEQ ID NOS 396 and 347, respectively, in order of appearance.

FIG. 30B: Schematic depicting annealed EPCAM aptamer-Luc siRNA chimera. FIG. 30B discloses SEQ ID NOS 398 and 406, respectively, in order of appearance.

FIG. 30C: Schematic depicting annealed EPCAM aptamer-UBB siRNA chimera. FIG. 30C discloses SEQ ID NOS 396 and 407, respectively, in order of appearance.

FIG. 30D: Depicts the effect of transfection of siRNA or aptamer/siRNA chimeras on UBB expression in cancer cells normalized to GAPDH.

FIG. 31: Depicts the effect of transfection of aptamer/siRNA chimeras on viability of cancer cells normalized to control.

FIG. 32: Depicts the effect of transfection of siRNA on viability of cancer cells normalized to control.

FIG. 33: Depicts the effect of transfection of aptamer/siRNA chimeras on viability of cancer cells normalized to control.

FIGS. 34A-34E Depict predicted folding structures of potential PD1 binding RNA aptamers. FIGS. 34A-34E discloses SEQ ID NOS 230-233, 235, 418, 228, 229, and 225-227, respectively, in order of appearance.

FIGS. 35A-35C: Depict predicted folding structures of potential CTLA4 binding RNA aptamers.

FIGS. 35A-35C discloses SEQ ID NOS 243-247, respectively, in order of appearance.

FIG. 36: Depicts predicted folding structures of potential TROP2 binding RNA aptamers. FIG. 36 discloses SEQ ID NOS 277, 419, 278, and 420, respectively, in order of appearance.

FIGS. 37A-37C: Depict predicted folding structures of potential LAG3 binding RNA aptamers. FIGS. 37A-37C discloses SEQ ID NOS 283-287, respectively, in order of appearance.

FIG. 38A: Provides a table of 5-benzyl Uridine modified RNA aptamer sequences linked to UBB siRNA sequence via nucleotide linker. FIG. 38A discloses SEQ ID NOS 408-412, respectively, in order of appearance.

FIG. 38B: Provides a table of 5-benzyl Uridine modified RNA aptamer sequences linked to UBB siRNA sequence via chemical linker. Figure discloses SEQ ID NOS 348, 413, 348, 414, 348, 415, 348, 416, 348, and 417, respectively, in order of appearance.

FIG. 38C: Schematic of chemical linker for aptamer/siRNA chimera.

FIG. 39: Depicts data from animal studies supporting that NR4A1 is linked to CD8+ T cell dysfunction.

FIG. 40: Depicts transcription factors TOX and NR4A1 as master regulators of exhausted CD8+ T cell

### DETAILED DESCRIPTION OF THE INVENTION

Cancer drugs are most effective when given in combination. One rationale for combination therapy is to use drugs that work by different mechanisms, thereby decreasing the likelihood that resistant cancer cells will develop. When drugs with different effects are combined, each drug can be used at its optimal dose, without intolerable side effects. See for example, https://www.merckmanuals.com/en-ca/home/cancer/prevention-and-treatment-of-cancer/combination-cancer-therapy, accessed May 3, 2021.

Combination therapy may also operate by simultaneously blocking two or more signaling pathways, Wu et al., Nat Biotechnol, 25:1290-1297 (2007). In addition, tumor progression and metastasis may be suppressed by overcoming the functional redundancy or synergistic action of targeted molecules (van der Veeken, et al., Current Cancer Drug Targets, 9:748-760 (2009)).

Zhao, et al. (Cancer discovery. 4.10.1158/2159-8290.CD-13-0465, 2013) discuss the problem of intra-tumor heterogeneity and the approach of using computationally predictive combination therapy to address this problem.

As used herein, the term “oncogene” refers to a gene that can in some circumstances transform a cell into a cancerous cell or a gene that promotes the survival of a cancer cell.

As used herein, the term “effective amount” in the context of the administration of a therapy to a subject refers to the amount of a therapy that achieves a desired prophylactic or therapeutic effect.

A “siRNA,” “small interfering RNA,” “small RNA,” or “RNAi” as provided herein refers to a nucleic acid that forms a double stranded RNA, which double stranded RNA has the ability to reduce or inhibit expression of a gene or target gene (e.g., when expressed in the same cell as the gene or target gene). The complementary portions of the nucleic acid that hybridize to form the double stranded molecule typically have substantial or complete identity. In certain embodiments, a siRNA or RNAi is a nucleic acid that has substantial or complete identity to a target gene and forms a double stranded siRNA. In certain embodiments the “siRNA,” “small interfering RNA,” “small RNA,” or “RNAi” engages the cell's natural RNA-induced silencing complex “RISC” complex to silence genes.

In certain embodiments, the instant invention comprises a chimeric molecule including a cancer marker-binding domain and an inhibitory nucleic acid domain. As used herein, “cancer marker-binding domain” refers to a domain and/or molecule that can bind specifically to a molecule more highly expressed on the surface of a cancer cell as compared to a healthy cell of the same type (a “cancer marker”). As used herein, “inhibitory nucleic acid domain” refers to a domain comprising an inhibitory nucleic acid. In some embodiments, the inhibitory nucleic acid can be a siRNA.

Certain embodiments of the instant invention comprise multi- and multi-multi-targeting siRNA and siRNA-targeting molecule chimeras in treating cancer and other diseases which can be treated by genetic inhibition. The compounds and methods in certain embodiments of the instant invention may utilize one or more aptamers or antibodies that target the therapeutic constructs specifically to cancer cells, providing effective and on-target suppression of the gene or genes targeted by the siRNA.

As used herein “multi-targeting siRNA or construct” refers to a set of unique and novel synthetic molecules for efficacious anti-tumor activity. These constructs each include siRNA molecules that each engage a cell's RNA inhibition system to inhibit more than one different gene (for example UBB and UBC).

As used herein “multi-multi-targeting siRNA or construct” refers to a set of unique and novel synthetic molecules for efficacious anti-tumor activity. These constructs each include siRNA molecules that each engage cell's RNA inhibition system to inhibit more than one different gene and that also include sequences found multiple times within each gene. Such multi-multi-targeting siRNA can be utilized alone or in constructs comprising multiple such siRNAs as well as one or more aptamers. Simple examples of such constructs can be targeted to one or more cancer cells and can inhibit or silence three or four genes although more exotic constructs can readily be envisioned by one skilled in the art once the instant invention is understood.

In certain embodiments of the instant invention each siRNA inhibits two or more different genes.

One embodiment provides a siRNA construct where one siRNA inhibits the expression of UBB and UBC.

One embodiment provides a trivalent siRNA construct where one siRNA inhibits the expression of NR4A1, NR4A2 and optionally NR4A3.

One embodiment provides a siRNA construct where one siRNA inhibits the expression of ADORA2A and ADORA2B.

One embodiment provides a siRNA construct where one siRNA inhibits the expression ADORA2a and ADORA1

One embodiment provides a siRNA construct where one siRNA inhibits the expression of MAP2K1 and MAP2K2.

One embodiment provides a siRNA construct where one siRNA inhibits the expression of ERK1 (MAPK3) and ERK2 (MAPK1).

One embodiment provides a siRNA construct where one siRNA inhibits the expression of MAPK11 and MAPK14.

One embodiment provides a siRNA construct where one siRNA inhibits the expression of MDM2 and MDM4.

One embodiment provides a siRNA construct where one siRNA inhibits the expression of PFKFB3 and PFKFB4.

One embodiment provides a siRNA construct where one siRNA inhibits the expression of ERBB2 and ERBB3.

One embodiment provides a siRNA construct where one siRNA inhibits the expression of OC1 and OC2.

One embodiment provides a siRNA construct where one siRNA inhibits the expression of GRB2 and GRB7.

One embodiment provides a siRNA construct where one siRNA inhibits the expression of IKBKA and IKBKB.

One embodiment provides a siRNA construct where one siRNA inhibits the expression of CDK7 and CDK20.

One embodiment provides a siRNA construct where one siRNA inhibits the expression of ME2 and ME3.

One embodiment provides a siRNA construct where one siRNA inhibits the expression of MSI1 and MSI2.

One embodiment provides a siRNA construct where one siRNA inhibits the expression BCL2 and STAT3.

One embodiment provides a siRNA construct where one siRNA inhibits the expression BCL2 and MYC.

One embodiment provides a siRNA construct where one siRNA inhibits the expression BCL2 and SYK.

One embodiment provides a siRNA construct where one siRNA inhibits the expression BCL2 and Cyclin E2.

One embodiment provides a siRNA construct where one siRNA inhibits the expression E2 and Cyclin D1.

One embodiment provides a siRNA construct where one siRNA inhibits the expression Cyclin D1 and EGFR.

One embodiment provides a siRNA construct where one siRNA inhibits the expression Survivin (BIRC5) Cyclin D2.

One embodiment provides a siRNA construct where one siRNA inhibits the expression of MAP2K1, MEK1, and MAP2K2.

One embodiment provides a siRNA construct where one siRNA inhibits the expression MEK1 and MEK2.

One embodiment provides a siRNA construct where one siRNA inhibits the expression ERK1, MAPK3, and MAPK1.

One embodiment provides a siRNA construct where one siRNA inhibits the expression ERK1 and ERK2.

One embodiment provides a siRNA construct where one siRNA inhibits the expression HIF1 and HIF-2.

One embodiment provides a siRNA construct where one siRNA inhibits the expression TOX and TOX2.

One embodiment provides a siRNA construct where one siRNA inhibits the expression PFKFB2 and PFKFB3.

One embodiment provides a siRNA construct where one siRNA inhibits the expression PLK1 and PLK4.

One embodiment provides a siRNA construct where one siRNA inhibits the expression CDK11A CDK11B.

One embodiment provides a siRNA construct where one siRNA inhibits the expression CDK6 and CDK4.

One embodiment provides a siRNA construct where one siRNA inhibits the expression PARP1 PARP2.

In certain embodiments siRNAs have been experimentally verified by real-time RT-PCR analysis and shown to provide at least 70% target knockdown at the mRNA level when used under optimal delivery conditions (confirmed using validated positive control and measured at the mRNA level 24 to 48 hours after transfection using 100 nM siRNA).

In certain other embodiments, siRNAs have been demonstrated to silence target gene expression by at least 75% at the mRNA level when used under optimal delivery conditions as validated by positive controls and measured at the mRNA level 24 to 48 hours after transfection using 100 nM siRNA.

Another embodiment provides a siRNA-aptamer chimera.

In certain embodiments, an aptamer of the siRNA chimeras binds to a cell surface protein expressed on cancer cells.

In certain embodiments, an aptamer of the siRNA chimeras specifically bind to epithelial cell adhesion molecules (EpCAM), a glycosylated membrane protein.

In certain embodiments, an aptamer of the siRNA chimeras specifically bind to DExH-Box Helicase 9, DHXP ((NCBI Gene ID: 1660). DHX9 protein is involved in transcriptional and translational regulation, DNA replication/repair, and maintenance of genome stability. DHX9 has been shown to shuttle between the nucleus and the cytoplasm.

In certain embodiments, a method and constructs are provided that include administering to a subject in need thereof and effective amount of an RNA construct having at least one aptamer that specifically binds to EPCAM and siRNA that are processed to produce siRNA that inhibits expression of UBB and UBC; NR4A1, NR4A2 and NR4A3; ADORA2A and ADORA2B; MAP2K1 and MAP2K2; ERK1 (MAPK3) and ERK2 (MAPK1); MAPK11 and MAPK14; MDM2 and MDM4; PFKFB3 and PFKFB4; TOX and TOX2.

In another embodiment RNA construct having one aptamer that specifically binds to EPCAM and another aptamer that specifically binds to HER3 and siRNA that are processed to produce siRNA that inhibits expression of UBB and UBC.

Another embodiment provides a pharmaceutical composition containing one or more different bivalent siRNA chimeras in an amount effective to down down-regulate at least three different genes in a target cell.

The method includes administering a dual targeting siRNA agent to the subject to be treated. When the organism to be treated is a mammal such as a human, the composition can be administered by any means known in the art including, but not limited to oral or parenteral routes, including intravenous, intramuscular, subcutaneous, transdermal, and airway (aerosol) administration. In some embodiments, the compositions are administered by intravenous infusion or injection.

Ubiquitin B (UBB) is one of the two genes that encode for Ubiquitin. Silencing of UBB results in dependence on the second gene, Ubiquitin C (UBC) (Tsherniak et al., Cell, 170: 564-576(2017)). In certain embodiments described herein UBB and UBC can be effectively targeted with a single siRNA.

UBB and UBC also contain conserved regions that could be exploited as a means to target both genes in multiple locations with one siRNA. Targeting multiple genes in multiple locations will be defined as multi-multi-targeting. Thus, a UBB/UBC siRNA can be designed as a multi-multi-targeting siRNA construct. When included in an siRNA/aptamer chimera including more than one aptamer or other targeting entity, the construct actually can be thought of as a multi-multi-multi-targeting molecule.

In certain embodiments, a preferred aptamer for conjugation to a multi-targeting siRNA is an epithelial cell adhesion molecule (EpCAM) aptamer, EpCAM is a glycosylated membrane protein that is expressed in most organs and glands, with the highest expression in colon. (Schnell et al., BBA—Biomembranes, 1828: 1989-2001(2013)). Sequences for EpCAM are known for a variety of species, e.g., human EpCAM (see, e.g., NCBI Gene ID:4072; protein sequence: NCBI Ref Seq: NP_002345.2). in one embodiment, a single EpCAM aptamer consisting of 19-nt RNA possesses similar binding affinity as antibodies and is efficiently internalized through receptor-mediated endocytosis (Shigdar, et al., Cancer Sci, 102:991-998 (2011); Wang, et al., Theranostics, 5:1456-1472 (2015)). Additionally, EpCAM is highly expressed in colon cancers and associated with colon cancer cell migration, proliferation, metastasis, and poor prognosis (Liang et al., Cancer Letters, 433: 165-175(2018)). For these reasons EpCAM has been used in certain embodiments of the instant invention as an aptamer target for targeted delivery of therapeutic siRNAs for colon cancer.

In certain embodiments, the aptamers described herein, for example those targeting EpCAM, permit the therapy to target tumor-initiating cells (also referred to as cancer stem cells). These cells are responsible not only for tumor initiation, relapse, and metastasis, but are also relatively resistant to conventional cytotoxic therapy. Thus, the compositions and methods described herein permit effective treatment of the underlying pathology in a novel way that existing therapies fail to do.

Moreover, the compounds according to certain embodiments of the instant invention are expected to be surprisingly efficacious in the treatment of colon cancers.

The compounds according to certain embodiments of the instant invention are effective to inhibit gene expression in tumor cells.

In certain embodiments, the instant invention is also designed for targeted delivery of the therapeutic constructs and thus rapid tumor treatment.

In certain embodiments, the cancer marker can be a protein and/or polypeptide. In certain embodiments, one cancer marker can be EpCAM. In certain preferred embodiments, the cancer marker-binding domain can be an aptamer.

Additional cancer markers that are targeted by the aptamer portion of certain embodiments of the instant invention include, but are not limited to, ERBB2, ERBB3, PSMA, FOLH1, CD44, FOLH1, PSCA, PDCD1, TACSTD2, NT5E, PDCD1, CTLA4, LAG3, DHX9, CD73, PD-1, Tim-3 or HAVCR2.

In certain embodiments, an aptamer-siRNA chimera according to the instant invention includes an aptamer targeting ERBB2 (HER2) (NCBI Gene ID: 2064). HER2, a membrane tyrosine kinase, is overexpressed in 20%-30% of breast cancer and correlates with poor prognosis, high aggressiveness, and extensive drug resistance. U.S. Pat. No. 10,960,086 discloses an aptamer targeting HER2 as part of an siRNA-aptamer chimera.

In certain embodiments, an aptamer-siRNA chimera according to the instant invention includes an aptamer targeting ERBB3 (HER3) (NCBI Gene ID: 2065). HER3, a membrane tyrosine kinase, is involved in the resistance against EGFR- and HER2-targeted therapies through activation of a compensatory survival pathway. U.S. Pat. No. 10,960,086 (incorporated herein by reference) discloses an aptamer targeting HER3 as part of an siRNA-aptamer chimera.

In certain embodiments, an aptamer-siRNA chimera according to the instant invention includes an aptamer targeting PSMA (NCBI Gene ID: 2346). Prostate-specific membrane antigen is a transmembrane protein expressed in all types of prostatic tissue. PSMA expression correlates

In certain embodiments, an aptamer-siRNA chimera according to the instant invention includes an aptamer targeting CD44 (NCBI Gene ID: 960). CD44 is a transmembrane glycoprotein whose aberrant expression and dysregulation contributes to tumor initiation and progression. CD44 is involved in many processes including T cell differentiation, branching morphogenesis, proliferation, adhesion and migration. CD44 is a common biomarker of cancer stem cells.

In certain embodiments, an aptamer-siRNA chimera according to the instant invention includes an aptamer targeting EPCAM (NCBI Gene ID: 4072). EPCAM is a glycosylated membrane protein that is expressed in most organs and glands, with the highest expression in colon and is associated with colon cancer cell migration, proliferation, metastasis, and poor prognosis. A single EpCAM aptamer consisting of 19-nt RNA possesses similar binding affinity as antibodies and is efficiently internalized through receptor-mediated endocytosis (Shigdar, et al., Cancer Sci, 102:991-998 (2011).

In certain embodiments, an aptamer-siRNA chimera according to the instant invention includes an aptamer targeting PSCA, prostate stem cell antigen (NCBI Gene ID: 8000). PSCA is a membrane glycoprotein predominantly expressed in the prostate with a possible role in cell adhesion, proliferation control and cell survival. PSCA can have a tumor promoting or a tumor suppressive effect depending on the cell type.

In certain embodiments, an aptamer-siRNA chimera according to the instant invention includes an aptamer targeting PD1 (NCBI Gene ID: 5133) PD1 is an immune checkpoint molecule exploited by tumors to dampen T cell activation and avoid autoimmunity and the effects of an inflammatory response. Inhibiting PD1 enhances anti-tumor immunity.

In certain embodiments, an aptamer-siRNA chimera according to the instant invention includes an aptamer targeting CTLA4 (NCBI Gene ID: 1493). CTLA4 is an immune checkpoint molecule whose expression is dysregulated in tumors and in tumor-associated T cells. (Santulli-Marotto, S. et al., Cancer Res 63:7483-7489 (2003)). U.S. patent application Ser. No. 16/892,995 provides a CTLA-4 aptamer conjugated to an antisense nucleic acid.

In certain embodiments, an aptamer-siRNA chimera according to the instant invention includes an aptamer targeting TROP2 (NCBI Gene ID: 4070). TROP2, a cell-surface glycoprotein, is a paralog of epithelial-specific cell adhesion molecule (EpCAM). It is overexpressed in adenocarcinomas, minimally expressed in normal tissues, and expression level is correlated with tumor invasiveness and poor prognosis.

In certain embodiments, an aptamer-siRNA chimera according to the instant invention includes an aptamer targeting CD73 (NCBI Gene ID: 4907). CD73 is part of an enzyme cascade to breakdown ATP into adenosine. Overexpression of CD73 occurs in many cancers and leads to overproduction of adenosine which suppresses the antitumor immune response and helps aid cancer proliferation, angiogenesis, and metastasis.

In certain embodiments, an aptamer-siRNA chimera according to the instant invention includes an aptamer targeting LAG-3 (NCBI Gene ID: 3902). LAG3, cell surface molecule, is primarily expressed on activated T cells and NK cells and is a marker for the activation of CD4+ and CD8+ T cells. The co-expression of LAG3 with other inhibitory molecules including PD-1 induces T cell exhaustion.

In certain embodiments, an aptamer-siRNA chimera according to the instant invention includes an aptamer targeting TIM-3 (NCBI Gene ID: 84868). TIM-3, cell surface molecule, is constitutively expressed on innate immune cells and suppresses innate antitumor immunity by mediating T-cell exhaustion. TIM-3 is co-expressed with PD-1 and is upregulated during PD-1 blocking therapy. Blocking the TIM-3 pathway enhances cancer immunity and increases interferon-gamma (IFN-v) in T cells.

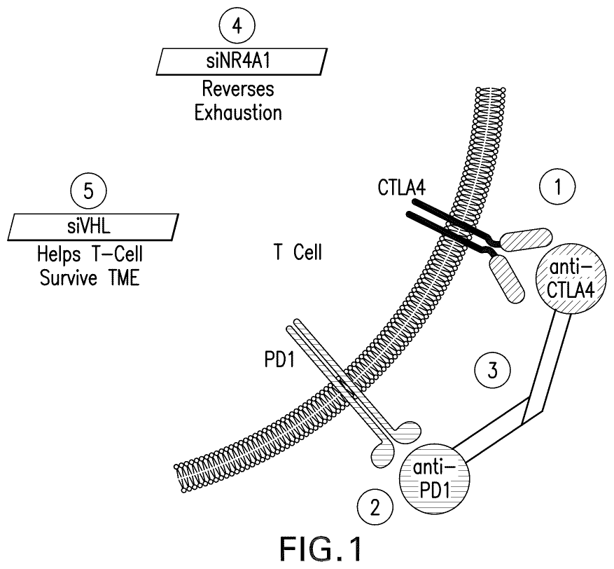

This first-in-class, bivalent aptamer-dual siRNA chimera harnesses the immune stimulatory potential of CTLA-4 and PD-1 within one RNA molecule. The results of the Phase III Checkmate 227 clinical trial in advanced non-small cell lung cancer recently demonstrated the longer duration of overall survival compared with chemotherapy in patients with NSCLC (Hellmann et al., N Engl J Med, 2019). In addition to delivering CTLA-4 and PD-1 antagonists selectively to T cells, this bivalent aptamer carries siRNA silencers that knock down expression of NR4A1, which reinvigorates exhausted T cells and VHL, which enables cells to adapt to hypoxic conditions in the TME. (FIG. 1)

The inhibitory nucleic acid domain of constructs according to certain embodiments of the instant invention can inhibit the expression of a gene product that is upregulated in a cancer cell and/or the expression of a gene that is required for cell growth and/or survival. In some embodiments, the inhibitory nucleic acid domain can inhibit the expression of a gene selected from UBB (e.g., “Ubiquitin B”; NCBI Gene ID: 7314); UBC (e.g. “Ubiquitin C”; NCBI Gene ID: 7316), BCL2, STAT3, MYC, SYK, CCNE2, CCND1, CCND2, BIRC5, EGFR, UBB, UBC, NR4A1, NR4A2, NR4A3, ADORA2a, ADORA2b, ADORA1, MAP2K1, MAP2K2, MAPK3 (ERK1), MAPK1 (ERK2), HIF1, HIF2, PFKFB3, PFKFB4, PLK1, PLK4, CDK11A, CDK11B, CDK4, CDK6, PARP1, or PARP2. Sequences of these genes, e.g., the human mRNAs, may be obtained from the NCBI database and can be used according to the instant invention to inhibitory nucleic acids.

Furthermore, provided herein are exemplary inhibitory nucleic acids, e.g., a nucleic acid having the sequence of SEQ ID NO: 107.

Ubiquitin B (UBB) is one of the two genes that encode for Ubiquitin. Silencing of UBB results in dependence on the second gene, Ubiquitin C (UBC) (Tsherniak et al., Cell, 170: 564-576(2017)). Targeting of UBC in high-grade serous ovarian cancer (HGSOC), a cancer known for chronic UBB repression, demonstrated tumor regression and long term survival benefits.

According to the instant invention the relationship between UBB and UBC provides a dual targeting pathway by targeting both UBB and UBC as a therapeutic strategy for cancer (Kedves, et al., Clin Invest, 127: 4554-4568 (2017)).

In certain embodiments, a siRNA according to the invention targets BCL2 (NCBI Gene ID:596) which is a regulator of apoptosis that is triggered in response to stress signals. BCL-2 was the first gene shown to promote prolonged cell survival rather than increased proliferation leading to the concept that inhibition of apoptosis is an important step in tumorigenesis.

In certain embodiments, a dual-targeting siRNA targets BCL2 and STAT3 (NCBI Gene ID: 6774) which is a cytoplasmic transcription factor that regulates cell proliferation, differentiation, survival, angiogenesis, and immune response.

In certain embodiments, a dual-targeting siRNA targets BCL2 and MYC (NCBI Gene ID: 4609) which is a proto-oncogene and encodes a nuclear phosphoprotein that plays a role in cell cycle progression, apoptosis and cellular transformation. Reregulated expression of MYC causally contributes to tumorigenesis and tumor growth maintenance.

In certain embodiments, a dual-targeting siRNA targets BCL2 and SYK (NCBI Gene ID: 6850), Spleen Associated Tyrosine Kinase, which has a cancer dependent therapeutic function. In many hematopoietic malignancies, SYK provides a survival function and inhibition or silencing of SYK can promote apoptosis. In cancers of non-immune cells, SYK can suppress tumorigenesis by enhancing cell-cell interactions and inhibiting migration.

In certain embodiments, a dual-targeting siRNA targets BCL2 and Cyclin E2 (NCBI Gene ID: 9134), a member of the cyclin family that assists in regulating the cell cycle and whose expression has been associated with chemotherapy resistance of tumor cells and poor prognosis.

In certain embodiments, a dual-targeting siRNA targets Cyclin E2 and Cyclin D1 (NCBI Gene ID: 595). Cyclin D1 overexpression is predominantly correlated with early cancer onset, tumor progression, shorter cancer patient survival and increased metastases.

In certain embodiments, a dual-targeting siRNA targets Cyclin D1 and EGFR (NCBI Gene ID: 1956), epidermal growth factor receptor, a cell surface protein whose expression modulates growth, signaling, differentiation, adhesion, migration and survival of cancer cells.

In certain embodiments, a dual-targeting siRNA targets Survivin (BIRC5) (NCBI Gene ID: 332) and Cyclin D2 (NCBI Gene ID: 895). Expression of Survivin in tumors correlates with inhibition of apoptosis, resistance to chemotherapy, and tumor progression. Cyclin D2 overexpression has a critical role in cell cycle progression and the tumorigenicity and suppression of cyclin D2 expression has been linked to G1 arrest in vitro.

In certain embodiments, a dual-targeting siRNA targets NR4A1 (NCBI Gene ID: 3164) and NR4A2 (NCBI Gene ID: 4929). When T cells encounter sustained T cell stimulation through exposure to self-antigens, to chronic infections or to the tumor microenvironment, then effector T cells may become dysfunctional to avoid excessive immune responses, which is known as T-cell exhaustion. NR4A1, a driver of cancer cell survival, has been identified as a key mediator of T cell dysfunction and contributor of regulatory T-cell-mediated suppression of anti-tumor immunity in the tumor microenvironment. Nr4a2 is highly expressed in tumor-infiltrating cells than in bystander cells. Furthermore, mice lacking Nr4a1 and Nr4a2 genes specifically in Tregs showed resistance to tumor growth in transplantation models.

In certain embodiments, a dual-targeting siRNA targets NR4A1 and NR4A3 (NCBI Gene ID: 8013), which is expressed similarly to NR4A1.

In certain embodiments, a multi-targeting siRNA targets NR4A1, NR4A2, and NR4A3.

In certain embodiments, a dual-targeting siRNA targets ADORA2a (NCBI Gene ID: 135) and ADORA2b (NCBI Gene ID: 136). ADORA2a signaling during T cell activation strongly inhibited development of cytotoxicity and cytokine-producing activity in T cells, whereas the inhibition of T cell proliferation was only marginal. While an adenosine-rich environment may allow for the expansion of T cell, it impairs the functional activation of T cells. Targeting the ADORA2a immunosuppressive pathway restores both effector function and metabolic fitness of peripheral and tumor-derived CD8⁺ T cells. ADORA2b promotes the expansion of myeloid-deriver suppressor cells which are immunosuppressive cells that promote tumor progression by impairing antitumor T-cell responses and/or modulating angiogenesis. Experiments targeting both ADORA2a and ADORA2b have shown greater infiltration by CD8⁺ T cells as well as NK cells, and they encompass fewer Tregs.

In certain embodiments, a dual-targeting siRNA targets ADORA2a and ADORA1 (NCBI Gene ID: 134). ADORA1 and ADORA2A are paralogues and high-affinity receptors responding to low concentrations of extracellular adenosine.

In certain embodiments, a dual-targeting siRNA targets MAP2K1 (NCBI Gene ID: 5604), MEK1, and MAP2K2 (NCBI Gene ID: 5605), MEK2. MEK1 and MEK2 are closely related and participate in the Ras/Raf/MEK/ERK signal transduction cascade. MEK1 and MEK2 are the exclusively specific activators of ERK1/2, and their inhibition could result in the clinical benefits for treatment of cancers with RAS/RAF dysfunction.

In certain embodiments, a dual-targeting siRNA targets MAPK3 (NCBI Gene ID: 5595), ERK1, and MAPK1 (NCBI Gene ID: 5594) ERK2. ERK1 and ERK2, which are homologous by 85%, are part of the MAPK pathway, and the only substrate or MEK. The Ras-dependent extracellular signal-regulated kinase (ERK)1/2 mitogen-activated protein (MAP) kinase pathway plays a central role in cell proliferation control. ERK1/2 inhibitors can reverse the abnormal activation of MAPK pathway induced by upstream mutations including RAS mutation (Liu et al).

In certain embodiments, a dual-targeting siRNA target HIF1 (NCBI Gene ID: 3091) and HIF-2 (NCBI Gene ID: 2034). Hypoxia inducible factor (HIF)-1 and HIF-2 are heterodimeric transcription factors mediating the cellular response to hypoxia.

In certain embodiments, a dual-targeting siRNA target TOX (NCBI Gene ID: 9760) and TOX2 (NCBI Gene ID: 84968). High-mobility group (HMG)-box transcription factors, TOX and TOX2, are critical for the transcriptional program of CD8⁺ T cell exhaustion downstream of NFAT.

In certain embodiments, a dual-targeting siRNA targets PFKFB2 (NCBI Gene ID: 5208) and PFKFB3 (NCBI Gene ID: 5209). PFKFB2 is overexpressed in pancreatic adenocarcinomas and functions to regulate glycolysis and proliferation in pancreatic cancer cells. PFKFB3 is important for maintaining metabolic functions in pancreatic cancers and may be involved in providing a localized ATP supply at the plasma membrane.

In certain embodiments, a dual-targeting siRNA targets PFKFB3 and PFKFB4 (NCBI Gene ID: 5210). PFKFB4 is regulatory enzyme synthesizes a potent stimulator of glycolysis and is over expressed in many types of cancer such as in glioma, lung, and prostate cancers.

In certain embodiments, a dual-targeting siRNA targets PLK1 (NCBI Gene ID: 5347) and PLK4 (NCBI Gene ID: 10733). Polo-like kinase 1 and 4 play an important role in the initiation, maintenance, and completion of mitosis. Dysfunction of PLK1/4 promotes tumorigenesis. PLK1/4's role in cellular growth and proliferation and overexpression in multiple types of human cancer and has made them an attractive dual target.

In certain embodiments, a dual-targeting siRNA targets CDK11A (NCBI Gene ID: 728642) and CDK11B (NCBI Gene ID: 984). Recent studies have found that the overexpression and activation of CDK11 is crucial in the growth and proliferation of cancer cells, including breast cancer, multiple myeloma, osteosarcoma, and other types of cancer. Both of genes contain 20 exons and 19 introns that encode almost identical protein kinases, CDK11A and CDK11B.

In certain embodiments, a dual-targeting siRNA targets CDK6 (NCBI Gene ID: 1021) and CDK4 (NCBI Gene ID: 1432). CDK4/6 is highly expressed in the majority of human cancers through a multitude of genomic alterations. Sustained activation of CDK4/6 encourages cancer cells to enter the cell cycle continuously by shortening the duration of the G1 phase. CDK4/6 is highly expressed in the majority of human cancers through a multitude of genomic alterations. Sustained activation of CDK4/6 encourages cancer cells to enter the cell cycle continuously by shortening the duration of the G1 phase.

In certain embodiments, a dual-targeting siRNA targets MAPK11 (NCBI Gene ID: 5600) and MAPK14 (NCBI Gene ID: 1019). Mitogen activated protein kinases are involved in signaling transduction pathways, cell survival, differentiation, proliferation and apoptosis. MAPK11 has been found to be hypermethylated with a slight increase of expression in Breast, Uterine Endometrial, Cervical, Ovarian and Uterine Carcinosarcoma cell samples. MAPK11's functions are mostly redundant to MAPK14 making these genes a strong dual target.

In certain embodiments, a dual-targeting siRNA targets MDM2 (NCBI Gene ID: 4193) and MDM4 (NCBI Gene ID: 4194). MDM2 and MDM4 are inhibitors of p53 expression. Dual inhibition of these genes has been shown to inhibit cellular proliferation by inducing cell cycle arrest and apoptosis in certain cancers.

In certain embodiments, a dual-targeting siRNA targets PARP1 (NCBI Gene ID: 142) and PARP2 (NCBI Gene ID: 10038). PARP is an important player in the DNA repair pathway which decreases cytotoxicity of chemotherapies and other. Targeted inhibition of PARP in cancerous cells assists in promoting cytotoxicity especially in combination with another therapy.

TABLE 1
siRNAs useful in certain embodiments of the instant invention. Each of the siRNAs
disclosed target two different genes. The sense strands, when paired with their
complementary guide strands as part of the constructs according to certain embodiments of
the invention are processed by cellular machinery to produce siRNAs wherein the siRNAs
specifically inhibit expression of two or more different genes.

| Gene Targets | siRNA (Sense Strand) | SEQ ID NO |
|---|---|---|
| ERBB2ERBB3 | CACCAUUGAUGUCUACAUG | 1 |
| OC10C2 | CAGCAACUUCUUCAUGAAC | 2 |
| OC20C3 | CAUCUUCAAGGAGAACAAA | 3 |
| GRB2GRB7 | UGCCAGGAGGGGGCAGCUC | 4 |
| IKBKAIKBKA | UGCAGUAUCUGGCCCCAGA | 5 |
| CDK7CDK20 | CUACAUAGGGAUCUGAAAC | 6 |
| ME2ME3 | UUUGCCAGUGGCAGUCCAU | 7 |
| MSI1MSI2 | GAAUGUAAGAAAGCUCAGC | 8 |
| ADORA2AADORA1 | GUCUACUUCAACUUCUUUG | 9 |
| ADORA2AADORA2B | CCUCACGCAGAGCUCCAUC | 10 |
| ADORA2AADORA2B | CCUCACCCAGAGCUCCAUC | 11 |
| ADORA2AADORA2B | CAUGGUGUACUUCAACUUC | 12 |
| ADORA2AADORA2B | CAUGGUCUACUUCAACUUC | 13 |
| ADORA2AADORA2B | GUGUACUUCAACUUCUUUG | 14 |
| ADORA2AADORA2B | GUCUACUUCAACUUCUUUG | 15 |
| MAP2K1 (MEK1)MAP2K2 (MEK2) | AAUCCGGAACCAGAUCAUA | 16 |
| MAP2K1 (MEK1)MAP2K2 (MEK2) | CAUCCGGAACCAGAUCAUC | 17 |
| MAP2K1 (MEK1)MAP2K2 (MEK2) | GUACAUCGUGGGCUUCUAU | 18 |
| MAP2K1 (MEK1)MAP2K2 (MEK2) | GUACAUCGUGGGCUUCUAC | 19 |
| MAP2K1 (MEK1)MAP2K2 (MEK2) | CAAGCCCUCCAACAUCCUA | 20 |
| MAP2K1 (MEK1)MAP2K2 (MEK2) | GAAGCCCUCCAACAUCCUC | 21 |
| MAP2K1 (MEK1)MAP2K2 (MEK2) | GAUCAAGCUCUGUGACUUU | 22 |
| MAP2K1 (MEK1)MAP2K2 (MEK2) | GAUCAAGCUGUGUGACUUC | 23 |
| MAP2K1 (MEK1)MAP2K2 (MEK2) | UCGACUCCAUGGCCAACUC | 24 |
| MAP2K1 (MEK1)MAP2K2 (MEK2) | CAUGGCCAACUCCUUCGUG | 25 |
| MAPK3 (ERK1)MAPK1 (ERK2) | UGAGCAAUGACCAUAUCUG | 26 |
| MAPK3 (ERK1)MAPK1 (ERK2) | UCAGCAAUGACCAUAUCUG | 27 |
| MAPK3 (ERK1)MAPK1 (ERK2) | CUACUUCCUCUACCAGAUC | 28 |
| MAPK3 (ERK1)MAPK1 (ERK2) | CUAUUUUCUCUACCAGAUC | 29 |
| MAPK3 (ERK1)MAPK1 (ERK2) | CCAAGGGCUAUACCAAGUC | 30 |
| MAPK3 (ERK1)MAPK1 (ERK2) | CCAAGGGCUACACCAAGUC | 31 |
| MAPK3 (ERK1)MAPK1 (ERK2) | GUCUGUGGGCUGCAUUCUG | 32 |
| MAPK3 (ERK1)MAPK1 (ERK2) | GUCUGUAGGCUGCAUUCUG | 33 |
| MAPK3 (ERK1)MAPK1 (ERK2) | CUCUAACCGGCCCAUCUUC | 34 |
| MAPK3 (ERK1)MAPK1 (ERK2) | CUCUAACAGGCCCAUCUUC | 35 |
| MAPK3 (ERK1)MAPK1 (ERK2) | GGAGGACCUGAAUUGUAUC | 36 |
| MAPK3 (ERK1)MAPK1 (ERK2) | AGAAGACCUGAAUUGUAUA | 37 |
| HIF1HIF2 | GAAAGCCUUGGAUGGUUUU | 38 |
| TOXTOX2 | UACUCAGGCCGCCAUCAAG | 39 |
| PFKFB3PFKFB4 | AGACCUACAUCUCCAAGAA | 40 |
| PLK1PLK4 | GUGGGACUCCUAAUUACAU | 41 |
| CDK11ACDK11B | CAAGAAAACAGAUGAAAUU | 42 |
| CDK4CDK6 | GUUUCGUCGAAAGCCUCUC | 43 |
| PARP1PARP2 | CCAAGAGUGCCAACUACUG | 44 |
| NR4A1NR4A2 | AGCGCACAGUGCAGAAAAA | 45 |
| NR4A1NR4A2 | UGCUGUGUGUGGGGACAAC | 46 |
| NR4A1NR4A2 | CGCUGUGUGUGGGGACAAC | 47 |
| NR4A1NR4A2 | AUGUGAGGGCUGCUGCAAG | 48 |
| NR4A1NR4A2 | CUGUGAGGGCUGCUGCAAA | 49 |
| NR4A1NR4A2 | CUGCAAGGGCUUCUUCAAG | 50 |
| NR4A1NR4A2 | CUGCAAAGGCUUCUUUAAG | 51 |
| NR4A1NR4A2 | GGGCUGCAAGGGCUUCUUC | 52 |
| NR4A1NR4A2 | GGGCUGCAAAGGCUUCUUU | 53 |
| NR4A1NR4A2 | GCGCACAGUGCAGAAAAAC | 54 |
| NR4A1NR4A2 | GCGCACAGUGCAAAAAAAC | 55 |
| NR4A1, NR4A2, NR4A31 | UGCUGUGUGUGGGGACAAC | 56 |
| NR4A1, NR4A2, NR4A32 | CGCUGUGUGUGGGGACAAC | 57 |
| NR4A1, NR4A2, NR4A33 | UGCCGUGUGCGGGGACAAC | 58 |
| NR4A1, NR4A2, NR4A31 | CUGCAAGGGCUUCUUCAAG | 59 |
| NR4A1, NR4A2, NR4A32 | CUGCAAAGGCUUCUUUAAG | 60 |
| NR4A1, NR4A2, NR4A33 | CUGCAAGGGCUUUUUCAAG | 61 |
| NR4A1, NR4A2, NR4A31 | GGGCUGCAAGGGCUUCUUC | 62 |
| NR4A1, NR4A2, NR4A32 | GGGCUGCAAAGGCUUCUUU | 63 |
| NR4A1, NR4A2, NR4A33 | GGGCUGCAAGGGCUUUUUC | 64 |
| NR4A1, NR4A2, NR4A31 | GCGCACAGUGCAGAAAAAC | 65 |
| NR4A1, NR4A2, NR4A32 | GCGCACAGUGCAAAAAAAC | 66 |
| NR4A1, NR4A2, NR4A33 | GAGAACAGUGCAGAAAAAU | 67 |
| NR4A1, NR4A2, NR4A31 | ACUGCCCUGUGGACAAGAG | 68 |
| NR4A1, NR4A2, NR4A32 | ACUGCCCAGUGGACAAGCG | 69 |
| NR4A1, NR4A2, NR4A33 | ACUGCCCAGUAGACAAGAG | 70 |
| ME1ME2 | UUCAAUGAUGAUAUUCAAG | 71 |
| KRASNRAS | AUCCAACAAUAGAGGAUUC | 72 |
| KRASHRAS | UCAUUUUGUGGACGAAUAU | 73 |
| BRAFARAF | GUGGUGUGAGGGCUCCAGC | 74 |
| BRAFRAF1 | CUUACAACACACAACUUUG | 75 |

TABLE 2
siRNAs useful in certain embodiments of the
instant invention. As disclosed in Tiemann et al,
RNA (2010), 16:1275-1284 (incorporated herein
by reference). In each siRNA the sense strand
inhibits a first gene and the guide strand
inhibits a second gene. This approach is limited
by the rarity of useful sequences of this type
but these siRNA are useful in certain
embodiments of the invention where it is
desired to target the two listed genes.

| Gene Targets | SIRNA (Sense Strand) | SEQ ID NO |
|---|---|---|
| BCL2STAT3 | AAUUGAGGUGCUUCCUUAAUUGUga | 76 |
| BCL2STAT3 | UCACAAUCAGGGAAGCAUCACAAUUGG | 77 |
| BCL2STAT3 | AAUUGAGGUGCUUCCUUAAUU | 78 |
| BCL2STAT3 | UCAGGGAAGCAUCACAAUUGG | 79 |
| BCL2MYC | AUUUGAGGCAGUUUACAUUUCGGtg | 80 |
| BCL2MYC | CACCGAAAUGUUCACUUCCUCAAGUUC | 81 |
| BCL2MYC | AUUUGAGGCAGUUUACAUUUC | 82 |
| BCL2MYC | AAUGUUCACUUCCUCAAGUUC | 83 |
| BCL2SYK | UUUCCAUUUGUCUUUGAUCUUUGtt | 84 |
| BCL2SYK | AACAAAGAUCACAUAUAAAUGGAAGGC | 85 |
| BCL2SYK | UUUCCAUUUGUCUUUGAUCUU | 86 |
| BCL2SYK | GAUCACAUAUAAAUGGAAGGC | 87 |
| BCL2CCNE2 | UUCCUCAAGUUUGGAAGCAGCCUtg | 88 |
| BCL2CCNE2 | CAAGGCUGAUUCUAAACUGGAAGAAAA | 89 |
| CCNE2CCND1 | ACCAUCAGUGACGUAAGCAAAAGaa | 90 |
| CCNE2CCND1 | UUCUUUUGCUUAAGUCAGAGAUGGAAG | 91 |
| CCND1EGFR | AUUCCAAUGCCAUCCACUUUCAGtc | 92 |
| CCND1EGFR | GACUGAAAGUGCUUGGAAAUGGAAUGG | 93 |
| BIRC5CCND2 | UCCAUUUACAGACUGACACUUUCca | 94 |
| BIRC5CCND2 | UGGAAAGUUACAGACUGUAAAUAGAGU | 95 |

Certain embodiments of the instant invention include a linker as outline in U.S. Pat. No. 10,960,086 incorporated herein by reference in its entirety. Alternative linkers can be substituted. 2-4 unpaired bases have been demonstrated to be sufficient to retain aptamer function. However, U's can be substituted in place of the A's. Additionally, in certain embodiments a streptavidin disulfide linker can be used (Ted et al., Nucleic Acid Research, 2006). The aptamers and siRNAs can be tethered to complementary linker sequences and hybridized together through Watson-Crick base pairing (Pastor et al., Mol Ther, 2011). Additionally, siRNA and aptamers can be tethered through a 4 nt (CUCU) linker or covalently fused through 2 nt linker (UU) (Zhou et al, Mol Ther, 2008) (Zhou et el., Theranostics, 2018). The aptamers and siRNAs can also be bound through a “sticky bridge” of 16 nt repeating GC with a three carbon spacer on either side of the sticky bridge (Zhou et al., Nucleic Acids, 2009). The aptamers and siRNAs can be conjugated with an acid-labile linkage or a kissing loop interaction (Huang et al., Chembiochem. 2009) (Guo et al., Human Gene Therapy, 2005).

In some embodiments, the methods described herein relate to treating a subject having or diagnosed as having cancer with a composition as described herein. Subjects having cancer can be identified by a physician using current methods of diagnosing cancer.

In some embodiments, the pharmaceutical composition as described herein can be a parenteral dose form. Since administration of parenteral dosage forms typically bypasses the patient's natural defenses against contaminants, parenteral dosage forms are preferably sterile or capable of being sterilized prior to administration to a patient. Examples of parenteral dosage forms include, but are not limited to, solutions ready for injection, dry products ready to be dissolved or suspended in a pharmaceutically acceptable vehicle for injection, suspensions ready for injection, and emulsions. In addition, controlled-release parenteral dosage forms can be prepared for administration of a patient, including, but not limited to, DUROS®-type dosage forms and dose-dumping.

Suitable vehicles that can be used to provide parenteral dosage forms as disclosed within are well known to those skilled in the art. Examples include, without limitation: sterile water; water for injection USP; saline solution; glucose solution; aqueous vehicles such as but not limited to, sodium chloride injection, Ringer's injection, dextrose Injection, dextrose and sodium chloride injection, and lactated Ringer's injection; water-miscible vehicles such as, but not limited to, ethyl alcohol, polyethylene glycol, and propylene glycol; and non-aqueous vehicles such as, but not limited to, corn oil, cottonseed oil, peanut oil, sesame oil, ethyl oleate, isopropyl myristate, and benzyl benzoate. Compounds that alter or modify the solubility of a pharmaceutically acceptable salt can also be incorporated into the parenteral dosage forms of the disclosure, including conventional and controlled-release parenteral dosage forms.

Pharmaceutical compositions can also be formulated to be suitable for oral administration, for example as discrete dosage forms, such as, but not limited to, tablets (including without limitation scored or coated tablets), pills, caplets, capsules, chewable tablets, powder packets, cachets, troches, wafers, aerosol sprays, or liquids, such as but not limited to, syrups, elixirs, solutions or suspensions in an aqueous liquid, a non-aqueous liquid, an oil-in-water emulsion, or a water-in-oil emulsion. Such compositions contain a predetermined amount of the pharmaceutically acceptable salt of the disclosed compounds, and may be prepared by methods of pharmacy well known to those skilled in the art. See generally, Remington: The Science and Practice of Pharmacy, 21st Ed., Lippincott, Williams, and Wilkins, Philadelphia Pa. (2005).

Conventional dosage forms generally provide rapid or immediate drug release from the formulation. Depending on the pharmacology and pharmacokinetics of the drug, use of conventional dosage forms can lead to wide fluctuations in the concentrations of the drug in a patient's blood and other tissues. These fluctuations can impact a number of parameters, such as dose frequency, onset of action, duration of efficacy, maintenance of therapeutic blood levels, toxicity, side effects, and the like. Advantageously, controlled-release formulations can be used to control a drug's onset of action, duration of action, plasma levels within the therapeutic window, and peak blood levels. In particular, controlled- or extended-release dosage forms or formulations can be used to ensure that the maximum effectiveness of a drug is achieved while minimizing potential adverse effects and safety concerns, which can occur both from under-dosing a drug (i.e., going below the minimum therapeutic levels) as well as exceeding the toxicity level for the drug. In some embodiments, the composition can be administered in a sustained release formulation.

In further embodiments, administration of a dual targeting siRNA agent is administered in combination an additional therapeutic agent. The dual targeting siRNA agent and an additional therapeutic agent can be administered in combination in the same composition, e.g., parenterally, or the additional therapeutic agent can be administered as part of a separate composition or by another method described herein.

The following non-limiting examples illustrate embodiments of the invention in operation. Having read the specification claims and Figures in their entirety, one skilled in the art will reasonably appreciate that numerous modifications and substitutions of the embodiments are possible and can be carried out without requiring undue experimentation. Such modifications and substitutions constitute part of the present invention.

### EXAMPLES

### Example 1: Identifying Target Gene with Multiple Target Regions

siRNA targeting sequences UBBs1 (SEQ ID NO: 96): AAGGCCAAGATCCAAGATAAA (U.S. Pat. No. 8,470,998) and novel molecule UBBs2 (SEQ ID NO: 97): AAGAGGTGGTATGCAGATCTT are useful as multi-targeting molecules according to certain embodiments of the instant invention. Utilizing Basic Local Alignment Search Tool (Blast) from the National Center for Biotechnology Information, UBB was found to have three potential targeting regions for UBBs1 with 19/19, 18/19, and 17/19 conserved identities. Based on this work in certain embodiments of the instant invention UBB is targeted by a siRNA that targets it in multiple internal regions.

### Example 2: Identification of a UBB Dual Target

After identifying a lead siRNA that bound to the UBB gene in three regions, 6 genes were identified through blast to have surprisingly conserved homology with UBB and thus to be a dual target partner for siRNA inhibition. These targets (DCP2, FAM83F, LOC646588, RNF17, NACA2, and UBC) were analyzed with the goal of finding key cancer dependencies. Analysis revealed that all but one were non-essential. UBC was found to be essential and a dual target of siRNA targeting UBBs1.

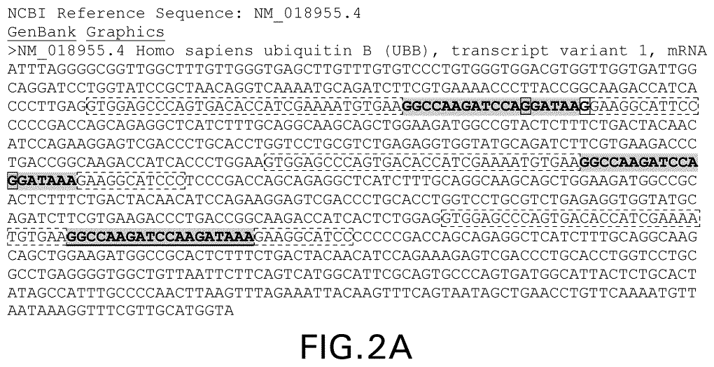

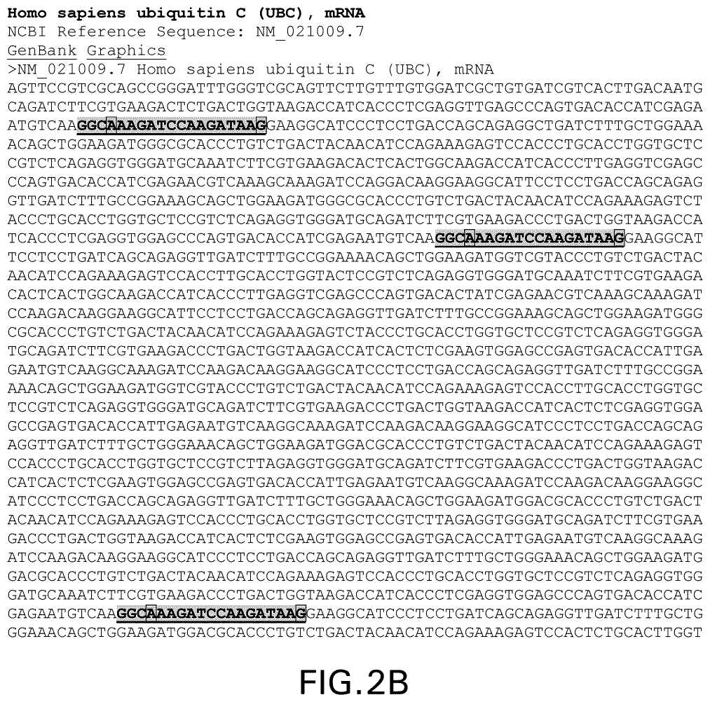

FIG. 2A: depicts the potential UBBs1 siRNA targeting sites (highlighted in yellow) on the UBB sequence. Nucleotide differences are highlighted in red and similar repeat sequences are in blue. FIG. 2B: depicts the potential UBBs1 siRNA targeting sites (highlighted in yellow) on the UBC sequence. Nucleotide differences are highlighted in red and similar repeat sequences are in blue.

### Example 3: Characterization of UBB/UBC Dual Inhibition

Analysis of UBC reveal three potential targeting regions for siRNA targeting UBBs1 all with 18/19 identify with a 14/14 identity stretch. Therefore, in certain embodiments one siRNA targets multiple genes and multiple regions within each gene (multi-multi-targeting). In certain embodiments of the instant invention UBB and UBC have been targeted multiple times each by the same siRNA.

### Example 4: A Dual UBB/UBC siRNA with EPCAM Aptamers

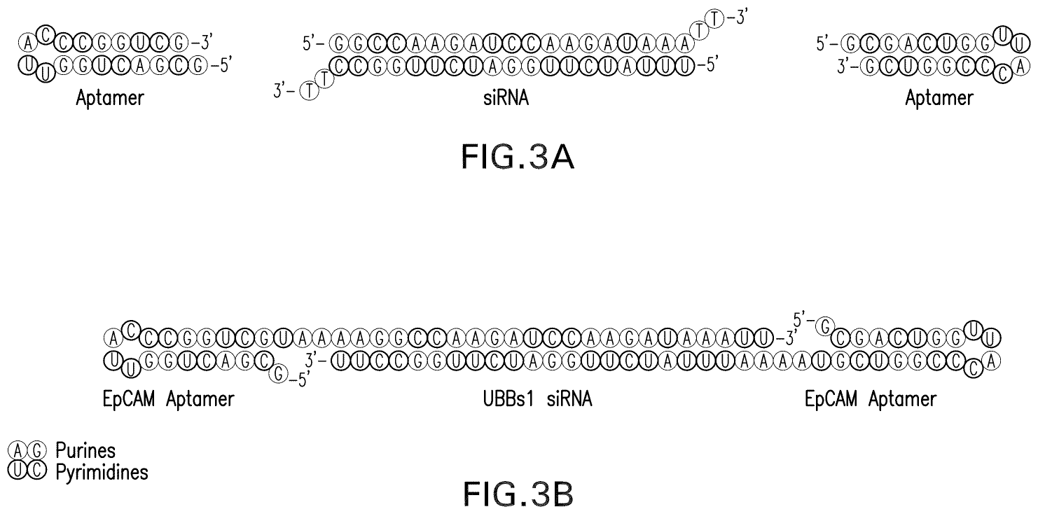

An aptamer-siRNA chimera with EPCAM aptamers and UBBs1 siRNA combined with an example of an acceptable linker, for example as disclosed in U.S. Pat. No. 10,960,086 (incorporated herein by reference in its entirety) is shown (FIG. 3B).

### Example 5: Library Development of UBB siRNA

A siRNA library was developed containing 19 compounds of 19 mer siRNA's targeting UBB Sequences:

(SEQ ID NO: 98):
5′-AAATGTGAAGGCCAAGATC-3′ (SO_U13)
(SEQ ID NO: 99):
5′-AATGTGAAGGCCAAGATCC-3′ (SO_U14)
(SEQ ID NO: 100):
5′-ATGTGAAGGCCAAGATCCA-3′ (SO_U15)
(SEQ ID NO: 101):
5′-TGTGAAGGCCAAGATCCAA-3′ (SO_U16)
(SEQ ID NO: 102):
5′-GTGAAGGCCAAGATCCAAG-3′(SO_U17)
(SEQ ID NO: 103):
5′-TGAAGGCCAAGATCCAAGA-3′(SO_U18)
(SEQ ID NO: 104):
5′-GAAGGCCAAGATCCAAGAT-3′ (SO_U19)
(SEQ ID NO: 105):
5′-AAGGCCAAGATCCAAGATA-3′(SO_U20)
(SEQ ID NO: 106):
5′-AGGCCAAGATCCAAGATAA-3′(SO_U21)
(SEQ ID NO: 107):
5′-GGCCAAGATCCAAGATAAA-3′(SO_U22)
(SEQ ID NO: 108):
5′-GCCAAGATCCAAGATAAAG-3′(SO_U23)
(SEQ ID NO: 109):
5′-CCAAGATCCAAGATAAAGA-3′(SO_U24)
(SEQ ID NO: 110):
5′-CAAGATCCAAGATAAAGAA-3′(SO_U25)
SEQ ID NO: 111):
5′-AAGATCCAAGATAAAGAAG-3′(SO_U26)
(SEQ ID NO: 112):
5′-AGATCCAAGATAAAGAAGG-3′(SO_U27)
(SEQ ID NO: 113):
5′-GATCCAAGATAAAGAAGGC-3′(SO_U28)
(SEQ ID NO: 114):
5′-ATCCAAGATAAAGAAGGCA-3′(SO_U29)
(SEQ ID NO: 115):
5′-TCCAAGATAAAGAAGGCAT-3′(SO_U30)
(SEQ ID NO: 116):
5′-CCAAGATAAAGAAGGCATC-3′(SO_U31)
(SEQ ID NO: 117):
5′-CAGGATCCTGGTATCCGCTAA-3′ (UBB_1)
(SEQ ID NO: 118):
5′-ATGGCATTACTCTGCACTATA-3′ (UBB_2)
(SEQ ID NO: 119):
5′-CCAACTTAAGTTTAGAAATTA-3′(UBB_3)
(SEQ ID NO: 120):
5′-GAGGCTCATCTTTGCAGGCAA-3′ (UBB_4)

Utilizing siRNA Wizard Software (InvivoGen), 6 scrambled UBB targeting sequences were developed as controls:

(SEQ ID NO: 121):
5′-GAACAACCGGCAAATAGAT-3′ (SO_U07)
(SEQ ID NO: 122):
5′-GCAATACGCGAAGACATAA-3′ (SO_U08)
(SEQ ID NO: 123):
5′-GAAAGACGGACCATAACAT-3′ (SO_U09)
(SEQ ID NO: 124):
5′-GAAGAACCACGAAGACTTA-3′ (SO_U10
(SEQ ID NO: 125):
5′-GTAGGACGCACAAACTAAA-3′ (SO_U11)
(SEQ ID NO: 126):
5′-GGACAGATCGCTAAACAAA-3′ (SO_U12)

Three UBB targeting compounds were developed including one (SEQ ID NO: 129) that was designed to target UBC in a conserved location to target both UBB and UBC.

(SEQ ID NO: 127):
5′-GGCCAAGATCCAGGATAAA -3′ (SO_U04)
(SEQ ID NO: 128):
5′-GGCCAAGATCCAGGATAAG-3′ (SO_U05)
(SEQ ID NO: 129):
5′-GGCAAAGATCCAAGATAAG-3′ (SO_U06)

### Example 6: In Vitro Validation of UBB siRNA Library

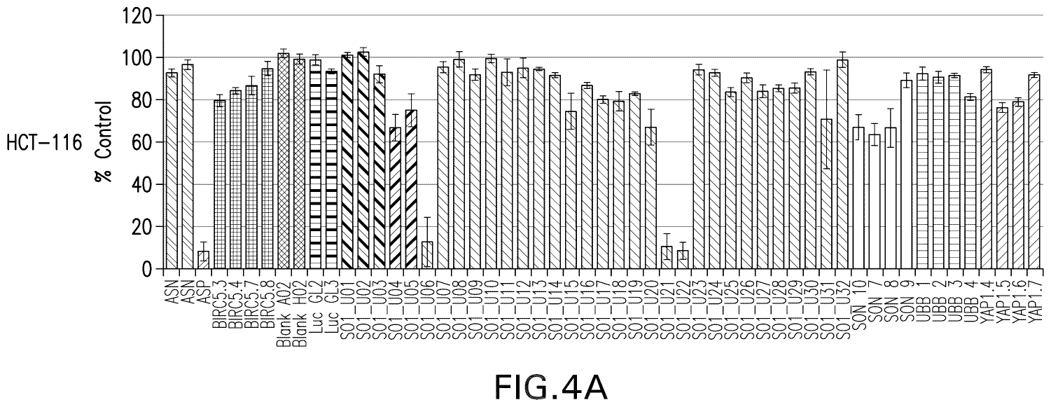

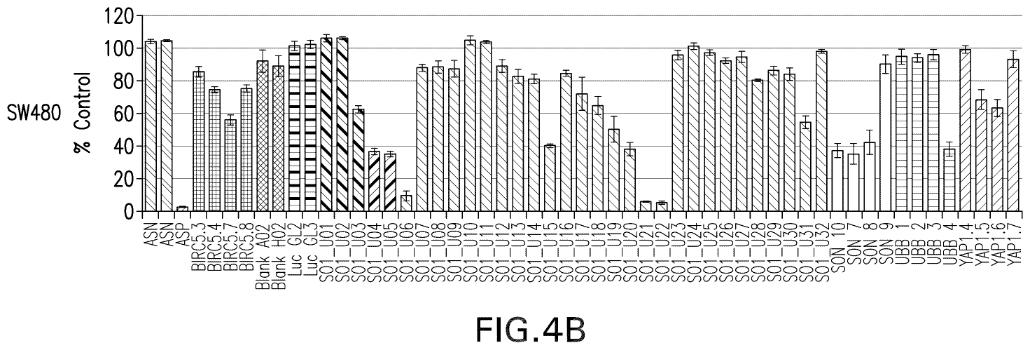

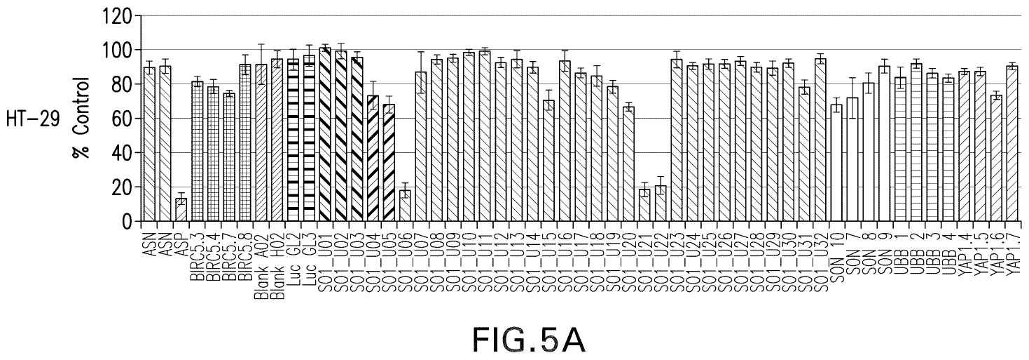

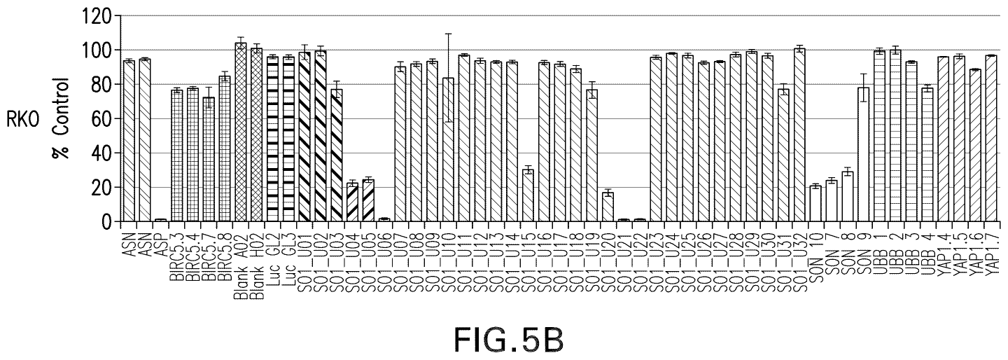

HCT-116, SW480, RKO, and HT-29 colon cancer cells were treated under standard siRNA transfection conditions with various siRNA compounds including those previously listed as well as ASN (negative control) and ASP (positive control) (16.7 nM; 96 hr) (FIGS. 4 and 5).

(SEQ ID NO: 316):
5′-AGGCCAAGAUCCAAGAUAA-3′ (U21)
(SEQ ID NO: 317):
5′-GGCCAAGAUCCAGGAUAAA-3′ (U04)
(SEQ ID NO: 318):
5′-GGCCAAGAUCCAGGAUAAG-3′ (U05)

Several siRNA targeting UBB are cytotoxic to SW480 and HCT-116 with novel siRNA (SEQ ID NO: 316) being the most potent. Other siRNA targeting UBB sequences (SEQ ID NO: 317) and (SEQ ID NO: 318) are cytotoxic but not as potent to UBB as the (SEQ ID NO: 316) siRNA. The siRNA targeting UBB like sequences on UBC (SEQ ID NO: 318) is as potent as the siRNA (SEQ ID NO: 316). A scrambled siRNA targeting sequence (SEQ ID NO: 124) does not have a cytotoxic effect and was used as a negative control.

### Example 7: In Vitro Validation of UBB siRNA Library

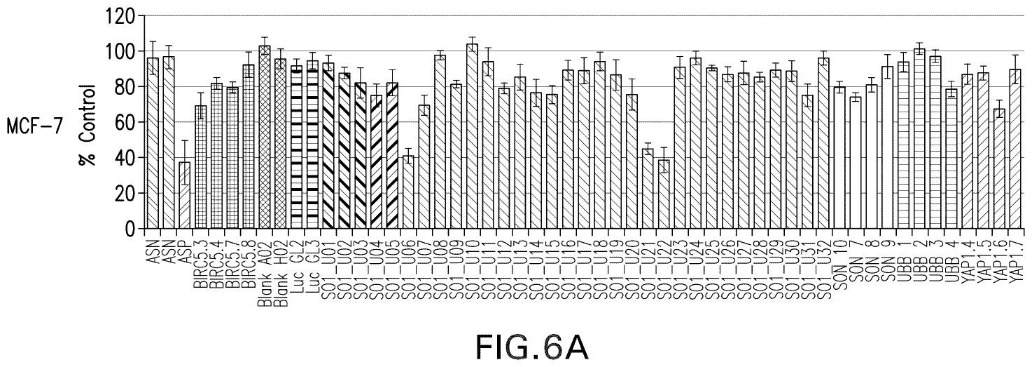

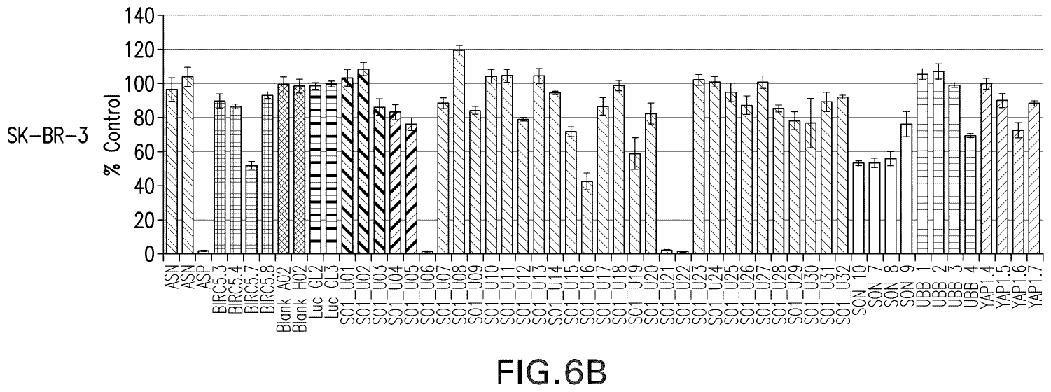

MCF-7 and SK-BR-3 breast cancer cells were treated under standard siRNA transfection conditions with various siRNA compounds including those previously listed as well as controls: ASN siRNA (negative), ASP siRNA (positive) (16.7 nM; 96 hr) (FIG. 6).

The siRNA targeting UBB (SEQ ID NO: 106) was cytotoxic to MCF-7 and SK-BR-3. The siRNA targeting (SEQ ID NO:129) was as potent as the siRNA to UBB (SEQ ID NO: 106). The novel siRNA targeting (SEQ ID NO: 106) is surprisingly more potent than known UBBs1 (SEQ ID NO: 107) while most of the siRNAs developed as disclosed in Example 5 were not cytotoxic (see FIGS. 4 through 7). This experiment demonstrated surprising efficacy of dual UBB and UBC siRNA inhibition on breast cancer cells.

### Example 8: Dose Response of Various siRNA Sequences on Colon Cancer Cells

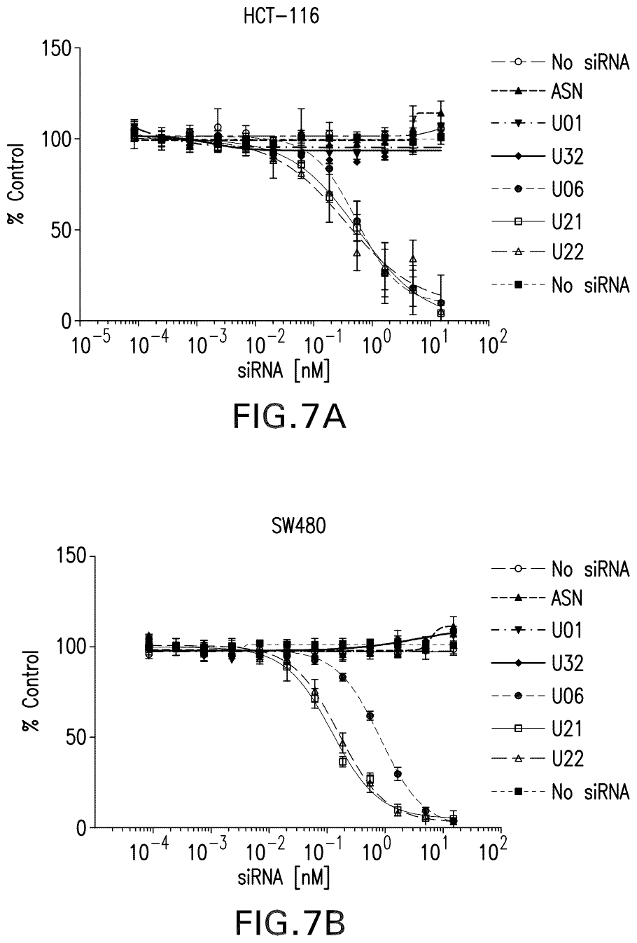

Dose response curve of HCT-116 and various siRNA sequences. Cells were grown to 2,000 cells/well in a 384-well plate, and treated with 62 pM-15 nM of compounds for 96 hours (FIG. 7A).

FIG. 7B: Dose response curve of SW480 and various siRNA sequences. Cells were grown to 2,000 cells/well in a 384-well plate, and treated with 62 pM-15 nM of compounds for 96 hours (FIG. 7B).

These results demonstrate that high concentrations of siRNA dual targeting molecules are not necessary for efficacy in cancer cell treatment.

### Example 9: Silencing of UBB and UBC

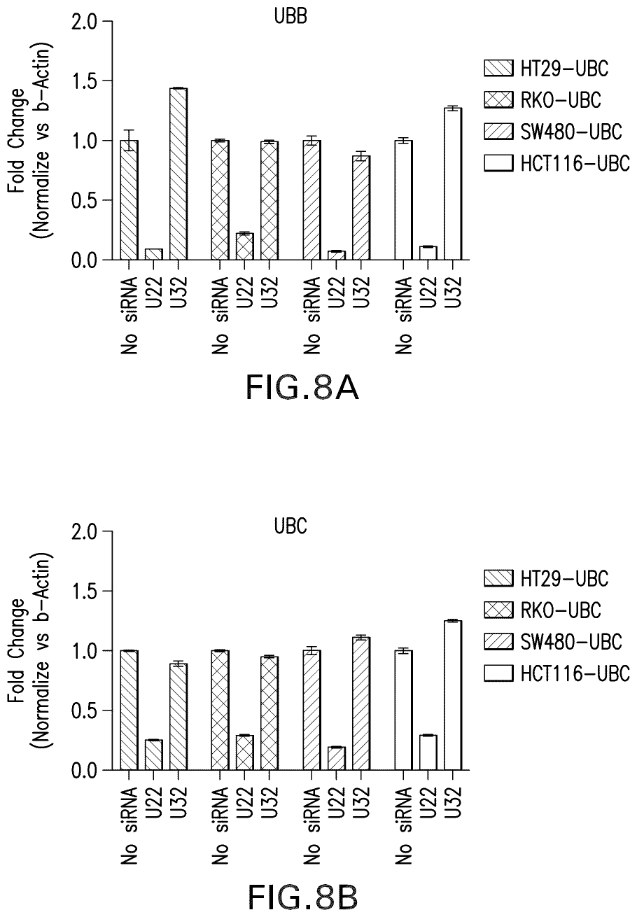

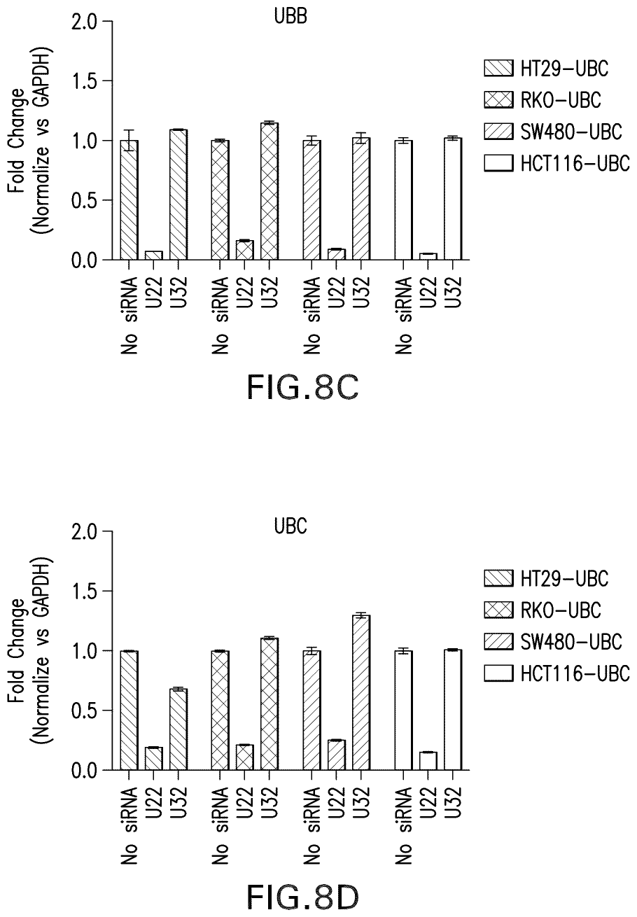

In order to demonstrate that active siRNA targeting (SEQ ID NO: 320), (SEQ ID NO: 316), and (SEQ ID NO: 319) silence both UBB and UBC and other UBB targeting siRNA's do not, a cell assay was performed using HT29, RKO, SW480, and HCT116 cells. Cells were treated with siRNA or control (15 nM siRNA; 20 hr). UBB (FIGS. 8A and 8C) or UBC levels (FIG. 8B or 8D) were measured and normalized by b-Actin (FIGS. 8A and 8B) or GAPDH (FIGS. 8C and 8D).

(SEQ ID NO: 319):
5′-GGCCAAGAUCCAAGAUAAA-3′ (U22)
(SEQ ID NO: 320):
5′-GGCAAAGAUCCAAGAUAAG-3′ (U06)

Results indicate the dual targeting capability of these siRNAs across multiple cell types.

### Example 10: UBB-UBC Expression in HCT116 Cells Following siRNA Knockdown

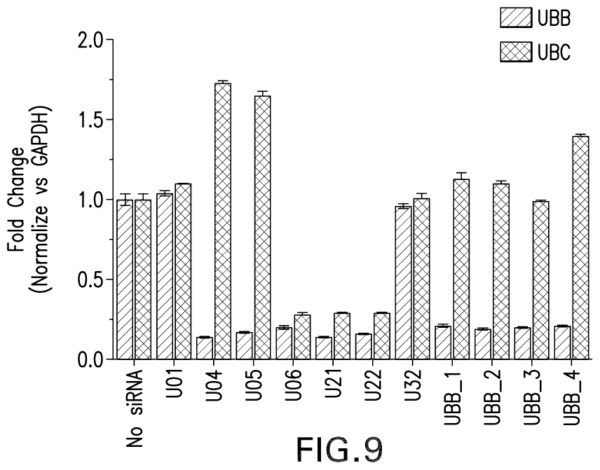

HCT116 cells were treated with the specified siRNA including U01, a Luciferase GL3 siRNA (15 nM siRNA; 20 hr). qPCR results were normalized to GAPDH. Results demonstrate the ability of siRNA's targeting (SEQ ID NO: 320), (SEQ ID NO: 316) and (SEQ ID NO: 319) to dual inhibit UBB and UBC. Control UBB inhibitors are not able to inhibit UBC (FIG. 9).

Additional UBB/UBC targeted siRNAs were developed and HCT116 cells were treated.

(SEQ ID NO: 130):
GCCGUACUCUUUCUGACUA (UBB_1G2)
(SEQ ID NO: 131):
GUAUGCAGAUCUUCGUGAA (UBB_2G2)
(SEQ ID NO: 132):
GACCAUCACUCUGGAGGUG (UBB_3G2)
(SEQ ID NO: 133):
CCCAGUGACACCAUCGAAA (UBB_4G2)
(SEQ ID NO: 134):
GUGAAGACCCUGACUGGUA (UBC_1G6)
(SEQ ID NO: 135):
AAGCAAAGAUCCAGGACAA (UBC_2G6)
(SEQ ID NO: 136):
GAAGAUGGACGCACCCUGU (UBC_3G6)
(SEQ ID NO: 137):
GUAAGACCAUCACUCUCGA (UBC_4G6)

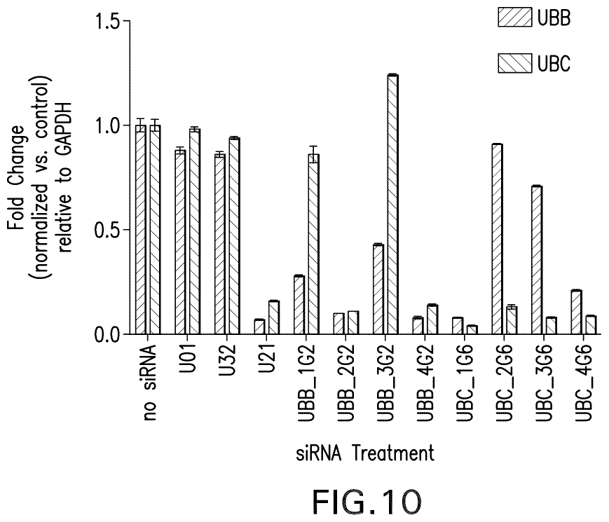

siRNA targeting (SEQ ID NO: 131) and (SEQ ID NO: 133) and (SEQ ID NO: 134) and (SEQ ID NO: 137) demonstrated significantly diminished UBB and UBC expression levels. (FIG. 10)

### Example 11

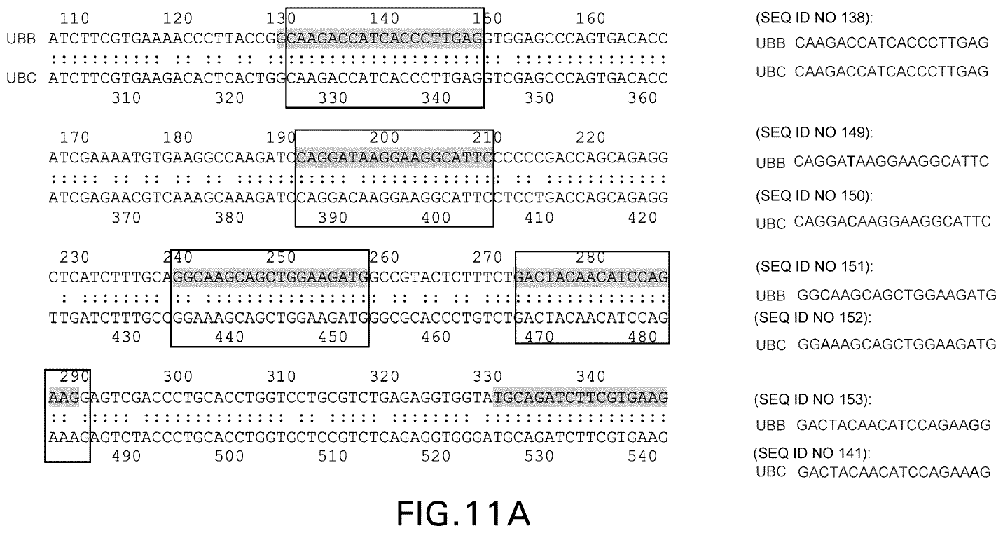

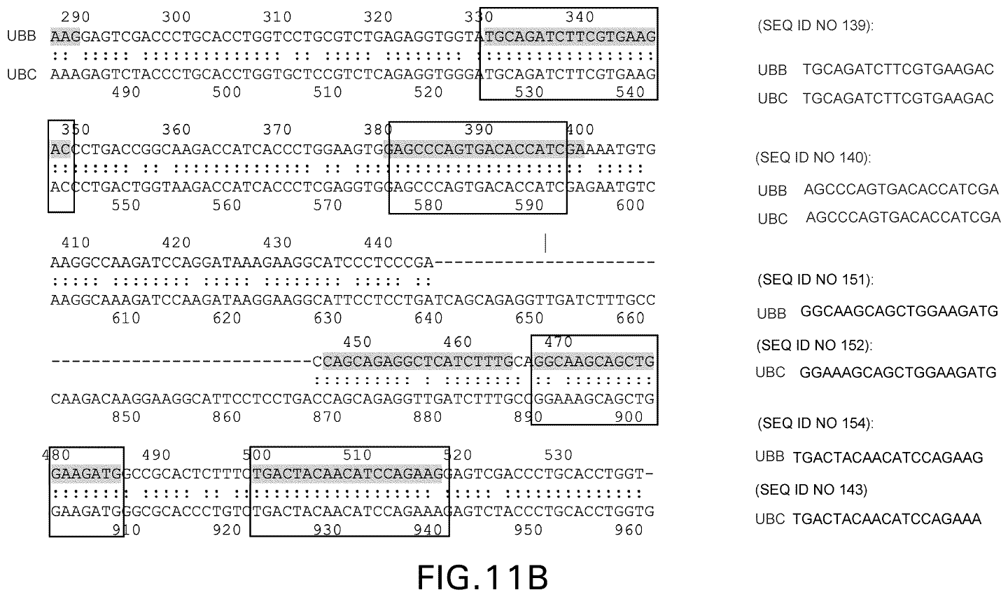

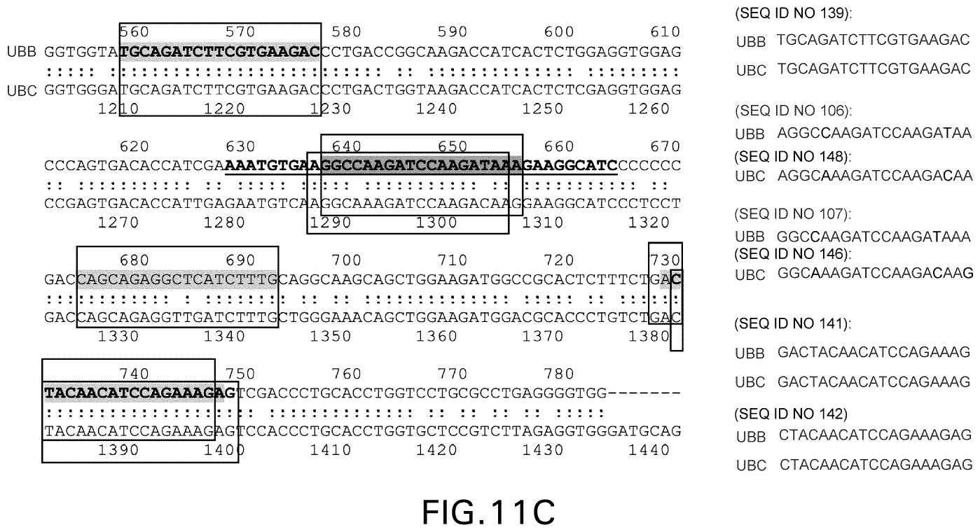

Homologous regions between UBB and UBC mRNA at regions with 19/19, 18/19 and 17/19 identity over the 19 nt stretch are shown (see also FIG. 11):

Dual UBB and UBC siRNA Targeting Sequences:

(SEQ ID NO: 138):
5′-CAAGACCATCACCCTTGAG-3′
(SEQ ID NO: 139):
5′-TGCAGATCTTCGTGAAGAC-3′
(SEQ ID NO: 140):
5′-AGCCCAGTGACACCATCGA-3′
(SEQ ID NO: 141):
5′-GACTACAACATCCAGAAAG-3′
(SEQ ID NO: 142):
5′-CTACAACATCCAGAAAGAG-3′
(SEQ ID NO: 143):
5′-TGACTACAACATCCAGAAA-3′

UBB Similar to UBC siRNA Targeting Sequences:

(SEQ ID NO: 144):
5′-AGTGACACCATCGAAAATG-3′
(SEQ ID NO: 145):
5′-AGGCAAAGATCCAAGATAA-3′
(SEQ ID NO: 146):
5′-GGCAAAGATCCAAGACAAG-3′
(SEQ ID NO: 147):
5′-CAAGGCAAAGATCCAAGAC-3′
(SEQ ID NO: 148):
5′-AGGCAAAGATCCAAGACAA-3′
(SEQ ID NO: 149):
5′-CAGGATAAGGAAGGCATTC-3′
(SEQ ID NO: 150):
5′-CAGGACAAGGAAGGCATTC-3′
(SEQ ID NO: 151):
5′-GGCAAGCAGCTGGAAGATG-3′
(SEQ ID NO: 152):
5′-GGAAAGCAGCTGGAAGATG-3′
(SEQ ID NO: 153):
5′-GACTACAACATCCAGAAGG-3′
(SEQ ID NO: 154):
5′-TGACTACAACATCCAGAAG-3′

(SEQ ID NO: 138) was identified 2× in UBC and 1× in UBB. (SEQ ID NO: 139) was identified 4× in UBC and 1× in UBB. (SEQ ID NO: 140) was identified 2× in UBC and 3× in UBB. (SEQ ID NO: 141) was identified 7× in UBC and 1× in UBB. (SEQ ID NO: 142) was identified 7× in UBC and 1× in UBB.

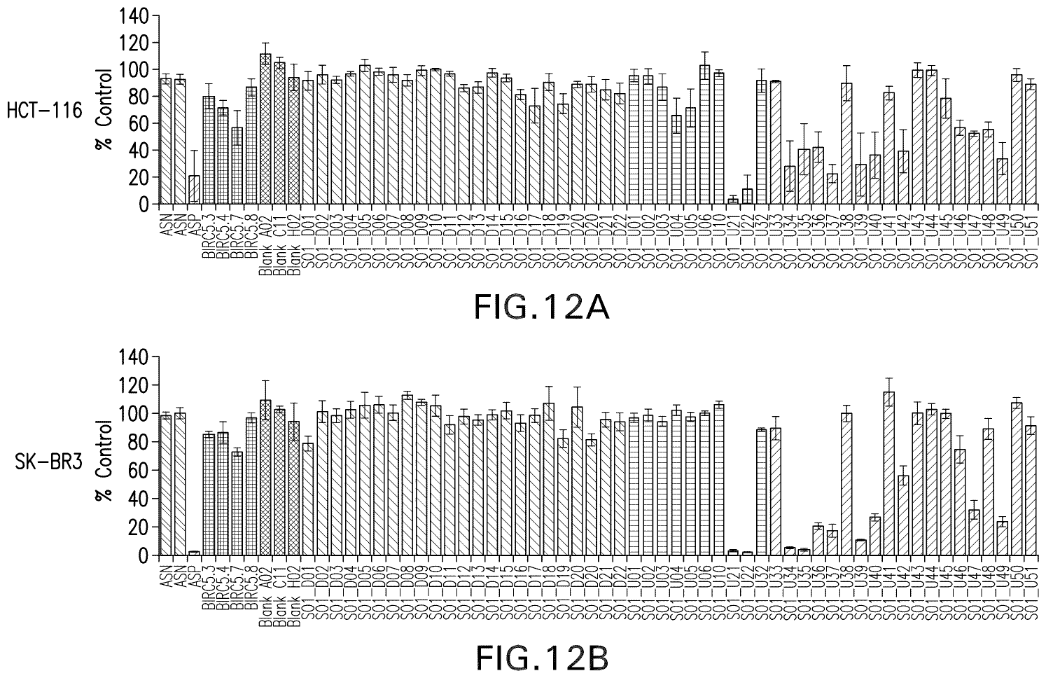

HCT-116 (FIG. 12a), a colon cancer cell line, and SK-BR3 (FIG. 12b), a breast cancer cell line, were treated under standard siRNA transfection conditions with siRNA compounds targeting mRNA sequences above as well as ASN (negative control) and ASP (positive control) (16.7 nM; 96 hr). U32, U50, U51 are negative control siRNAs.

(SEQ ID NO: 321):
5′-UGCAGAUCUUCGUGAAGAC-3′ (U34)
(SEQ ID NO: 322):
5′-AGCCCAGUGACACCAUCGA-3′ (U35)
(SEQ ID NO: 323):
5′-GACUACAACAUCCAGAAAG-3′ (U36)
(SEQ ID NO: 324):
5′-CUACAACAUCCAGAAAGAG-3′ (U37)
(SEQ ID NO: 325):
5′-AGUGACACCAUCGAAAAUG-3′ (U38)

These results identify (SEQ ID NO: 321), (SEQ ID NO: 322), (SEQ ID NO: 323), (SEQ ID NO: 324), and (SEQ ID NO: 325) as siRNA targets where the siRNA will have the ability to inhibit both UBB and UBC, although none are as potent as SEQ ID NO: 316 or 319.

### Example 12: UBB-UBC Species

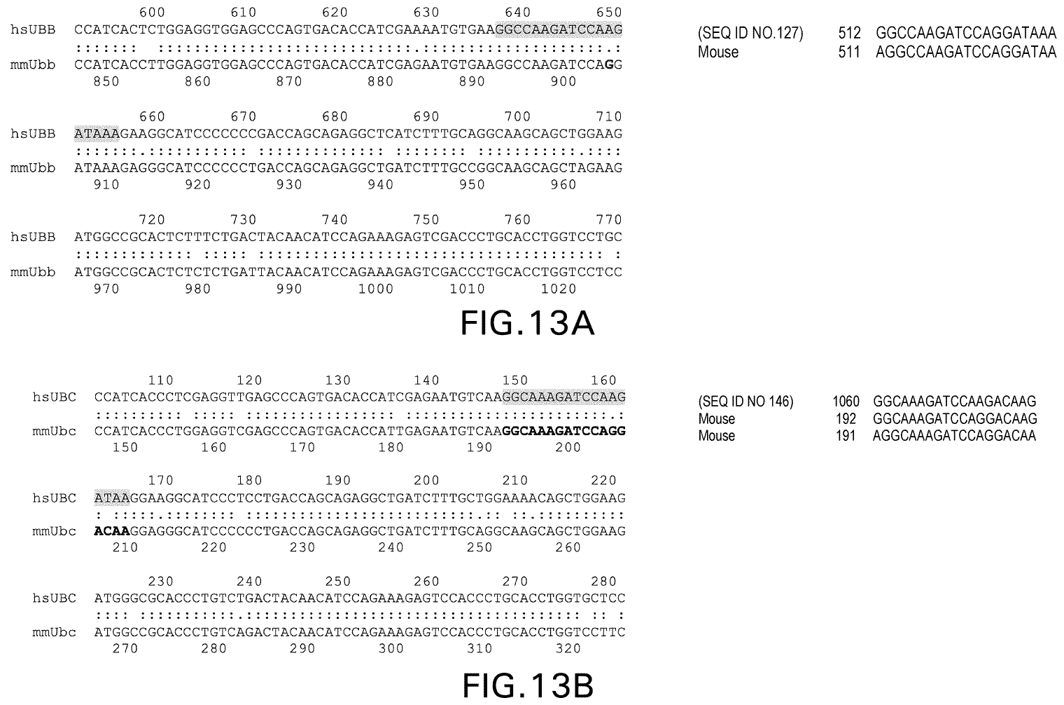

Human UBB and UBC sequences were compared to mouse in order to find potential homologous regions for in vivo work in therapeutic drug development. Of the previously identified siRNAs, the (SEQ ID NO: 127) and (SEQ ID NO: 146) sequences were found to be effective siRNA targeting regions for human that contain high homology to mouse. (SEQ ID NO: 127) with minimal nucleotide differences was identified 4× in mouse UBB and (SEQ ID NO: 146) with minimal nucleotide differences was identified 9× in mouse UBC. These sequences are useful for multi-species in vivo studies. (FIG. 13)

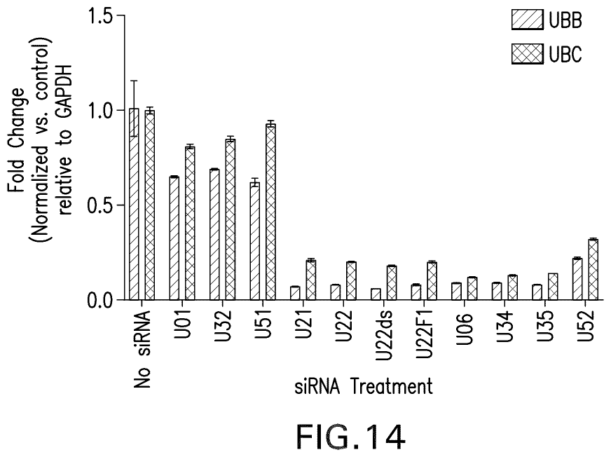

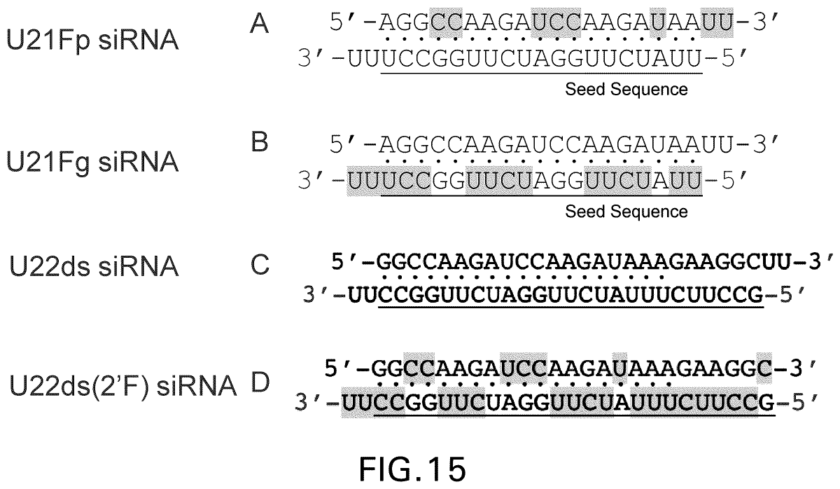

One mouse sequence (SEQ ID NO: 155: U52) was useful to inhibit both UBB and UBC (FIG. 14). Additionally, a dicer substrate siRNA (SEQ ID NO: 156: U22ds (FIG. 15C)) and a 2′F pyrimidine modified siRNA (SEQ ID NO: 316: U21F) were included in this experiment. Gene expression of HCT116 cells was measured by qPCR following siRNA treatment and these siRNAs were found to effectively decrease expression of both UBB and UBC (FIG. 14).

(SEQ ID NO: 155):
5′-GGCAAAGAUCCAGGACAAG-3 (U52)
(SEQ ID NO: 156):
5′-GGCCAAGAUCCAAGAUAAAGAAGGC-3′ (U22ds)

### Example 13: UBB-UBC Modifications

2′F pyrimidine modifications of the siRNA targeting SEQ ID NO: 316 are depicted in FIG. 14. The modifications can either be on the passenger strand, U21Fp (FIG. 15A) or the guide strand, U21Fg (FIG. 15B). The guide strand is underlined. A dicer substrate as well as a 2′F modified dicer substrate are also provided (FIGS. 15C and D) (SEQ ID NO: 156).

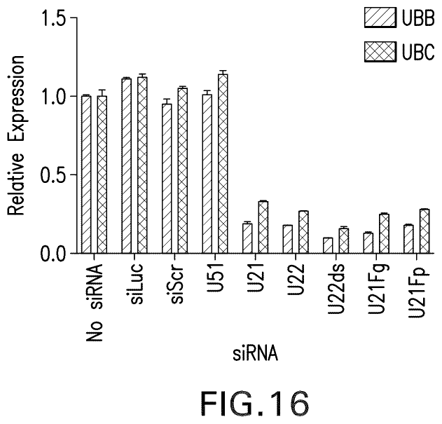

HCT-116 cells were treated with UBB-UBC targeting siRNAs. Modified and unmodified versions of SEQ ID NO: 316 are able to silence UBB and UBC with similar activity to unmodified (FIG. 16).

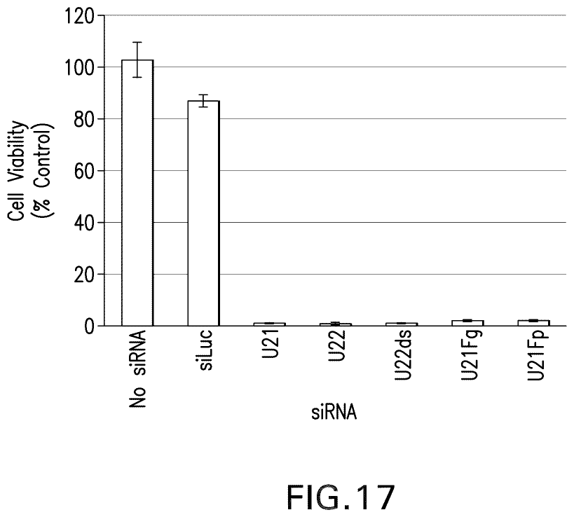

Cell viability was measured, and the silencing of these genes demonstrated >98% cytotoxicity at 96 hours (FIG. 17)

### Example 14: Multi-Targeting Domains

Novel sequences were identified with highly conserved homology useful for dual or triple targeting.

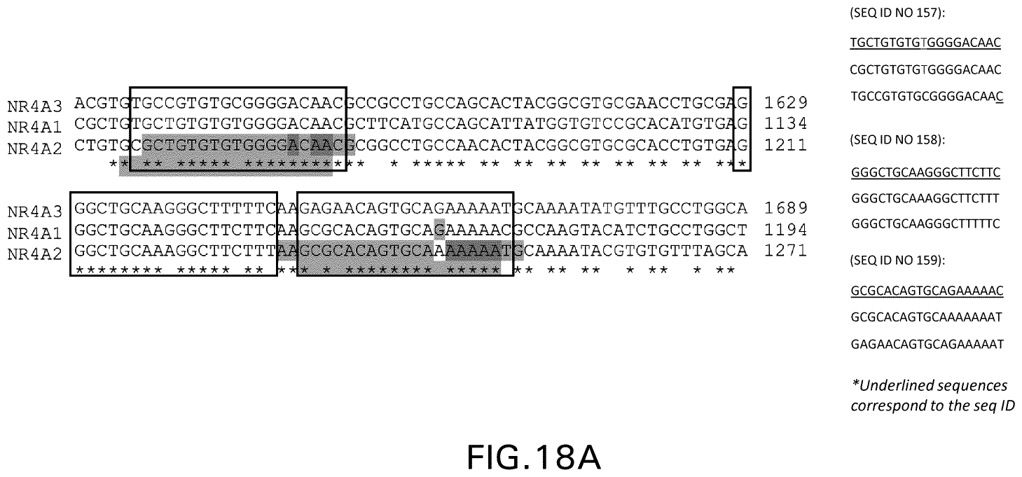

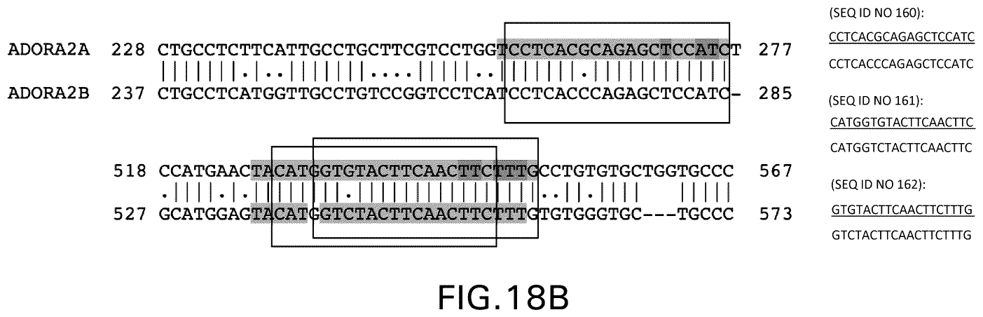

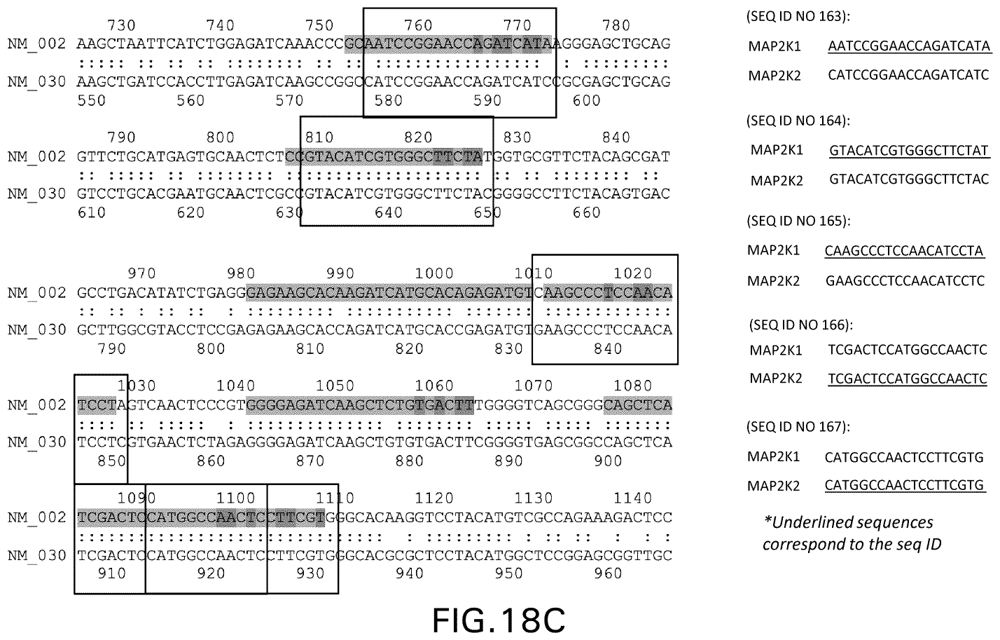

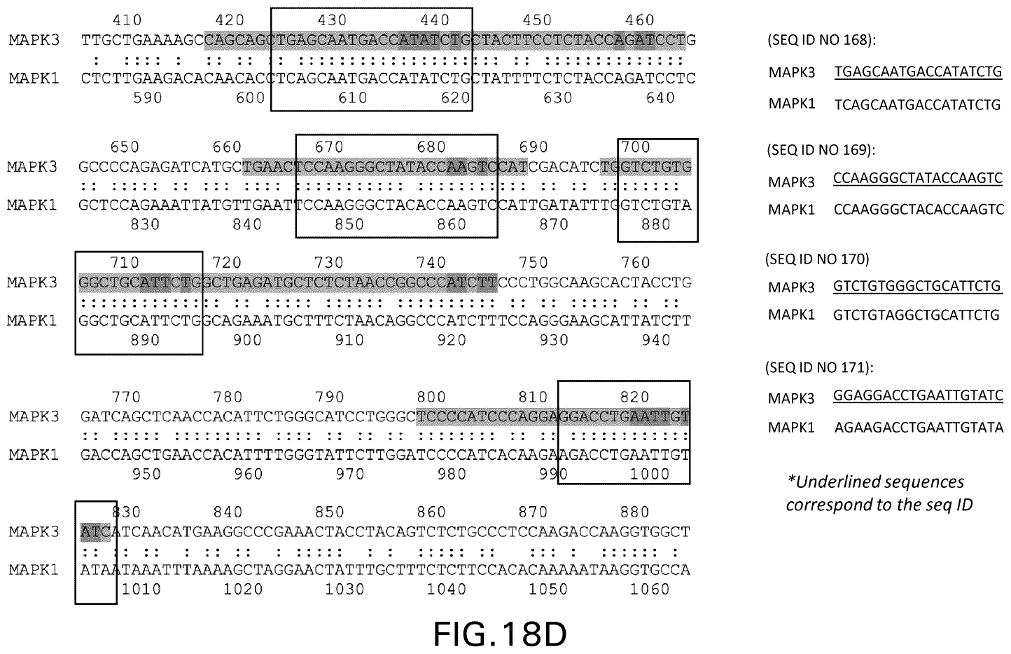

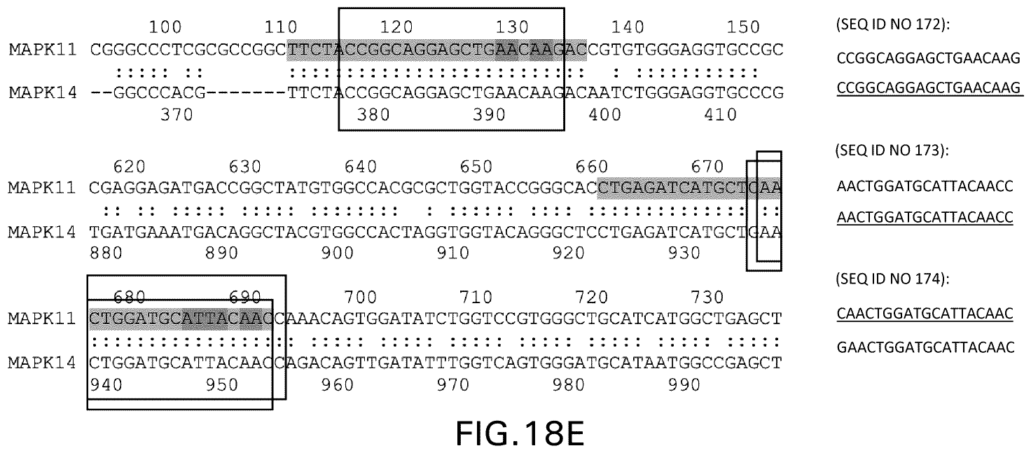

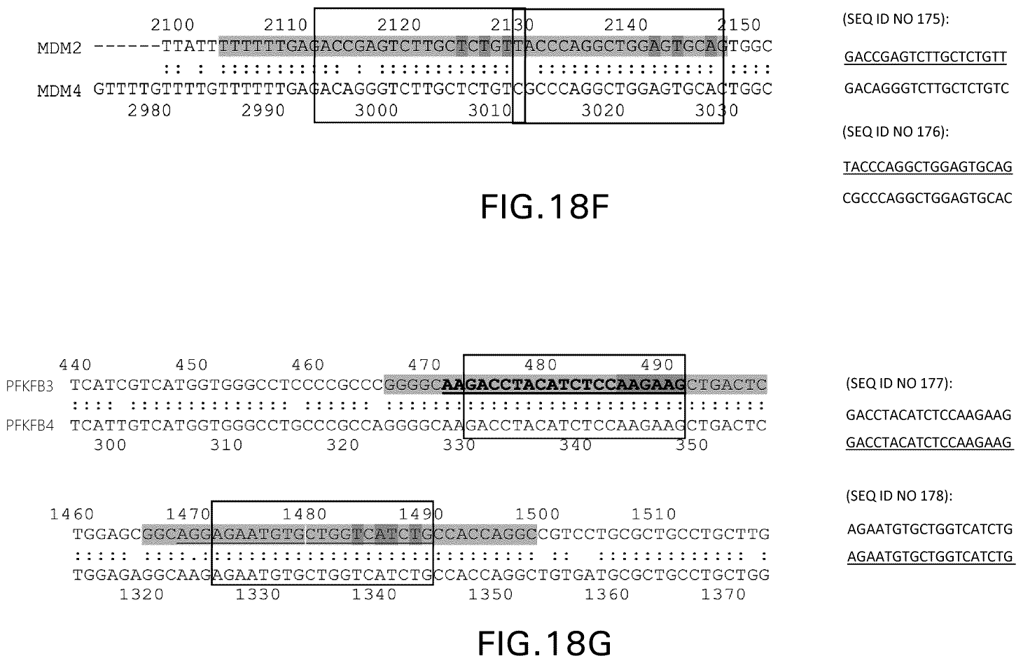

NR4A3 was found to have three targeting regions which have 18/19 conserved identities across all three sequences with NR4A1, and 18/19, 18/19, and 17/19 conserved identities with NR4A2 (FIG. 18A).

NR4A1, NR4A2, and NR4A3 siRNA Targeting Sequences:

(SEQ ID NO: 157):
5′-TGCTGTGTGTGGGGACAAC-3′
(SEQ ID NO: 158):
5′-GGGCTGCAAGGGCTTCTTC-3′
(SEQ ID NO: 159):
5′-GCGCACAGTGCAGAAAAAC-3′

ADORA2A was found to have three targeting regions which have 18/19 conserved identities across all three sequences with ADORA2B (FIG. 18B).

ADORA2A and ADORA2B siRNA Targeting Sequences:

(SEQ ID NO: 160):
5′-CCTCACGCAGAGCTCCATC-3′ (D04)
(SEQ ID NO: 161):
5′-CATGGTGTACTTCAACTTC-3′ (D05)
(SEQ ID NO: 162):
5′-GTGTACTTCAACTTCTTTG-3′ (D06)

MAP2K1 was found to have five targeting regions which have 19/19, 19/19, 17/19, 18/19, and 17/19 conserved identities with MAP2K2 (FIG. 18C).

MAP2K1 and MAP2K2 siRNA Targeting Sequences:

(SEQ ID NO: 163):
5′-AATCCGGAACCAGATCATA-3′ (D07)
(SEQ ID NO: 164):
5′-GTACATCGTGGGCTTCTAT-3′ (D08)
(SEQ ID NO: 165):
5′-CAAGCCCTCCAACATCCTA-3′ (D09)
(SEQ ID NO: 166):
5′-TCGACTCCATGGCCAACTC-3′ (D10)
(SEQ ID NO: 167):
5′-CATGGCCAACTCCTTCGTG-3′ (D11)

ERK1 (MAPK3) was found to have four targeting regions which have 18/19, 18/19, 16/19 conserved identities with ERK2 (MAPK1) (FIG. 18D).
MAPK3 and MAPK1 siRNA Targeting Sequences:

(SEQ ID NO: 168):
5′-TGAGCAATGACCATATCTG-3′ (D12)
(SEQ ID NO: 169):
5′-CCAAGGGCTATACCAAGTC-3′ (D13)
(SEQ ID NO: 170):
5′-GTCTGTGGGCTGCATTCTG-3′ (D14)
(SEQ ID NO: 171):
5′-GGAGGACCTGAATTGTATC-3′ (D15)

MAPK11 was found to have three targeting regions which have 19/19, 19/19, and 18/19 conserved identities with MAPK14 (FIG. 18E).

MAPK11 and MAPK14 siRNA Targeting Sequences:

(SEQ ID NO: 172):
5′-CCGGCAGGAGCTGAACAAG-3′ (D16)
(SEQ ID NO: 173):
5′-AACTGGATGCATTACAACC-3′ (D17)
(SEQ ID NO: 174):
5′-CAACTGGATGCATTACAAC-3′ (D18)

MDM2 was found to have two targeting regions which have 16/19 and 16/19 conserved identities with MDM4 (FIG. 18F).

MDM2 and MDM4 siRNA Targeting Sequences:

(SEQ ID NO: 175):
5′-GACCGAGTCTTGCTCTGTT-3′
(SEQ ID NO: 176):
5′-TACCCAGGCTGGAGTGCAG-3′

PFKFB3 was found to have two targeting regions which both had 19/19 conserved identities with PFKFB4 (FIG. 18G).

PFKFB3 and PFKFB4 siRNA Targeting Sequences:

(SEQ ID NO: 177):
5′-GACCTACATCTCCAAGAAG-3′
(SEQ ID NO: 178):
5′-AGAATGTGCTGGTCATCTG-3′

### Example 15: siRNA Target Validation—Multiple Gene Expression Following siRNA Treatment

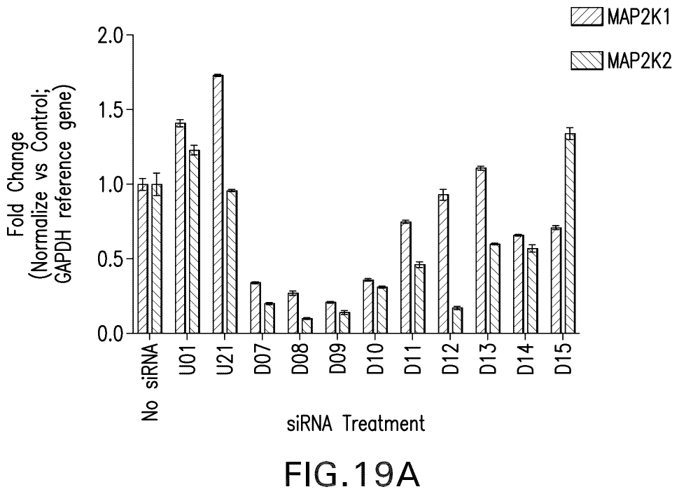

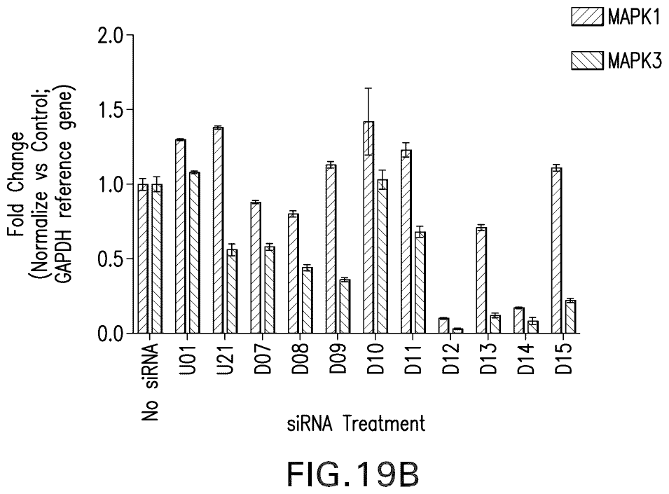

HCT116 cells were treated with siRNA and the expression levels of MAP2K1 and MAP2K2 (FIG. 19A) and MAPK1 and MAPK3 were measured (FIG. 19B).

SiRNA targeting sequences (SEQ ID NO: 163-165) reduced MAP2K1 and MAP2K2 expression. SiRNA targeting sequences (SEQ ID NO: 168-170) effectively reduced expression of MAPK1 and MAPK3. The siRNA targeting sequence (SEQ ID NO: 168) knocked down expression of MAPK1, MAPK3, and MAP2K2.

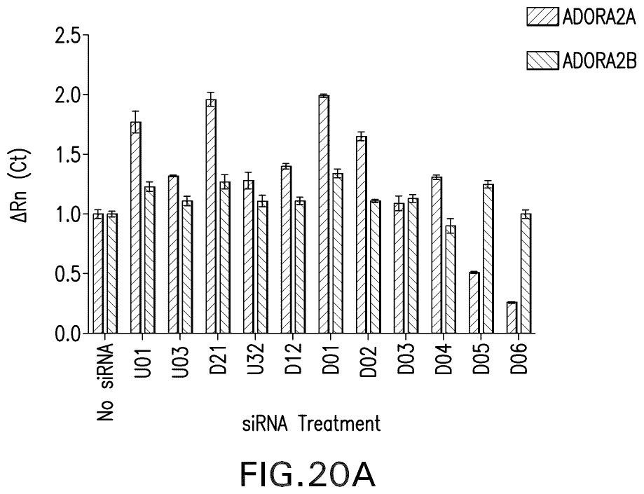

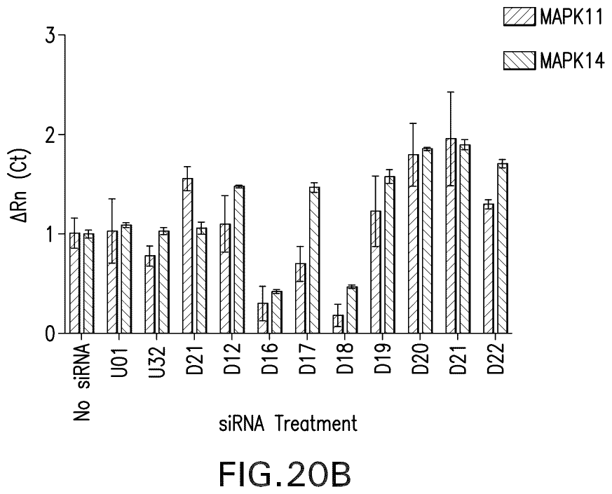

SKBR3 cells were treated with siRNA and the expression levels of ADORA2A/ADORA2B (FIG. 20A) were measured. siRNA targeting (SEQ ID NO: 161) and (SEQ ID NO: 162) demonstrated the largest decrease in ADORA2A expression.

HCT116 cells were treated with siRNA and the expression levels of MAPK11/MAPK14 (FIG. 20B) were measured. siRNA targeting (SEQ ID NO: 172) and (SEQ ID NO: 174) targeting siRNA demonstrates efficacy in decreasing expression of MAPK11 and MAPK14.

### Example 16: siRNA Target Validation-Gene Expression Reduction Following siRNA Treatment

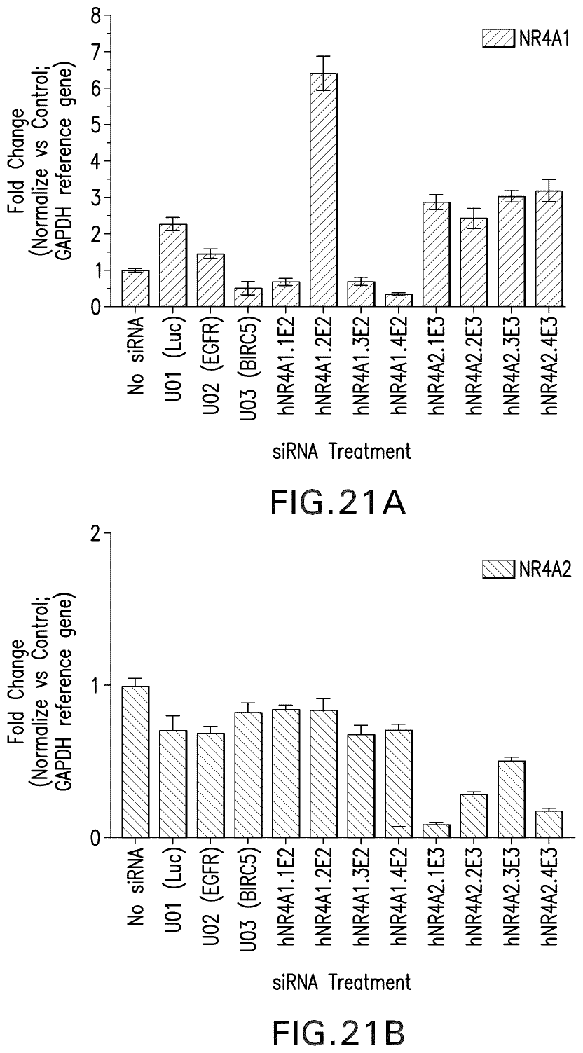

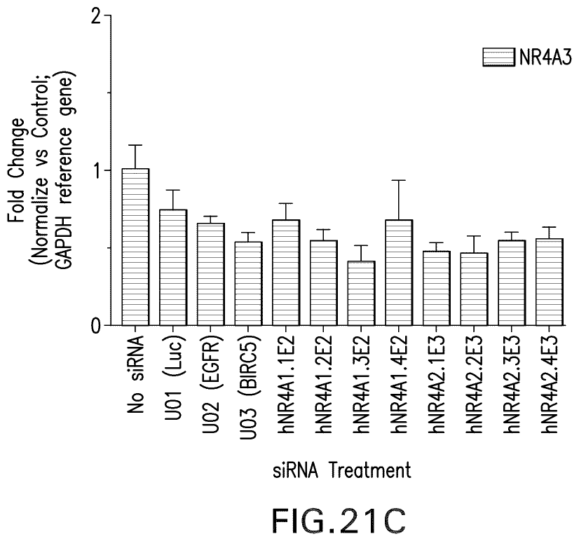

SK-BR3 cells were treated with siRNA and the expression of NR4A1 (FIG. 21A), NR4A2 (FIG. 21B), and NR4A3 (FIG. 21C) was measured after treatment.

### Target Sequences of NR4A1:

(SEQ ID NO: 179):
5′-GCACCTTCATGGACGGCTA-3′
(hNR4A1.1E2)
(SEQ ID NO: 180):
5′-GCATTATGGTGTCCGCACA-3′
(hNR4A1.2E2)
(SEQ ID NO: 181):
5′-TGAAGGAAGTTGTCCGAAC-3′
(hNR4A1.3E2)
(SEQ ID NO: 182):
5′-CTGCAGAACCGCATCGCCA-3′
(hNR4A1.4E2)

### Target Sequences of NR4A2:

(SEQ ID NO: 183):
5′-CCACGTGACTTTCAACAAT-3′
(hNR4A2.1E3)
(SEQ ID NO: 184):
5′-ACATTCAGATGCACAACTA-3′
(hNR4A2.2E3)
(SEQ ID NO: 185):
5′-GGACAAGCGTCGCCGGAAT-3′
(hNR4A2.3E3)
(SEQ ID NO: 186):
5′-CCACCTTGCTTGTACCAAA-3′
(hNR4A2.4E3)

siRNA targeting (SEQ ID NO: 180) induced NR4A1 expression while (SEQ ID NO: 179), (SEQ ID NO: 181) and (SEQ ID NO: 182) reduced it. All four siRNAs targeting NR4A2 sequences reduced NR4A2 expression with (SEQ ID NO: 183) decreasing expression 91%. Sequences reduced NR4A3 expression.

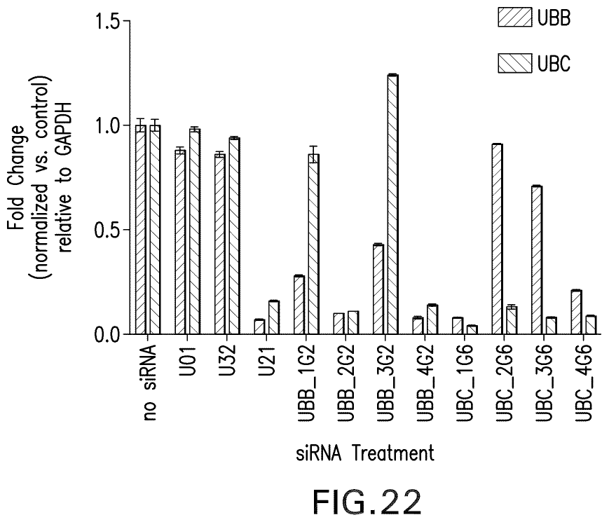

HCT116 cells were treated with siRNA and the expression of UBB and UBC (FIG. 22) was measured after treatment.

### Target Sequences of UBB:

(SEQ ID NO: 130):
5′-GCCGUACUCUUUCUGACUA-3′
(UBB_1G2)
(SEQ ID NO: 131):
5′-GUAUGCAGAUCUUCGUGAA-3′
(UBB_2G2)
(SEQ ID NO: 132):
5′-GACCAUCACUCUGGAGGUG-3′
(UBB_3G2)
(SEQ ID NO: 133):
5′-CCCAGUGACACCAUCGAAA-3′
(UBB_4G2)

### Target Sequences of UBC:

(SEQ ID NO: 134):
5′-GUGAAGACCCUGACUGGUA-3′
(UBC_1G6)
(SEQ ID NO: 135):
5′-AAGCAAAGAUCCAGGACAA-3′
(UBC_2G6)
(SEQ ID NO: 136):
5′-GAAGAUGGACGCACCCUGU-3′
(UBC_3G6)
(SEQ ID NO: 137):
5′-GUAAGACCAUCACUCUCGA-3′
(UBC_4G6)

All four siRNAs targeting UBB alone demonstrated reduction in UBB expression, with (SEQ ID NO: 131) and (SEQ ID NO: 133) demonstrating significant reduction in expression. All four siRNAs targeting UBC demonstrated significant decreases in expression levels of UBC. (SEQ ID NO: 189), (SEQ ID NO: 133), and (SEQ ID NO: 134) demonstrated comparable dual action inhibition to U21.

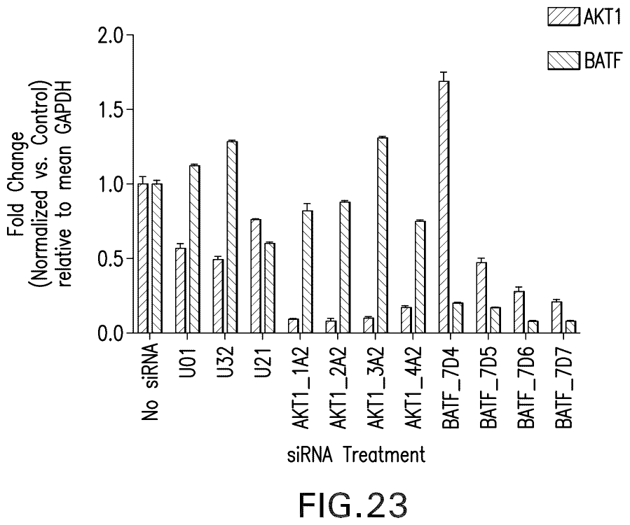

SKBR3 cells were treated with siRNA and the expression of AKT1 and BATF (FIG. 23) was measured after treatment.

### Target Sequences of AKT1:

(SEQ ID NO: 187):
5′-GACAAGGACGGGCACAUUA-3′
(AKT1_1A2)
(SEQ ID NO: 188):
5′-GCUACUUCCUCCUCAAGAA-3′
(AKT1_2A2)
(SEQ ID NO: 189):
5′-GACCGCCUCUGCUUUGUCA-3′
(AKT1_3A2)
(SEQ ID NO: 190):
5′-GGCAGCACGUGUACGAGAA-3′
(AKT1_4A2)

### Target Sequences of BATF:

(SEQ ID NO: 191):
5′-GUACAGCGCCCACGCAUUC-3′
(BATF_7D4)
(SEQ ID NO: 192):
5′-GAAACAGAACGCGGCUCUA-3′
(BATF_7D5)
(SEQ ID NO: 193):
5′-GAACGCGGCUCUACGCAAG-3′
(BATF_7D6)
(SEQ ID NO: 194):
5′-AGAGUUCAGAGGAGGGAGA-3′
(BATF_7D7)

All four siRNAs targeting AKT1 alone demonstrated reduction in AKT1 expression, with minimal off-target effects. All four BATF targeting siRNAs exhibited significant reduction in BATF expression with dual action inhibition to AKT1.

### Example 17: EPCAM Aptamer Construction

EpCAM aptamers were individually synthesized by in vitro transcription with PCR products as templates. The ssDNA of EpCAM aptamer containing T7 RNA polymerase promoter site (underlined) and adaptor sequence (5′-TAATACGACTCACTATAGCGACTGGTTACCCGGTCGT-3′) (SEQ ID NO: 195) was synthesized from IDT as a PCR template. PCR was performed with forward primer (5′-TAATACGACTCACTATA GCGACTGGTTA-3) (SEQ ID NO: 196) and reverse primer (5′-ACGACCGGGTAACCAGTCGC-3′) (SEQ ID NO: 197). The PCR products were put into T-A cloning pCR 2.1 vector (Invitrogen) and sequenced. Transcription was performed with PCR product as templates using DuraScript transcription kits following manufacture's instruction.

### Example 18: Bivalent Aptamer-Driven Delivery of Two siRNAs

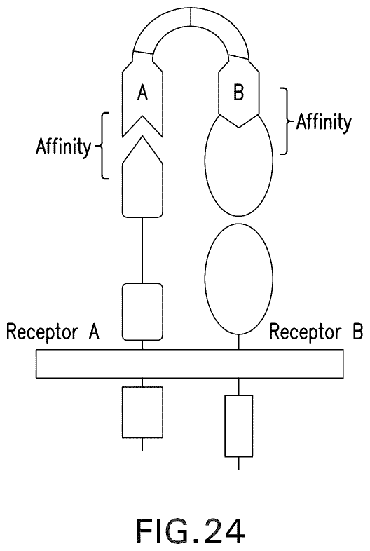

Bivalent aptamers support increased cargo internalization and specificity. Moreover, proof of concept experiments for increasing ligand valency to augment cargo delivery has been demonstrated by the use of nanoparticle-based carriers (Pardella et al., Cancers 2020, 12(10), 2799) (FIG. 24).

### Example 19: EPCAM-UBB Aptamer-siRNA Chimera Construction

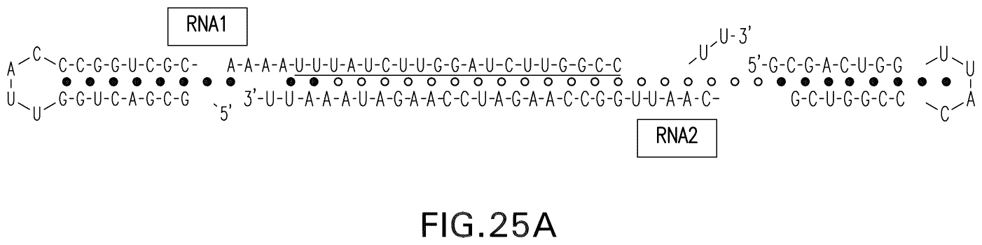

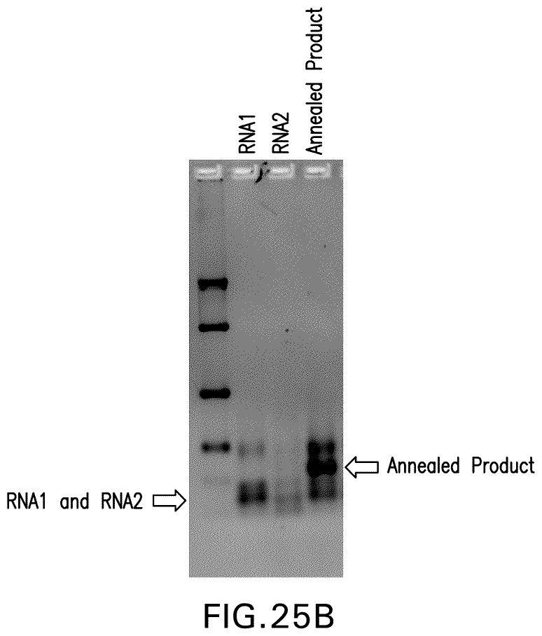

EpCAM-directed aptamers-siRNA chimeras were individually synthesized by in vitro transcription from an annealed DNA templates (FIG. 25A). For RNA 1, two ssDNA containing T7 RNA polymerase promoter site (underlined) and adaptor sequence

(SEQ ID NO: 198)
(5′-GTAATACGACTCACTATAGGCGACTGGTTACC
CGGTCGCAATTGGCCAAGATCCAAGATAAATT-3′)har
and
(SEQ ID NO: 199)
(5′-AATTTATCTTGGAUCTTGGCCAATTGCGACCGG
GTAACCAGTCGCCTAATACGACTCACTATA3′)

were synthesized by IDT as a T7 template. For RNA 2, two ssDNA containing T7 RNA polymerase promoter site (underlined) and adaptor sequence

(SEQ ID NO: 200)
(5′-GTAATACGACTCACTATAGGCGACTGGTTACCC
GGTCGCAAAATTTATCTTGGATCTTGGCCTT-3′)
and
(SEQ ID NO: 201)
(5′-AAGGCCAAGATCCAAGATAAATTTTGCGACCGG
GTAACCAGTCGCCTATAGTGAGTCGTATTAC-3′)

were synthesized by IDT as a T7 template. The annealed double stranded DNA for each RNA1 and RNA2 were used as templates for T7 polymerase using DuraScript transcription kits following manufacture's instruction. The two RNAs were further purified and mixed at molar ratio 1:1 and annealed to form the chimeric molecule by heating at 94° C. for 3 min followed by slowly cooling to room temperature within 1 h. Resulting products were run on a gel for confirmation (FIG. 25B).

### Example 20: HER3-U21(UBB)-HER2 Aptamer-siRNA Chimera Construction

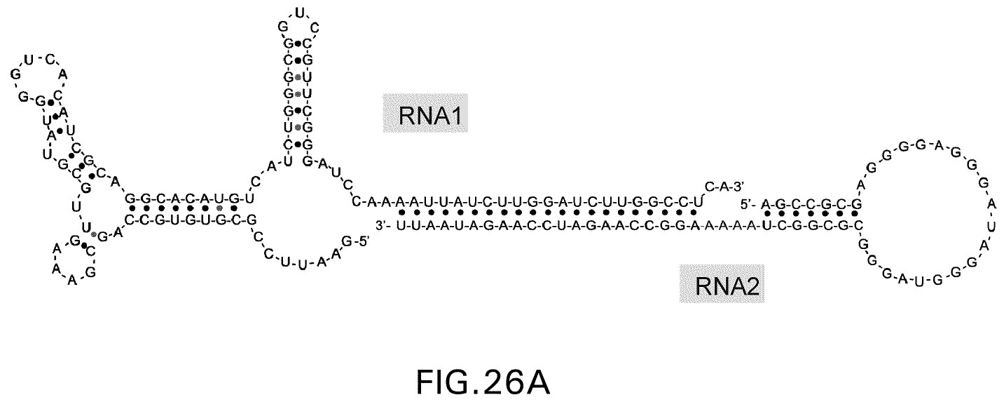

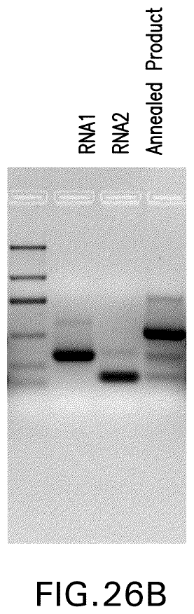

Utilizing the same synthesis method above, RNA1 and RNA2 is synthesized, purified, mixed, and annealed. The resulting products were run on a gel for confirmation (FIG. 26)
RNA1: HER3 Aptamer-UBB antisense RNA
RNA2: HER2 Aptamer-UBB sense RNA

### Example 21: EPCAM-UBB Chimeras Construction

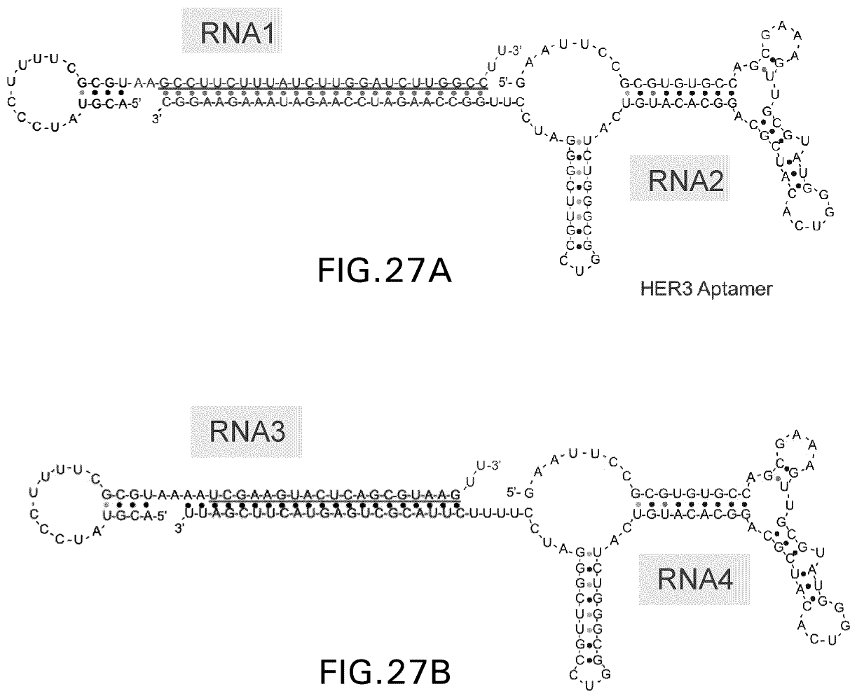

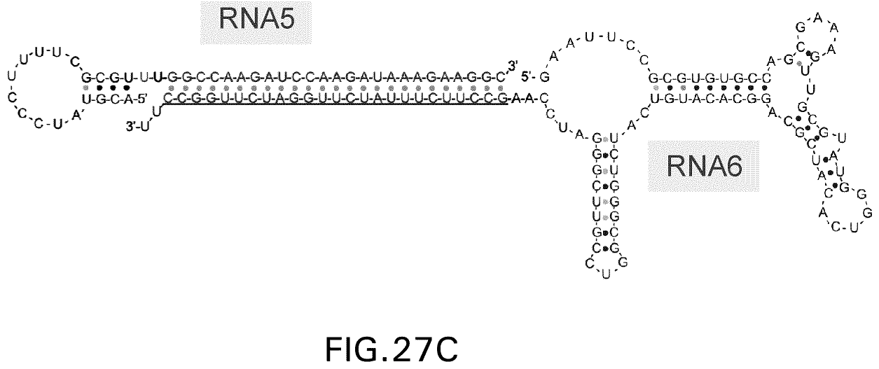

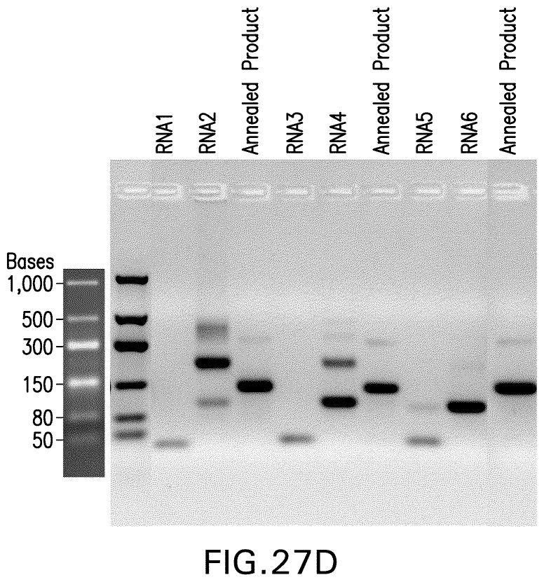

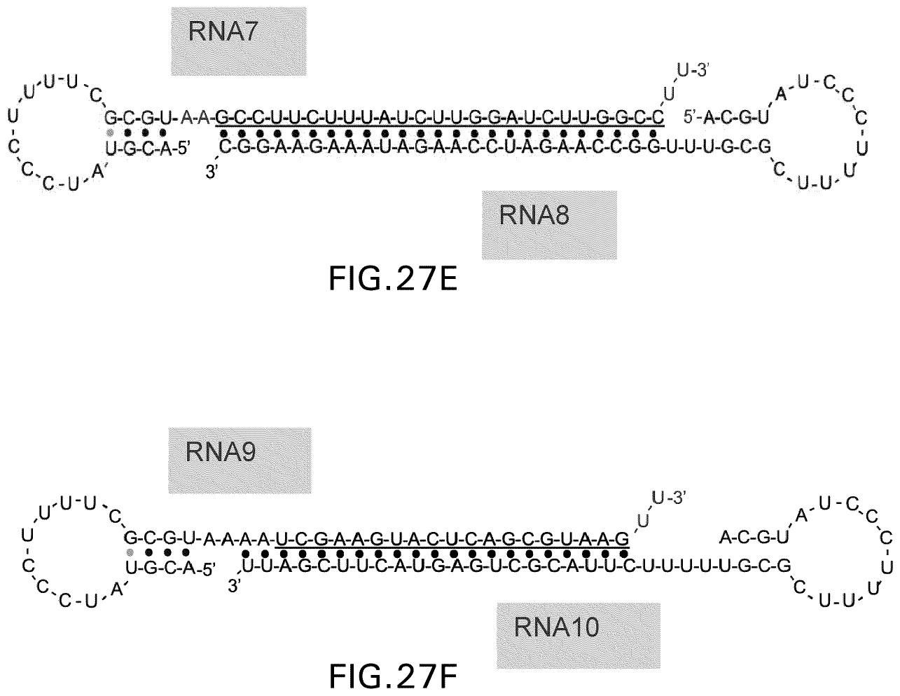

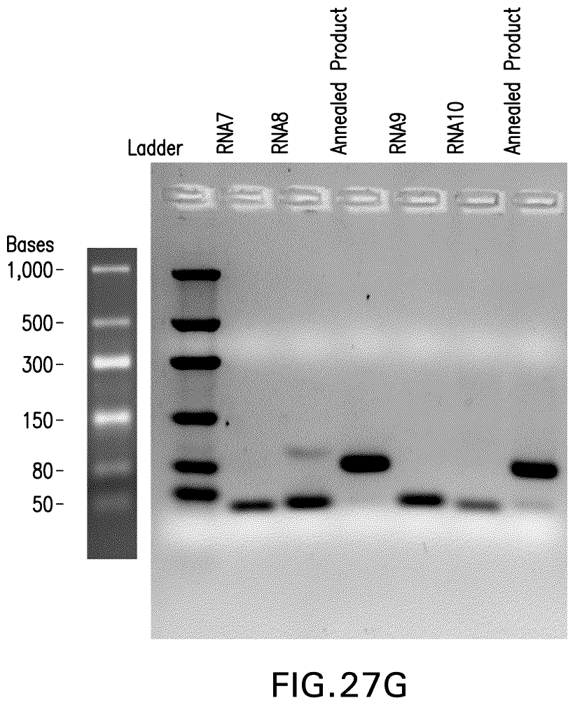

Utilizing the same synthesis method above, two RNAs are synthesized, purified, mixed, and annealed.
EPCAM-UBB-HER3 (FIG. 27A):
RNA1: EPCAM aptamer-U22ds Antisense RNA
RNA2: HER 3 Aptamer-U22ds Sense RNA
EPCAM-LUC-HER3 (FIG. 27B):
RNA3: EPCAM aptamer-Luc Antisense RNA
RNA4: HER 3 Aptamer-Luc Sense RNA
HER3-UBB-EPCAM (FIG. 27C)
RNA5: EPCAM aptamer-U22ds Sense RNA
RNA6: HER 3 Aptamer-U22ds anti-sense RNA
EPCAM-U22ds (UBB)-EPCAM (FIG. 27E)
RNA7: EPCAM aptamer with anti-sense U22ds siRNA
RNA8: EPCAM aptamer with sense U22ds siRNA
EPCAM-Luc-EPCAM (FIG. 27F)
RNA7: EPCAM aptamer with anti-sense Luc siRNA
RNA8: EPCAM aptamer with sense Luc siRNA

Alternative EPCAM aptamer sequences to be used in this construct or in other constructs of this application include:

2_pyridyl modified:
(SEQ ID NO: 202):
5′-GGGAGACAAACAAAGAGCGACAAGGGCAGAUAGCUU
UUAGUUGUGCAAUGCUCUGCACCGUCGAGUUCCCACCCAG
AAGAAGCCAGAAG-3′
(SEQ ID NO: 203):
5′-GGGAGACAAACAAAGAGCGACAAGGGCAGUGAGAUA
GUAGACGAGGAGGUUCCAUUAGAAUGCAAAUAUCACCCAG
AAGAAGCCAGAAG-3′
Benzyl Modified:
(SEQ ID NO: 204):
5′-GGGAGACAAACAAAGAGCGACAAGGGCAGAGGUACC
AAGCAGAGGGUCUAAGGGUAGCCCGGACGAGUCACCCAGA
AGAAGCCAGAAG-3′
(SEQ ID NO: 205):
5′-GGGAGACAAACAAAGAGCGACAAGGGCAGCAUCUGCU
AGUAAUGUUCGGCGGUCGAACUCUACUUGGAACACCCAG
AAGAAGCCAGAAG-3′
(SEQ ID NO: 206:
5′-GGGAGACAAACAAAGAGCGACAAGGGCAGAGACAGGU
UAGGGGAAAGUGUGUUAAACUUUAAAGUAAUUCACCCAGA
AGAAGCCAGAAG-3′

Alternative HER2 aptamer sequences to be used in this construct or in other constructs of this application include:

2_pyridyl modified:
(SEQ ID NO: 207):
5′-GGGAGACAAACAAAGAGCGACAAGGGCAGGUAGGACGUCAGU
UUAACGCAAGCGCGUUACACCUAGAUCCACCCAGAAGAAGCCAGA
AG-3′
(SEQ ID NO: 208):
5′-GGGAGACAAACAAAGAGCGACAAGGGCAGCCUAUUUUGGGGC
UGUGACAUAUUGUCAAAUGCUAAACGGCACCCAGAAGAAGCCAGA
AG-3′
(SEQ ID NO: 209):
5′-GGGAGACAAACAAAGAGCGACAAGGGCAGAGCAGUUUCGUUG
GGCGUCGGUCUAAUAGACUGACUGGGGCACCCAGAAGAAGCCAGA
AG-3′
Benzyl Modified:
(SEQ ID NO: 210):
5′-GGGAGACAAACAAAGAGCGACAAGGGCAGGUCCAAUCUGAGU
GAUGUCUGUCAAGACCUAGAGAAGUACCACCCAGAAGAAGCCAGA
AG-3′
(SEQ ID NO: 211):
5′-GGGAGACAAACAAAGAGCGACAAGGGCAGAGUGAUGUCUGUC
AAGACCGGGCUCUACCGCUGGUUCAAGCACCCAGAAGAAGCCAGA
AG-3′
(SEQ ID NO: 212):
5′-GGGAGACAAACAAAGAGCGACAAGGGCAGUGGGAGCACUAGA
AUGGUCUAUAUUAAUGUUAGCGCAGUGCACCCAGAAGAAGCCAGA
AG-3′
(SEQ ID NO: 213):
5′-AGGAGAUGCGUAGGGUGGACUGAGUGAUGUCUGUCAAGACCU
CGUCCAAAC-3′
(SEQ ID NO: 214):
5′-AGGAGUGAUGUCUGUCAAGACCGAUUGUCUGCCACUCAAUCG
GGACCAAAC-3′
(SEQ ID NO: 215):
5′-AGGAGUGAUGUCUGUCAAGACCAGGUGCUGAGGUGACUCUGU
AAUUCAAAC-3′

Resulting products were run on a gel for confirmation (FIGS. 27D and 27G).

### Example 22: Building and Testing Bispecific Aptamer-siRNA: PSMA—BIRC5-UBB/UBC-PSMA

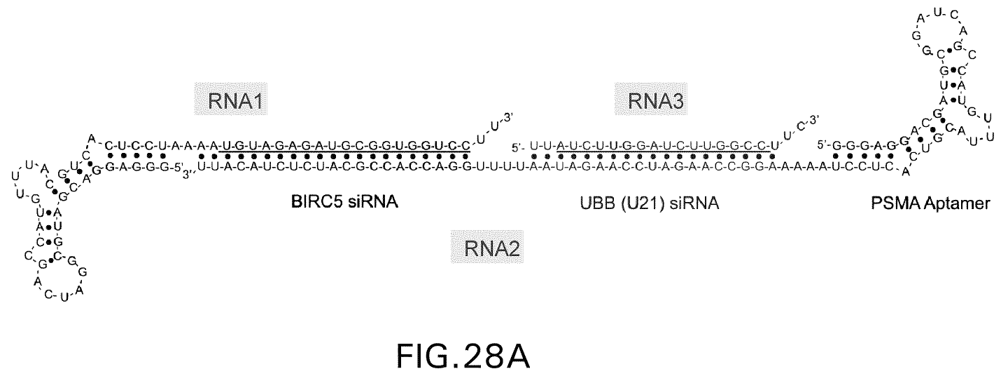

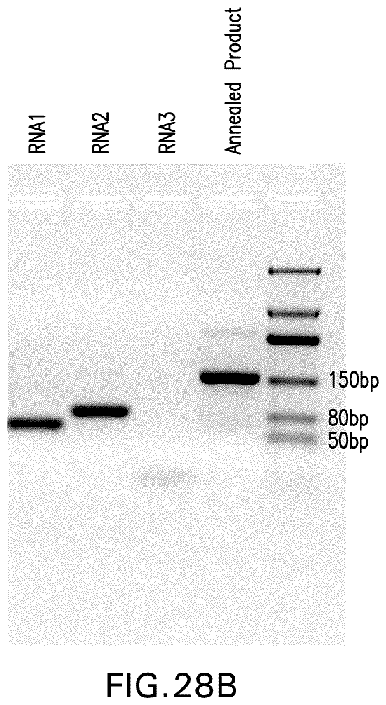

(FIG. 28A) Three RNAs are generated by in vitro transcription, with PCR products as templates.
RNA1: PSMA aptamer-BIRC5 antisense RNA
RNA2: PSMA aptamer-UBB/UBC sense siRNA and BIRC5 sense siRNA
RNA3: UBB/UBC anti-sense strand

### PSMA Aptamer Sequence:

(SEQ ID NO: 216):
5′-GGGAGGACGAUGCGGAUCAGCCAUGUUUACGUCACUCCUA-3′

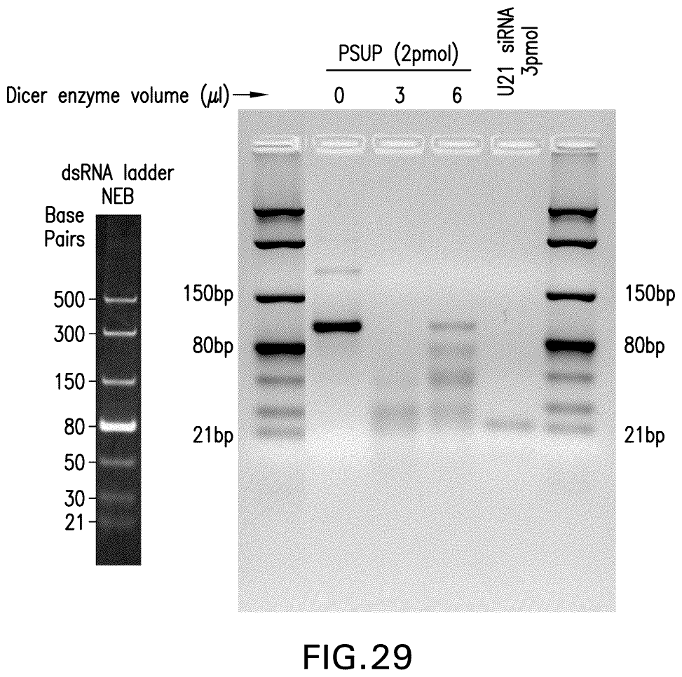

The PCR products are sequenced or put into T-A cloning pCR2.1 vector (Invitrogen) and sequenced. Transcription is performed with TranscriptAid T7 High Yield Transcription Kit following manufacture's instruction. 2′F-modified pyrimidines (TriLink, San Diego, CA) are incorporated into RNA to replace CTP and UTP. The transcribed RNAs are purified with phenol/chloroform/isoamyl alcohol (25:24:1) (Sigma-Aldrich), precipitated with isopropanol (Sigma-Aldrich) followed by cold 70% ethanol wash. The RNA pellets are dissolved in nuclease free water (IDT). The three RNAs are mixed at molar ratio 1:1:1 and annealed to form one entity by heated at 94° C. for 3 min followed by slowly cooling to room temperature within 1 h. Resulting products were run on a gel for confirmation (FIG. 28B). 2 pmol of product was treated with 0, 3, or 6 μmol of dicer enzyme for 16 hours in order to confirm that the product is able to be cleaved by the enzyme. A gel was run on the resulting product for confirmation (FIG. 29).

### Example 23: Building Bispecific Aptamer-siRNA: DHX9-UBB-DHX9

Two RNAs are generated by in vitro transcription, with PCR products as templates
RNA1: DHX9 aptamer-UBB sense RNA

(SEQ ID NO: 217):
5′-GCCCAGCAUGCAUUACUGAUCGUGGUGUUU
GCUUAGCCCAAAGGCCAAGAUCCAAGAUAAAGAAGGC-3′

RNA2: DHX9 aptamer-UBB anti-sense siRNA

(SEQ ID NO: 218):
5′-GCCCAGCAUGCAUUACUGAUCGUGGUGUUU
GCUUAGCCCAAAGCCUUCUUUAUCUUGGAUCUUGGCCUU-3′

DHX9 Aptamer Sequence:

(SEQ ID NO: 219):
5-GCCCAGCAUGCAUUACUGAUCGUGGUGUUUGCUUAGCCCA-3′

U22ds (SEQ ID NO: 156) is utilized as the UBB targeting sequence.

The PCR products are sequenced or put into T-A cloning pCR2.1 vector (Invitrogen) and sequenced. Transcription is performed with TranscriptAid T7 High Yield Transcription Kit following manufacture's instruction. 2′F-modified pyrimidines (TriLink, San Diego, CA) are incorporated into RNA to replace CTP and UTP. The transcribed RNAs are purified with phenol/chloroform/isoamyl alcohol (25:24:1) (Sigma-Aldrich), precipitated with isopropanol (Sigma-Aldrich) followed by cold 70% ethanol wash. The RNA pellets are dissolved in nuclease free water (IDT). The RNAs are mixed at molar ratio 1:1 and annealed to form one entity by heated at 94° C. for 3 min followed by slowly cooling to room temperature within 1 h. Resulting products were run on a gel for confirmation.

### Example 24 In Vitro Aptamer-siRNA Inhibition of UBB Expression

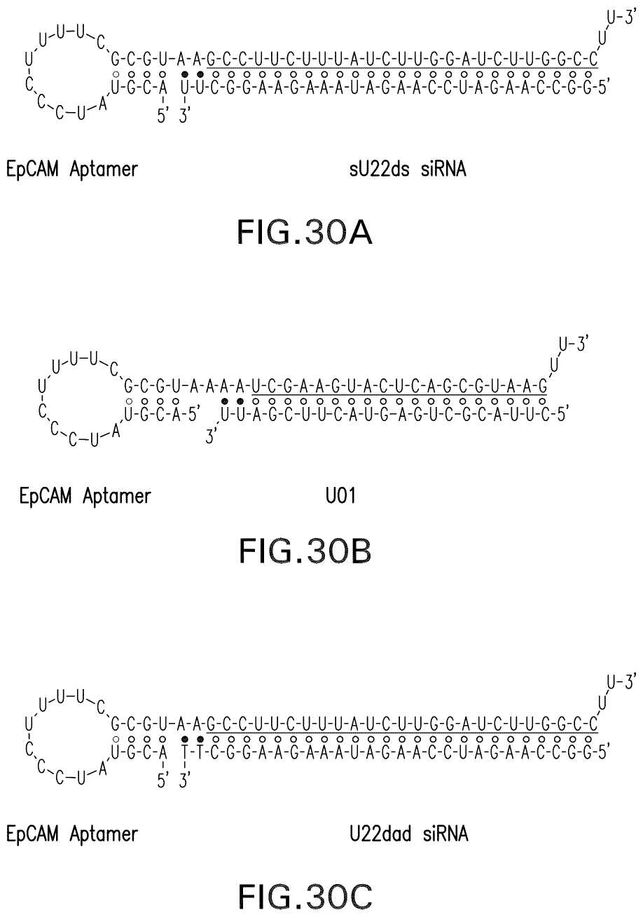

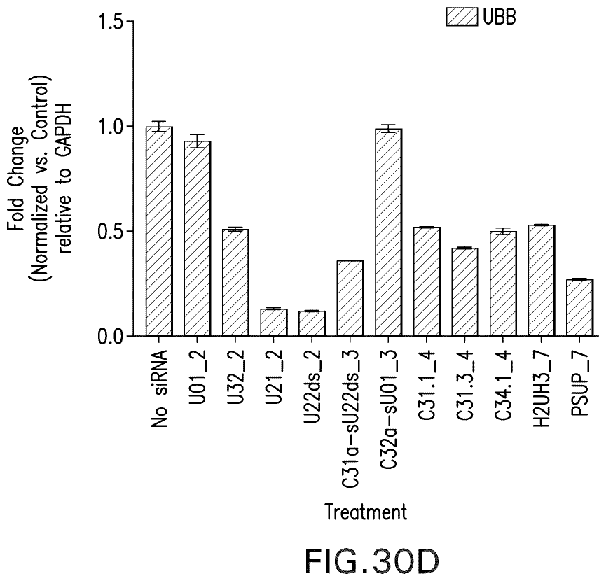

HCT-116 cells were transfected with various Aptamer-siRNA compositions with a transfection reagent ration of 6:1 for 48 hours and expression level of the target UBB was measured using qPCR. Compositions included previously disclosed controls as well as partial Aptamer-siRNA constructs shown in FIG. 30A and FIG. 30B. FIG. 30A (C31a/sU22ds) is an EpCAM aptamer conjugated to the active U22 siRNA which FIG. 30B (C32a/sU01) is the same aptamer conjugated to control. C31.1 is the construct disclosed in FIG. 27A, C31.3 is the construct disclosed in FIG. 27E, C34.1 is the construct disclosed in FIG. 27C, H2UH3 is the construct disclosed in FIG. 26A, and PSUP is the construct disclosed in FIG. 28A. Results demonstrate that active aptamer-siRNA constructs are able to inhibit UBB expression over control (FIG. 30D)

### Example 25: In Vitro Viability Studies

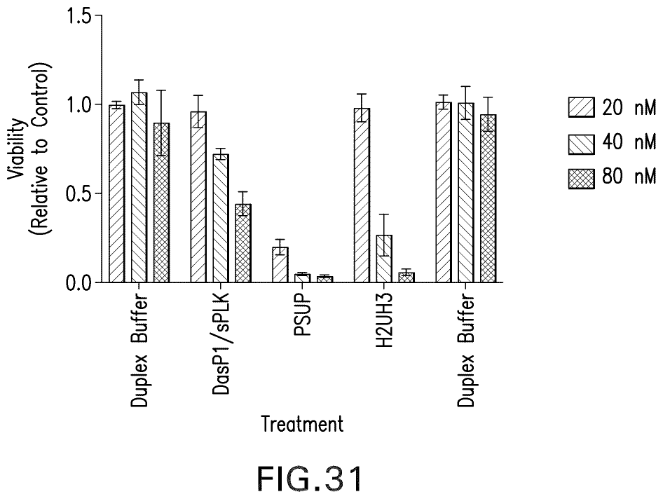

HCT116 cells were treated with previously described compositions as well as DasP1/sPLK, a PSMA aptamer-PLK1 siRNA construct. The cells treated with PSUP, PSMA aptamer-BIRC5 siRNA-UBB siRNA-PSMA aptamer, demonstrated the most significant toxicity at the lowest concentrations to colon cancer cells. H2UH3 (HER3 aptamer-U21 siRNA-HER2 aptamer) also demonstrated significant toxicity to cancer cells at a lower concentration than control (FIG. 31)

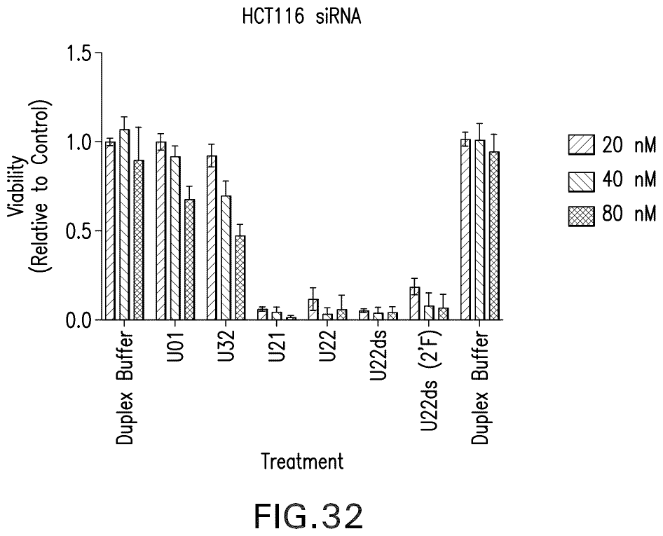

Additionally, HCT116 cells were transfected and treated for 72 hours with previously described variations of the multi-targeting UBB/UBC siRNA. Transfection reagent ratio was 6:1 and cells were treated with 20, 40, or 60 nM of RNA. Viability was measured using cell titer glow. The active siRNAs (U21, U22, U22ds, and U22ds (2′F) showed significant toxicity to the colon cancer cell compared to control (FIG. 32).

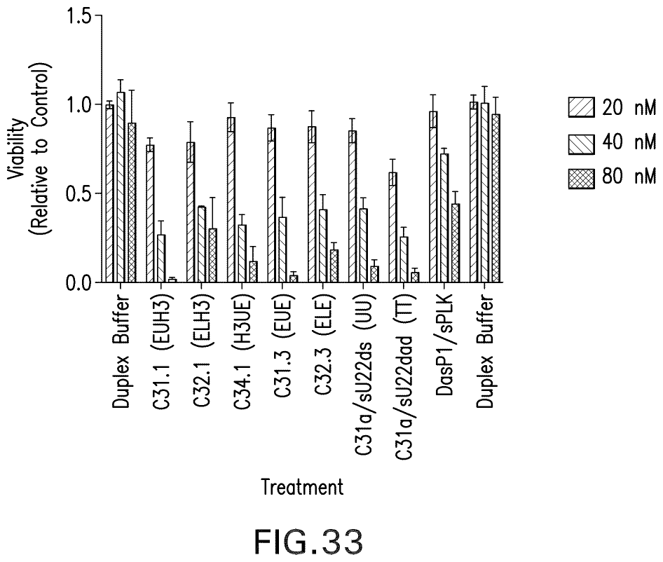

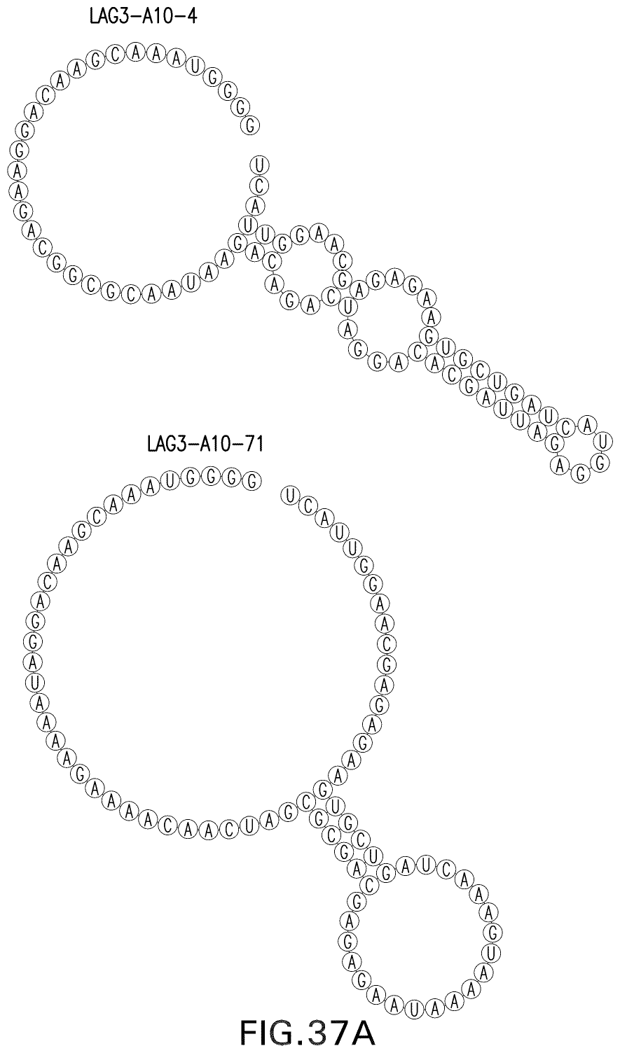

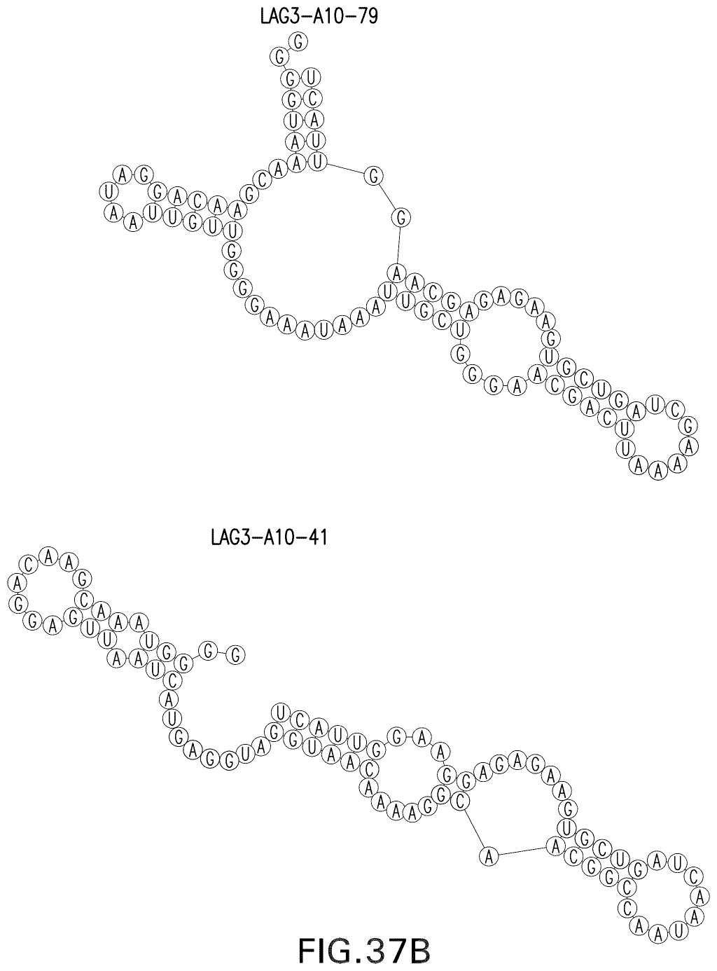

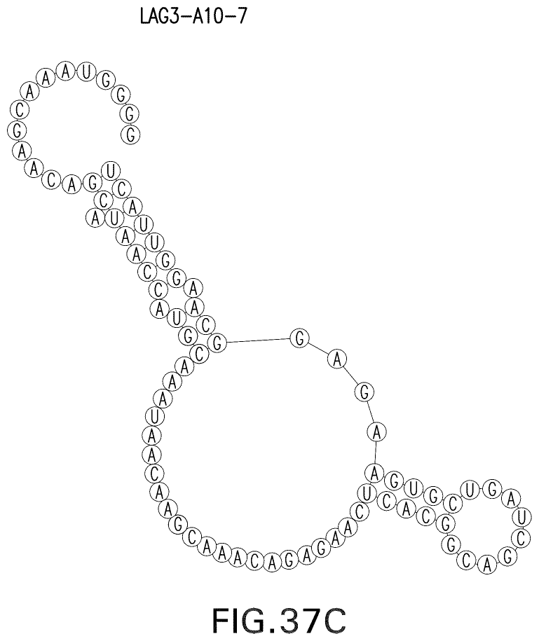

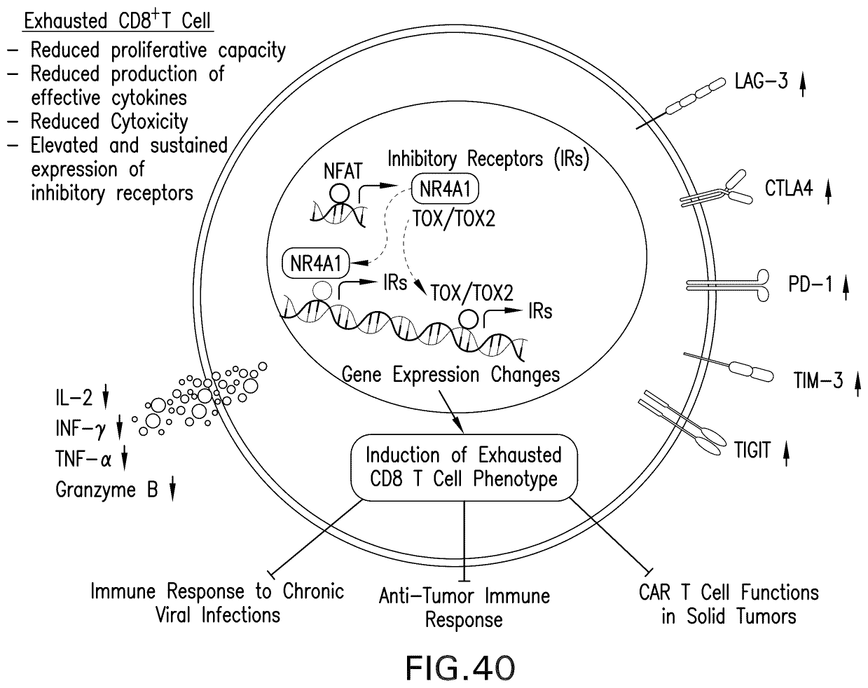

Additionally, HCT116 cells were transfected and treated for 72 hours with various aptamer-siRNA constructs, some previously described. C32.1 is the construct disclosed in FIG. 37B, C32.1 is the construct disclosed in FIG. 37F, C31a/sU22dad (TT) is the partial aptamer-siRNA construct disclosed in FIG. 40C. Transfection reagent ratio was 6:1 and cells were treated with 20, 40, or 60 nM of RNA. Viability was measured using cell titer glow. The constructs that included a dual targeting UBB/UBC siRNA demonstrated the most significant toxicity to the cancer cells compared to control at higher concentrations with 31.1 and 31.3 showing the most significant (FIG. 33).

### Example 26: PD-1-NR4A1-VHL-CTLA4 Construct

Three RNAs are generated by in vitro transcription, with PCR products as templates.
RNA1: PD-1 aptamer-NR4A1 antisense siRNA
RNA2: CTLA4 aptamer and VHL sense siRNA and NR4A1 sense siRNA
RNA3: VHL anti-sense strand

### Anti-PD1 Aptamer Sequences:

(SEQ ID NO: 220):
5′-GGCUGGGGUGCGACAUUAUGUUCGUUAAGGAUCAAUCGCUC-3′
(SEQ ID NO: 221):
5′-UUAUGAUGCAAAAACGAACUGGAAUGGCCAUGCAGGUACA-3′
(SEQ ID NO: 222):
5′-GGUUGGGGUGCGACAUUAUGUUCGUUAAGGAUCAAUCGCUC-3′
(SEQ ID NO: 223):
5′-GAUUUGGAGAGCAUUAUGUUAGGUUAAGGAUCAAUCUUCUA-3′
(SEQ ID NO: 224):
5′-GGCUGGGGUGCGACAUUAUGUUCGUUAAGGAUCAAUCGCUU-3′
SEQ ID NO: 225):
5′-GGGGUAAACGAACAGGAUUUUUUUAACGAGCUAUAUUGUUUCCUGU
UGCCCGUCCGUCUAGUCGUGAAGAGAGCAAGGUUACU-3′ (10B-1)
(SEQ ID NO: 226):
5′-GGGGUAAACGAACAGGACGACGGGUCGAAGCUGAAUAGGUAACCAA
UCACGGCAUAACUAGUCGUGAAGAGAGCAAGGUUACU-3′ (10B-10)
(SEQ ID NO: 227):
5′-GGGGUAAACGAACAGGAUGAGGGAGCAAAAAGGGCGAAAAUGCAGU
AACUAAACGUUCUAGUCGUGAAGAGAGCAAGGUUACU-3′ (10B-14)
(SEQ ID NO: 228):
5′-GGGGUAAACGAACAGGAUUUUUUUAACGAGCUAUAUUAUUUCCUGU
UGCCCGUCCGUCUAGUCGUGAAGAGAGCAAGGUUACU-3′ (10B-68)
(SEQ ID NO: 229):
5′-GGGGUAAACGAACAGGAACCAUUAAAUCAUAAGGAGAAAGAUGAUG
UGCGCGACAUAACUAGUCGUGAAGAGAGCAAGGUUACU-3′ (10B-84)
2_pyridyl modification:
(SEQ ID NO: 230):
5′-GGGAGACAAACAAAGAGCGACAAGGGCAGCGGGUUAUCACGUUGGG
AACGGGCCAUCAACUCUUCUCACCACCCAGAAGAAGCCAGAAG-3′
(SEQ ID NO: 231):
5′-GGGAGACAAACAAAGAGCGACAAGGGCAGUAGUGAGGGAUUCACCA
GAGUGAAUGCGCUCCUCGGAAAUCACCCAGAAGAAGCCAGAAG-3′
(SEQ ID NO: 232):
5′-GGGAGACAAACAAAGAGCGACAAGGGCAGAACGGGCAAUGUCCAAG
GUGAGGCAGUUUGUAUGGACACACACCCAGAAGAAGCCAGAAG-3′
Benzyl Modification:
(SEQ ID NO: 233):
5′-GGGAGACAAACAAAGAGCGACAAGGGCAGUUGAGAUUGAGGAGUCA
GACCUGCGUCUCUAGUAACAAUGCACCCAGAAGAAGCCAGAAG-3′
(SEQ ID NO: 234):
5′-GGGAGACAAACAAAGAGCGACAAGGGCAGAGUGGACGGUCGGCUAG
AGCCGGGAGGAAUUCCUUGUGACCACCCAGAAGAAGCCAGAAG-3′
(SEQ ID NO: 235):
5′-GGGAGACAAACAAAGAGCGACAAGGGCAGUUUGACAAUGUACCUUU
AAUUACGGAUUGUACCUUGGGCGCACCCAGAAGAAGCCAGAAG-3′

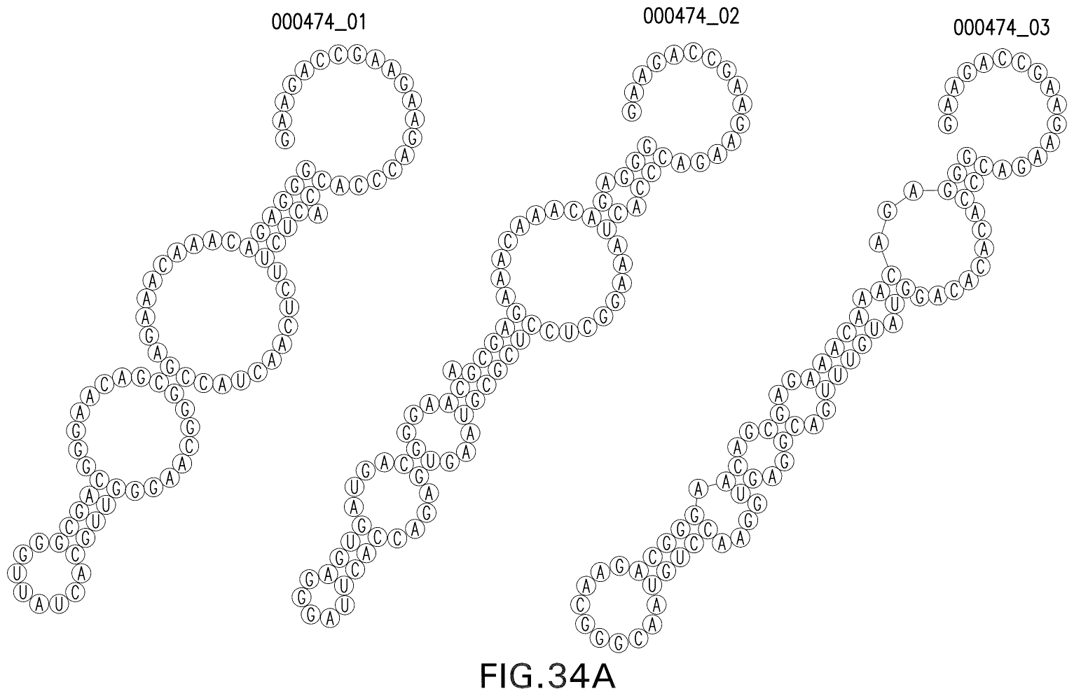

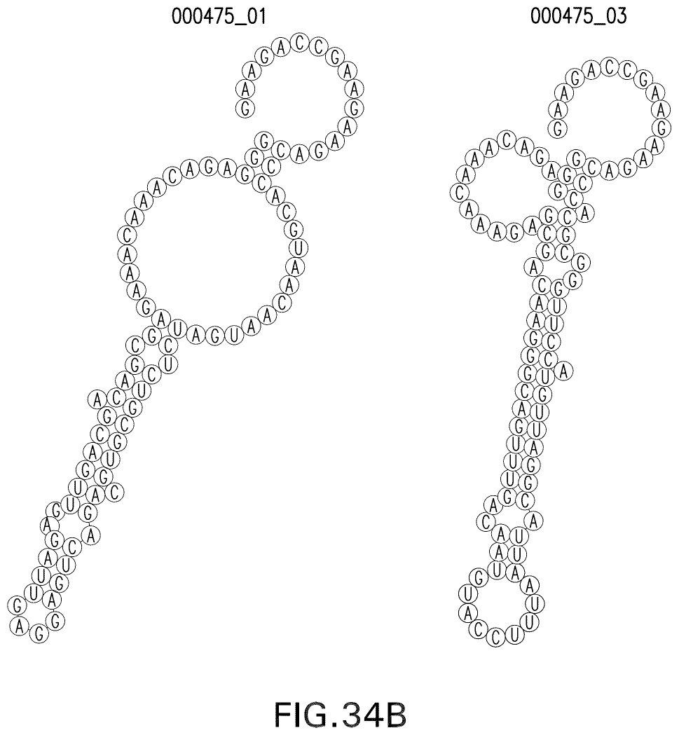

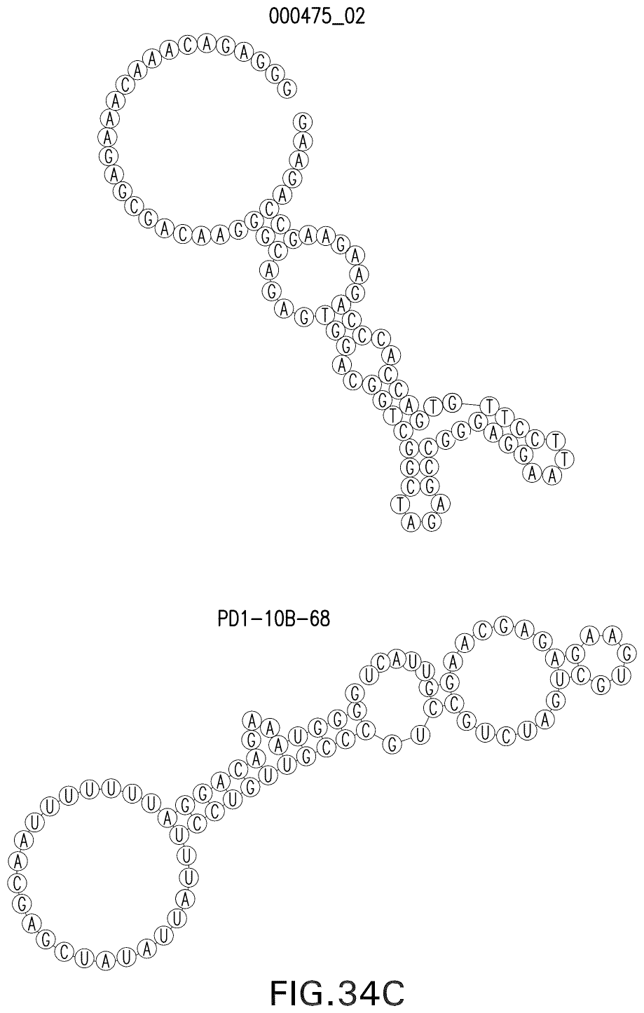

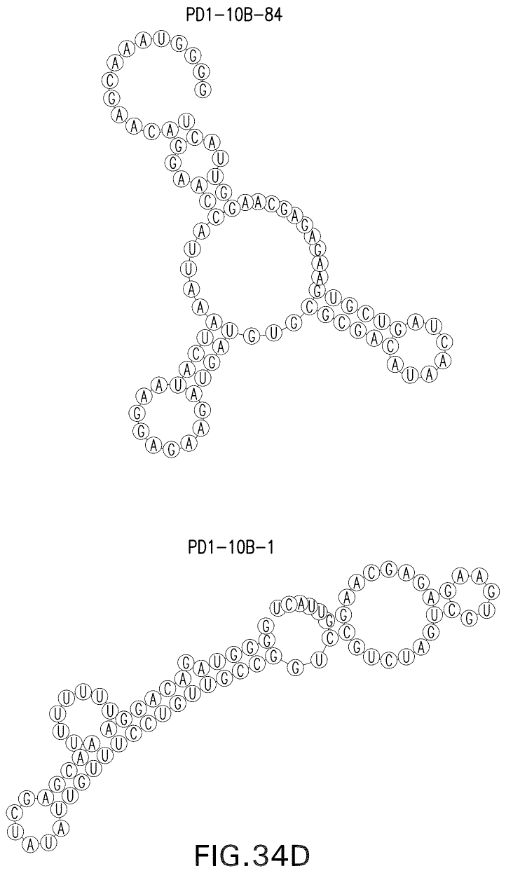

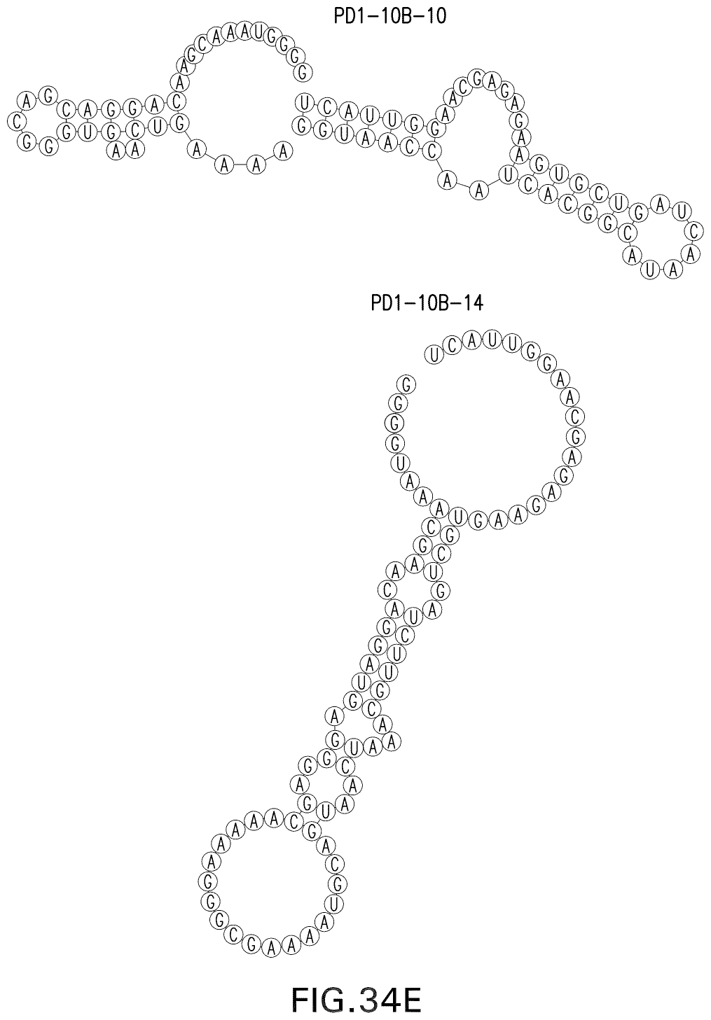

Binding structures of select aptamers are shown in FIG. 34.

### CTLA-4 Aptamer Sequences:

(SEQ ID NO: 236):
5′-GGGAGAGAGGAAGAGGGAUGGGCCGACGUGCCGCA-3′
2_pyridyl modification:
(SEQ ID NO: 237):
5′-GGGAGACAAACAAAGAGCGACAAGGGCAGAAUUACAAUAGCUAUAG
UCCGGGCACCAUGCUUGUAAAUCCACCCAGAAGAAGCCAGAAG-3′
(SEQ ID NO: 238):
5′-GGGAGACAAACAAAGAGCGACAAGGGCAGGUAGGACGCUAGCAGAC
UAGAAUGUAUCUAUGCUUAGAUCCACCCAGAAGAAGCCAGAAG-3′
(SEQ ID NO: 239):
5′-GGGAGACAAACAAAGAGCGACAAGGGCAGGCUAGUAUUACAAUGUC
GUGGAAAAGCCGUGCGGGGUAUCCACCCAGAAGAAGCCAGAAG-3′
Benzyl Modification:
(SEQ ID NO: 240):
5′-GGGAGACAAACAAAGAGCGACAAGGGCAGGGAGCCAUUCUUGAAAU
UGUCAGUUUGAUUGUGCUCAGGUCACCCAGAAGAAGCCAGAAG-3′
(SEQ ID NO: 241):
5′-GGGAGACAAACAAAGAGCGACAAGGGCAGAAAGUACAAUGGUUGAC
AUAUACCGUCGGUUUAUCCUAUGCACCCAGAAGAAGCCAGAAG-3′
(SEQ ID NO: 242):
5′-GGGAGACAAACAAAGAGCGACAAGGGCAGGUAGGCUAUCGCUGCUU
GAUCGUCUGAUCAGAGCCUAUACCACCCAGAAGAAGCCAGAAG-3′
(SEQ ID NO: 243):
5′-GGGGUAAACGAACAGGAAACAGAUGGCCAACACAGGCGAAGCAUAG
ACUAGGAACGGCUAGUCGUGAAGAGAGCAAGGUUACU-3′ (CTLA4-
A10-6)
(SEQ ID NO: 244):
5′-GGGGUAAACGAACAGGACUUGAUGUGAAAAGGCGACGCGAUGAGAC
GAAGGGCUUCUAGUCGUGAAGAGAGCAAGGUUACU-3′ (CTLA4-
A10-38)
(SEQ ID NO: 245):
5′-GGGGUAAACGAACAGGAAGUAGACUAGACGGCGGCGAUAACCAGAU
AACGACAUUCUCUAGUCGUGAAGAGAGCAAGGUUACU-3′ (CTLA4-
A10-14)
(SEQ ID NO: 246):
5′-GGGGUAAACGAACAGGACCGAGUGAGACGGGUAGUGGACAAAUGAA
GUAGUGUGGUCCUAGUCGUGAAGAGAGCAAGGUUACU-3′ (CTLA4-
A10-2)
(SEQ ID NO: 247):
5′-GGGGUAAACGAACAGGACUUUUAAUUUCACGCCGCACGAUCCGGAA
AAACGACUUGACUAGUCGUGAAGAGAGCAAGGUUACU-3′ (CTLA4-
A10-13)

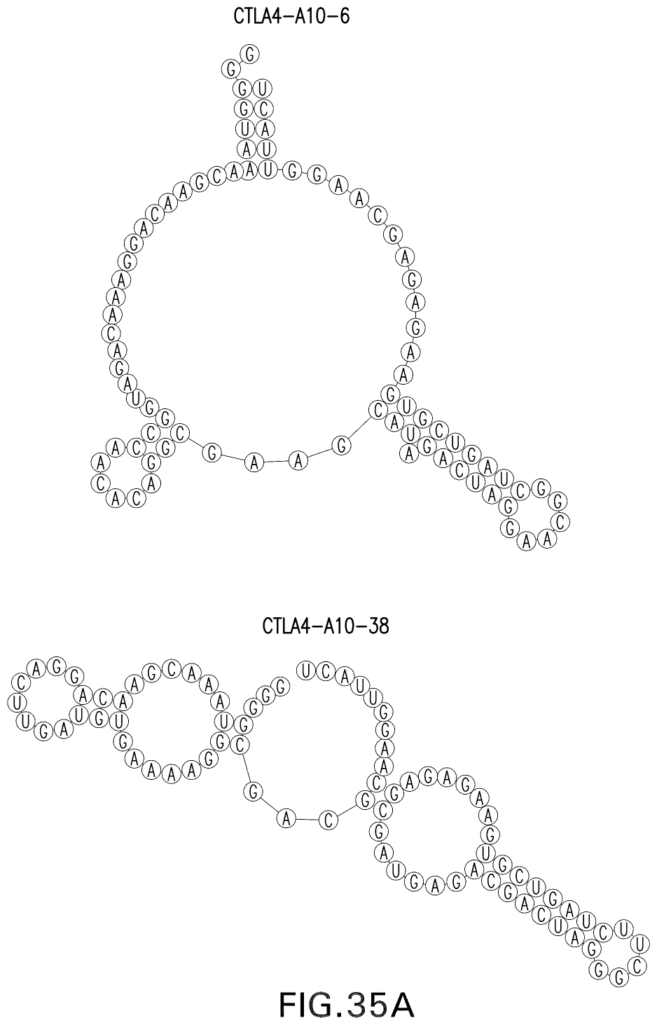

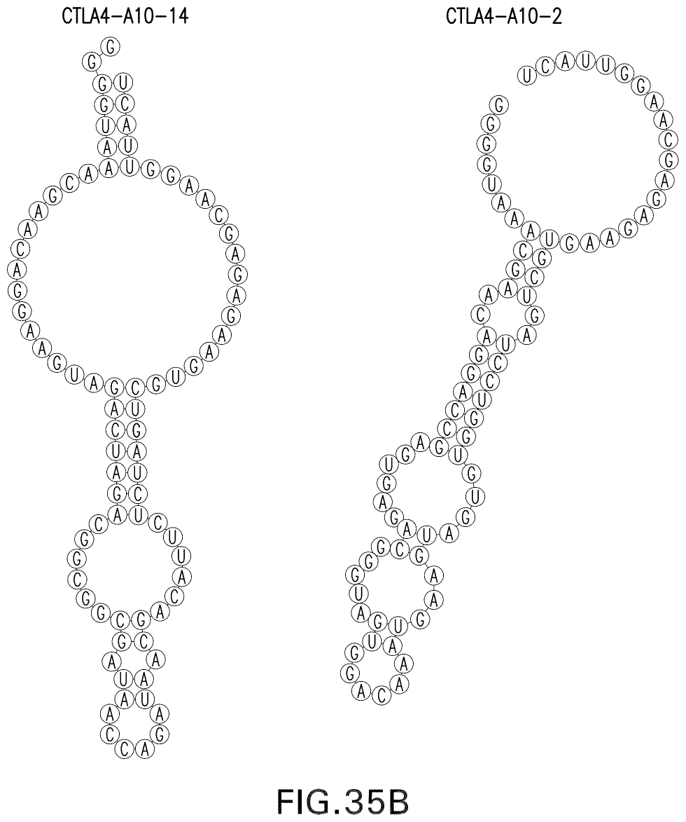

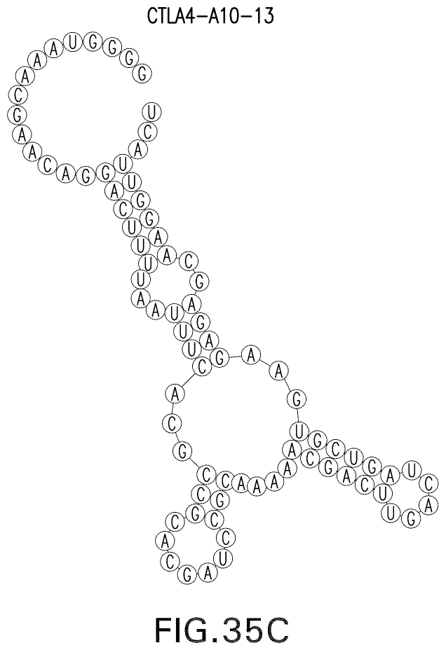

Binding structures of select aptamers are shown in FIG. 35.

### Target Sequences of NR4A1:

(SEQ ID NO: 248):
5′-CTGATTAATATATTTAATATA-3′
(SEQ ID NO: 249):
5′-CTCCTTCCACATGTACATAAA-3′
(SEQ ID NO: 250):
5′-CAGCATTATGGTGTCCGCACA-3′
(SEQ ID NO: 251):
5′-CAGCACCTTCATGGACGGCTA-3′
(SEQ ID NO: 252):
5′-GCACCTTCATGGACGGCTA-3′ (hNR4A1.1E2)
(SEQ ID NO: 253):
5′-GCATATGGTGTCCGCACA-3′ (hNR4A1.2E2)
(SEQ ID NO: 254):
5′-TGAAGGAAGTGTCCGAAC-3′ (hNR4A1.3E2)
(SEQ ID NO: 255):
5′-CTGCAGAACCGCATCGCCA-3′ (hNR4A1.4E2)
(SEQ ID NO: 256):
5′-TGCTGTGTGTGGGGACAAC-3′
(SEQ ID NO: 257):
5′-GGGCTGCAAGGGCTTCTTC-3′
(SEQ ID NO: 258):
5′-GCGCACAGTGCAGAAAAAC-3′
(SEQ ID NO: 259):
5′-CAGTGGCTCTGACTACT-3′
(SEQ ID NO: 260):
5′-CCACTTCTCCACACCTTGA-3′
(SEQ ID NO: 261):
5′-GGCTTGAGCTGCAGAATG-3′
(SEQ ID NO: 262):
5′-CACAGCTTGCTTGTCGATGTC-3′
(SEQ ID NO: 263):
5′-GGTCCCTGCACAGCTTGCTTGTCGA-3′
(SEQ ID NO: 264):
5′-CCGGTTCTCTGGAGGTCATCCGCAA-3′
(SEQ ID NO: 265):
5′-CAGCATTATGGTGTCCGCACATGTG-3′

### Target Sequences of VHL:

(SEQ ID NO: 266):
5′-AATGTTGACGGACAGCCTATT-3′
(SEQ ID NO: 267):
5′-AAGAGTACGGCCCTGAAGAAG-3′
(SEQ ID NO: 268):
5′-AAGGAGGTTTGTATAAGTAAT-3′
(SEQ ID NO: 269):
5′-CAGGAGCGCATTGCACATCAA-3′
(SEQ ID NO: 270):
5′-CCCTATTAGATACACTTCTTA-3′
(SEQ ID NO: 271):
5′-TAAGGAGGTTTGTATAAGTAA-3′
(SEQ ID NO: 272):
5′-CCTAGTCAAGCCTGAGAATTA-3′
(SEQ ID NO: 273):
5′-CTGCCAGTGTATACTCTGA-3′
(SEQ ID NO: 274):
5′-ATACACTCGGTAGCTGTGG-3′

The PCR products are processed according to the methods previously stated.

### Example 27: Building Bispecific Aptamer-siRNA: TROP-2-UBB/UBC-HER3

Two RNAs are generated by in vitro transcription, with PCR products as templates.
RNA1: TROP2 aptamer-UBB/UBC antisense RNA
RNA2: HER3 aptamer and UBB/UBC sense siRNA.

The PCR products are processed as previously described.

### Trop 2 Aptamer:

(SEQ ID NO: 275):
5′-UAUACAUUCUUGGUUCAUAAAGGAUAAGGCCUAAGUCGGGU-3′
Benzyl modification:
(SEQ ID NO: 276):
5′-AGGAGUAUACAUUCUUGGUUCAUAAAGGAUAAGGCCUAAGUCGGGU
CAAAC-3′
(SEQ ID NO: 277):
5′-GGGAGACAAGAAUAAACGCUCAAGACACGGAUACAUAAUGCUGUCU
UGAUUUACAAACUGAGCUUCGACAGGAGGCUCACAACAGGC-3′ (S10)
(SEQ ID NO: 278)
5′-GGGAGACAAGAAUAAACGCUCAAUGAGCUUACAGCGGCCAUUGAUU
UACUAACGGACUGAGCAUUCGACAGGAGGCUCACAACAGGC-3′ (S09)

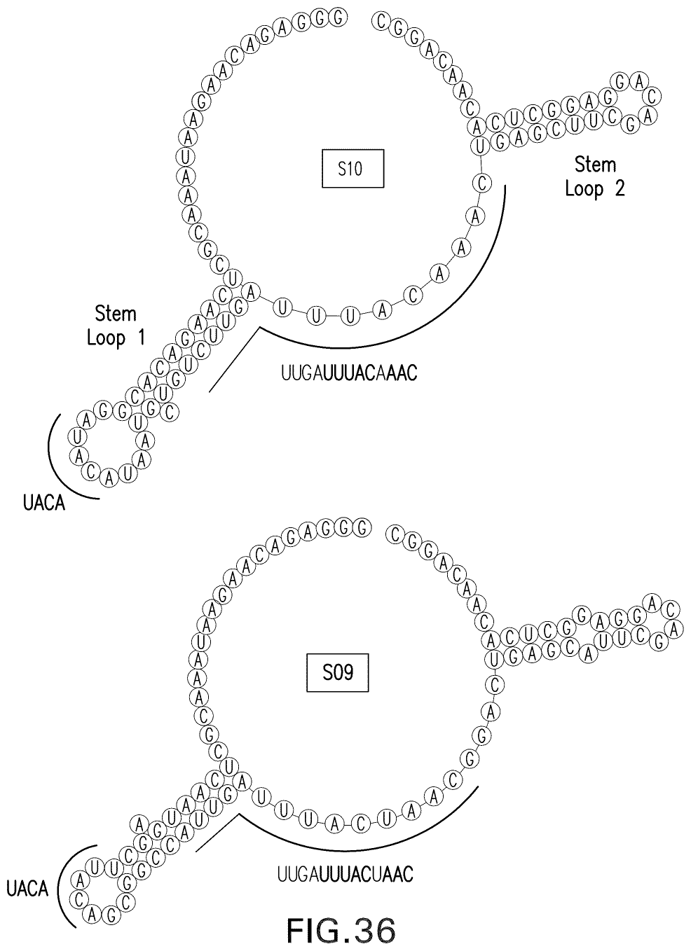

See FIG. 36 for example binding structures.

### HER3 Aptamer:

(SEQ ID NO: 279):
5′-GAAUUCCGCGUGUGCCAGCGAAAGUUGCGUAUGGGUCACAUCGCAG
GCACAUGUCAUCUGGGGGGUCCGUUCGGGAUCC-3′
Benzyl Modified: (SEQ ID NO: 280):
5′-AGGAGGGUUGCGUUGCAAGUAACAGAAAGGAAUUUGAAAAUUGUGG
CAAAC-3′
4-Pyridyl Modified: (SEQ ID NO: 281):
5′-AGGAGGUUGGCAAUCCCGGAUUGAGGAAUCGCAUGACGCUAUUAAC
CAAAC-3′

### Example 28: Building Bispecific Aptamer-siRNA: LAG-3-NR4A1-PD-1

Two RNAs are generated by in vitro transcription, with PCR products as templates.
RNA1: LAG-3 aptamer-NR4A1 antisense RNA
RNA2: PD-1 aptamer and NR4A1 sense siRNA.

The PCR products are processed as previously described.

### Lag-3 Aptamer Sequence:

(SEQ ID NO: 282):
5′-GGGAGAGAGAUAUAAGGGCCUCCUGAUACCCGCUGCUAUCUGGACC
GAUCCCAUUACCAAAUUCUCUCCC-3′
(SEQ ID NO: 283):
5′-GGGGUAAACGAACAGGAAGACGGCGCAAUAAGACAGACUAGGACAC
GAUUAGAGGUACUAGUCGUGAAGAGAGCAAGGUUACU-3′ (LAG3-
A10-4)
(SEQ ID NO: 284:
5′-GGGGUAAACGAACAGGAUAAAAGAAAACAACUAGCGCGACGAGAGA
AUAAAAUGAAACUAGUCGUGAAGAGAGCAAGGUUACU (LAG3-A10-
71)
(SEQ ID NO: 285):
5′-GGGGUAAACGAACAGGAUAAUUGUUGGGGAAAUAAAUUGCUGGGAA
CGACUUAAAAGCUAGUCGUGAAGAGAGCAAGGUUACU-3′ (LAG3-
A10-79)
(SEQ ID NO: 286):
5′-GGGGUAAACGAACAGGAGUUAAUCAUGAGGUAGGUAACAAAAGGCA
ACGGCCAAUAACUAGUCGUGAAGAGAGCAAGGUUACU (LAG3-A10-
41)
(SEQ ID NO: 287):
5′-GGGGUAAACGAACAGGAUAACCAUGCAAAUAACAAGCAAACAGAGA
ACUCACGCCAGCUAGUCGUGAAGAGAGCAAGGUUACU-3′ (LAG3-
A10-7)

See FIG. 37 for example binding structures.

### Example 29: Building Bispecific Aptamer-siRNA: CD73-UBB/UBC-TROP2

Two RNAs are generated by in vitro transcription, with PCR products as templates.
RNA1: CD73 aptamer-UBB/UBC antisense RNA
RNA2: TROP2 aptamer-UBB/UBC sense siRNA.

The PCR products are processed as previously described.

### CD73 Aptamer Sequence:

(SEQ ID NO: 288):
5′-UAGUAAAUGAGAGAUGAAAUCUGUAUGCGCCGCACUGAUUG-3′
Benzyl Modified:
(SEQ ID NO: 289):
5′-AGGAGUAGUGCAGCAUUGACUGAAUGUCAUACGGCAUAAGCAUCUA
CAAAC-3′
(SEQ ID NO: 290):
5′-AGGAGGACAUCGGAAACGCUGAUCUUAAUAGUGAAUUAACAUGCGA
CAAAC-3′
4-Pyridyl modified:
(SEQ ID NO: 291):
5′-AGGAGUAGUAAAUGAGAGAUGAAAUCUGUAUGCGCCGCACUGAUUG
CAAAC-3′

### Example 30: PD-1-NR4A1-VHL-PD-1 Construct

Three RNAs are generated by in vitro transcription, with PCR products as templates.
RNA1: PD-1 aptamer-NR4A1 antisense siRNA
RNA2: PD-1 aptamer and VHL sense siRNA and NR4A1 sense siRNA
RNA3: VHL anti-sense strand

PCR products are processed as previously discussed.

### Example 31: PSCA-MSI2-UBB-CD44

RNA1: PSCA aptamer-MSI2 antisense siRNA
RNA2: CD44 aptamer and UBB sense siRNA and MSI2 sense siRNA
RNA3: UBB anti-sense strand

PCR products are processed as previously discussed using sequences presented in this application.

### PSCA Aptamer Sequence:

2_pyridyl modified:
(SEQ ID NO: 292):
5′-GGGAGACAAACAAAGAGCGACAAGGGCAGGGAUGACCGGUGUAUUA
AGGUCUAACUUAACUCGGUGAUACACCCAGAAGAAGCCAGAAG-3′
(SEQ ID NO: 293):
5′-GGGAGACAAACAAAGAGCGACAAGGGCAGCUCAAAAAGGGUAGUGU
GUGGUAUAGUCUAAUCGUACCCACACCCAGAAGAAGCCAGAAG-3′
(SEQ ID NO: 294):
5′-GGGAGACAAACAAAGAGCGACAAGGGCAGUAGUGUGGUAUUGUGUA
AUAAUACCCUACUGAGGUCAAAACACCCAGAAGAAGCCAGAAG-3′
(SEQ ID NO: 295):
5′-GGGAGACAAACAAAGAGCGACAAGGGCAGCGGGUUGUCAAGAUGGG
AACGGGCCCGGAUCUUUAGCGCACACCCAGAAGAAGCCAGAAG-3′
(SEQ ID NO: 296):
5′-GGGAGACAAACAAAGAGCGACAAGGGCAGUGGGCUGUGCGCGCGAU
GAGAUCACGUUAGCGUAAUUGUGCACCCAGAAGAAGCCAGAAG-3′
(SEQ ID NO: 297):
5′-GGGAGACAAACAAAGAGCGACAAGGGCAGACAGGUCCAUCAGGCAG
AACCGAGGGAGAGUGCGCGUCGUCACCCAGAAGAAGCCAGAAG-3′
Benzyl Modified:
(SEQ ID NO: 298):
5′-GGGAGACAAACAAAGAGCGACAAGGGCAGAAGUUUGGAUUUCAAGA
UGCUCAUCACGCUCAAACUUUCACACCCAGAAGAAGCCAGAAG-3′
(SEQ ID NO: 299):
5′-GGGAGACAAACAAAGAGCGACAAGGGCAGGACGCUGCGAAAAGUGC
GAAGUUUGCAUCCUGGCCUAGUUCACCCAGAAGAAGCCAGAAG-3′
(SEQ ID NO: 300):
5′-GGGAGACAAACAAAGAGCGACAAGGGCAGUUCUCUCCACAAAGUUU
AGAUUUCAAGCGUGAGCAGGGAUCACCCAGAAGAAGCCAGAAG-3′
(SEQ ID NO: 301):
5′-GGGAGACAAACAAAGAGCGACAAGGGCAGGUGUUGAGAUGAAGGAG
UUCUAGCCCUUCGAAUGGUGUGACACCCAGAAGAAGCCAGAAG-3′
(SEQ ID NO: 302):
5′-GGGAGACAAACAAAGAGCGACAAGGGCAGAGUUGGGUAGGUUGUGA
CAGGAAUGUGAUUGGUAAGAUACCACCCAGAAGAAGCCAGAAG-3′
(SEQ ID NO: 303):
5′-GGGAGACAAACAAAGAGCGACAAGGGCAGUACUUGAGUCAUUGUAU
AGAUCUAAUUCGCGCAGAAUUGACACCCAGAAGAAGCCAGAAG-3′
(SEQ ID NO: 304):
5′-GGGAGACAAACAAAGAGCGACAAGGGCAGCCCUUGUCGCUCUUUGU
UUGUCUCCCUAUAGUGAGUCGUAUUACACCCAGAAGAAGCCAGAAG-3′

### Example 32: CD44-PIKFYVE-MAP2K1-CD133

RNA1: CD44 aptamer-PIKFYVE antisense siRNA
RNA2: CD133 aptamer and MAP2K1 sense siRNA and PIKFYVE sense siRNA
RNA3: MAP2K1 anti-sense strand

PCR products are processed as previously discussed using sequences presented in this application.

### Example 33: PSMA-UBB/UBC-PSMA

RNA1: PSMA aptamer-UBB/UBC antisense RNA
RNA2: PSMA aptamer and UBB/UBC sense siRNA.

### CD44 Aptamer Sequence:

2_pyridyl modified:
(SEQ ID NO: 305):
5′-GGGAGACAAACAAAGAGCGACAAGGGCAGGUAGGACGCUAGCAGAC
UAGAAUGAAUCUAUGCUUAGAUCCACCCAGAAGAAGCCAGAAG-3′
Benzyl Modified:
(SEQ ID NO: 306):
5′-GGGAGACAAACAAAGAGCGACAAGGGCAGCGGUUAAAAUAUAGUUC
UAAGUUAGUCUGGUGAAUCCACUCACCCAGAAGAAGCCAGAAG-3′
(SEQ ID NO: 307):
5′-GGGAGACAAACAAAGAGCGACAAGGGCAGUCUGGGGGAUGGCUGUG
AGGUCCCAAUGUAUCGCAUCUCACACCCAGAAGAAGCCAGAAG-3′
(SEQ ID NO: 308):
5′-GGGAGACAAACAAAGAGCGACAAGGGCAGUUAAAAUAUAGUUCUAA
GUUAGUCUGGUGAAUCCACUCACCCAGAAGAAGCCAGAAG-3′

### Example 34: PD-1-CBLB-ADORA1/2-TIM3

RNA1: PD1 aptamer-CBLB antisense siRNA
RNA2: TIM3 aptamer and ADORA1/2 sense siRNA and CBLB sense siRNA
RNA3: ADORA1/2 anti-sense strand

PCR products are processed as previously discussed using sequences presented in this application.

### TIM3 Aptamer Sequence:

(SEQ ID NO: 309):
5′-GGGGUAAACGAACAGGAAGGGAGUCGAUUUGAGUUGUAAUUUGACC
UAUGUUAUAAUCUAGUCGUGAAGAGAGCAAGGUUACU-3′ (TIM3-
A-4)
(SEQ ID NO: 310):
5′-GGGGUAAACGAACAGGAAUGGCUACAGUAUCGAUGCAGUUUUCGAA
UGAAGUAGAAACUAGUCGUGAAGAGAGCAAGGUUACU-3′ (TIM3-
A-8)
(SEQ ID NO: 311):
5′-GGGGUAAACGAACAGGACAGGACAGCAAGCAGUAGAAAACAAGCCA
CGAAGGGGACUCUAGUCGUGAAGAGAGCAAGGUUACU-3′ (TIM3-
A-25)
(SEQ ID NO: 312):
5′-GGGGUAAACGAACAGGAUUUUGGACUGUCUAGCCGAUGUACUUAAG
UUUAUCAUUUUCUAGUCGUGAAGAGAGCAAGGUUACU-3′ (TIM3-
A-43)
(SEQ ID NO: 313):
5′-GGGGUAAACGAACAGGAGCAGUCGCUGGCUUCAUUUUUUUUUUUUU
UUUGUGCUCAACUAGUCGUGAAGAGAGCAAGGUUACU-3′ (TIM3-
A-57)

### PSMA Aptamer Sequence:

Benzyl Modified:
(SEQ ID NO: 314):
5′-AGGAGACACAUGUGACAAGAGGCUAUGAUCCUGAAUGCAUCCUUGG
CAAAC-3′
4-Pyridyl Modified:
(SEQ ID NO: 315):
5′-AGGAGAAUCAUGAGUUAUCUGUGUAAGGAACCAAAGCCAUGCUUAU
CAAAC-3′

### Example 35: Reverse Chimera Linkers

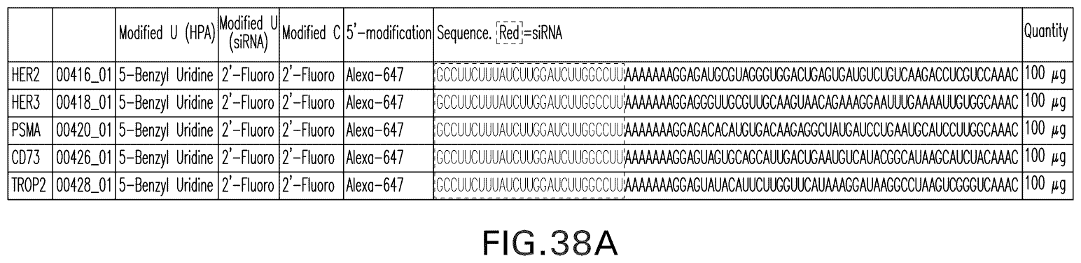

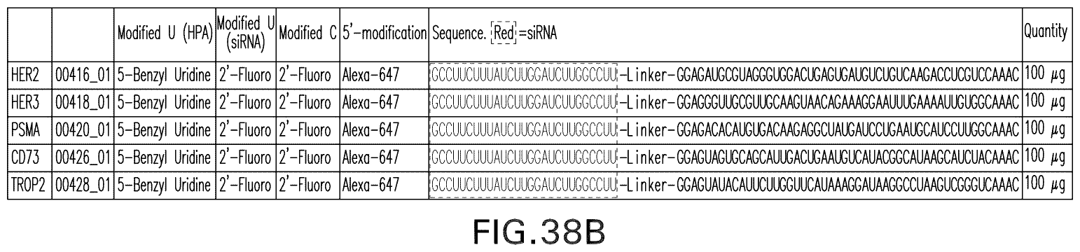

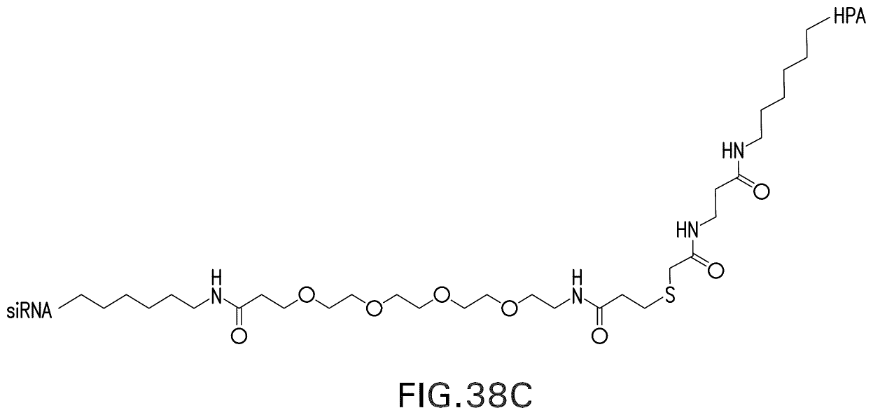

Standard linkage is 3′ end of an aptamer linked to 5′ of an siRNA. Here we provide an example of the 3′end of the siRNA linked to the 5′end of the aptamer. Linked via poly-adenosine linkage. siRNA is the guide strand (FIG. 38A). Provided is another example of a reverse chimera structure using an alternative linker shown in (FIG. 38C). However, alternative linkers as previously described can be used in place here.

### Example 36: NR4A1 is Linked to CD8⁺ T Cell Dysfunction

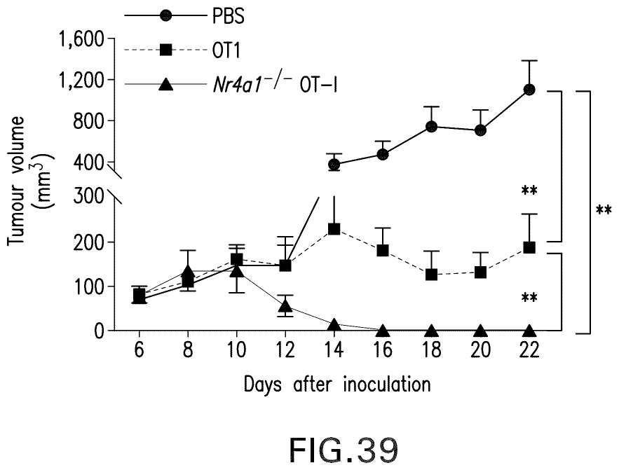

CD45.1⁺CD45.2⁺ (B6SJL×C57BL6) congenic mice were subcutaneously injected with OVA-expressing EL4 cells (E.G7 lymphoma) cells (5×10⁵ cells per mouse) in one flank. Six days later, PBS, wild-type or Nr4a1^{-/-} OT-I cells (3×10⁶ cells per mouse) were adoptively transferred into mice intravenously. Tumor sizes were monitored after adoptive transfer. To assess tumor-infiltrating donor T cells, mice were euthanized 6 days after T cell transfer. Donor-derived T cells were collected from tumor, draining lymph nodes and spleens, and subjected to flow cytometry analysis. Adoptive transfer of Nr4a1^{-/-} transgenic CD8⁺ T recognizing OVA₂₅₇₋₂₆₄ peptide (OT-I) cells into E.G7 tumor cell-bearing mice nearly eliminated tumors, in contrast with wild-type OT-I cells and a PBS control group. This data supports that NR4A1 is linked to CD8⁺ T cell dysfunction (Liu, X., et al. Nature 2019)(FIG. 39).

### Example 37: Exhausted CD8+ T Cells

Dysfunctional, or exhausted CD8⁺ T cells arise in the settings of chronic viral infection or cancer when persistent exposure to antigen leads to prolonged T cell receptor (TCR) signaling. In the exhausted state, T cell effector functions are impaired and manifest as decreased proliferative capacity, reduced cytolytic function and effector cytokine production, and altered in gene expression and metabolism. Notably, exhausted T cells upregulate multiple inhibitory receptors that include but are not limited to these immune checkpoint proteins: PD-1, CTLA-4, TIM-3, LAG-3, TIGIT, 2B4/CD244 and others. While activated effector T cells also transiently express immune checkpoint proteins, expression level increase and are sustained on exhausted T cell subsets. Transcription factors such as TOX and NR4A1 have been described as master regulators of exhaust (FIG. 40).

### Example 38: In Vivo Inhibition of UBB and UBC mRNA by the UBB-UBC Dual Targeting siRNA

Male NSG mice are injected subcutaneously (HCT116) or intrasplenically (mHCT116) with human HCT116 CRC tumor cells to disseminate LM, whereas experimental controls receive saline. Huot et al. demonstrated elevated ubiquitin expression in this model (Huot et al., Dis Models & Mech, 13:1754-8403 (2020)).

Mice will be treated with the dual UBB-UBC targeting siRNAs conjugated to EPCAM aptamer, EpCAM-scrambled siRNA, or vehicle by intraperitoneal injection of 0.1 ml of the indicated solution. Mice will be treated with a dose of dual targeting siRNA sufficient to inhibit expression of UBB and UBC by at least 10%, at least 15%, at least 20%, at least 25%, at least 30%, at least 40%, at least 50%, at least 60%, at least 70%, at least 80% or at least 90% or more, for at least 5, more preferably 7, 10, 14, or 18 days. Alternatively, mice will be dosed multiple times in order to inhibit expression of UBB and UBC by at least 10%, at least 15%, at least 20%, at least 25%, at least 30%, at least 40%, at least 50%, at least 60%, at least 70%, at least 80% or at least 90% or more, for at least 5, more preferably 7, 10, 14, or 18 days. All the mice are sacrificed on day 18, and tumors are collected for quantitation.

### Example 39: In Vivo Impact of UBB and UBC mRNA Inhibition on Tumor Size

To assess the impact of a compound comprising dual targeting siRNA conjugated to EPCAM aptamer on tumor growth in vivo, subcutaneous HCT-116 xenografts will be established in athymic nu/nu male mice. The compound will be injected intraperitoneally to tumor-bearing mice every other day for 1 week and every day for the following two weeks. Control mice will be injected intraperitoneally with equivalent volume of PBS or EpCAM-scrambled siRNA. All the mice are sacrificed on day 21, and tumors are collected for quantitation.

In certain embodiments, the invention provides pharmaceutical compositions containing a dual targeting siRNA agent, as described herein, and a pharmaceutically acceptable carrier.

The pharmaceutical compositions featured herein are administered in dosages sufficient to inhibit expression of the target genes. In general, a suitable dose of siRNA will be in the range of 0.01 to 200.0 milligrams per kilogram body weight of the recipient per day, generally in the range of 1 to 50 mg per kilogram body weight per day.

The pharmaceutical composition may be administered once daily, or the siRNA may be administered as two, three, or more sub-doses at appropriate intervals throughout the day or even using continuous infusion or delivery through a controlled release formulation. In that case, the siRNA contained in each sub-dose must be correspondingly smaller in order to achieve the total daily dosage. The dosage unit can also be compounded for delivery over several days, e.g., using a conventional sustained release formulation which provides sustained release of the siRNA over a several day period. Sustained release formulations are well known in the art and are particularly useful for delivery of agents at a particular site, such as could be used with the agents of the present invention. In this embodiment, the dosage unit contains a corresponding multiple of the daily dose.

Many modifications and variations of this invention can be made without departing from its spirit and scope, as will be apparent to those skilled in the art. The invention is defined by the terms of the appended claims, along with the full scope of equivalents to which such claims are entitled. The specific embodiments described herein, including the following examples, are offered by way of example only, and do not by their details limit the scope of the invention.

All references cited herein are incorporated by reference to the same extent as if each individual publication, database entry (e.g., Genbank sequences or GeneID entries), patent application, or patent, was specifically and individually indicated to be incorporated by reference. This statement of incorporation by reference is intended by Applicants, pursuant to 37 C.F.R. § 1.57(b)(1), to relate to each and every individual publication, database entry (e.g., Genbank sequences or GeneID entries), patent application, or patent, each of which is clearly identified in compliance with 37 C.F.R. § 1.57(b)(2), even if such citation is not immediately adjacent to a dedicated statement of incorporation by reference. The inclusion of dedicated statements of incorporation by reference, if any, within the specification does not in any way weaken this general statement of incorporation by reference. Citation of the references herein is not intended as an admission that the reference is pertinent prior art, nor does it constitute any admission as to the contents or date of these publications or documents.

The present invention is not to be limited in scope by the specific embodiments described herein. Indeed, various modifications of the invention in addition to those described herein will become apparent to those skilled in the art from the foregoing description and the accompanying figures. Such modifications are intended to fall within the scope of the appended claims.

The foregoing written specification is considered to be sufficient to enable one skilled in the art to practice the invention. Various modifications of the invention in addition to those shown and described herein will become apparent to those skilled in the art from the foregoing description and fall within the scope of the appended claims.

1. A siRNA construct that is processed by cellular RNAi machinery to produce one or more siRNA molecules wherein each molecule specifically inhibits expression of two or more different genes, and wherein the siRNA construct further comprises siRNA sequence (SEQ ID NO 97).

2. Construct according to claim 1 wherein the siRNA molecules bind more than one site in each different gene.

3. (canceled)

4. An aptamer-siRNA chimera comprising an aptamer that specifically binds at least one target protein and an siRNA construct that is processed by cellular RNAi machinery to produce one or more siRNAs wherein at least one siRNA specifically inhibits expression of two or more different genes.

5. A chimera according to claim 4 wherein the different genes comprise (a) BCL2 and STAT3; (b) BCL2 and MYC; (c) BCL2 and SYK; (d) BCL2 and CCNE2; (e) CCND1 and CCNE2; (f) CCND2 and BIRC5; (g) CCND1 and EGFR; (h) UBB and UBC: (i) NR4A1 and NR4A2; (i) NR4A1 and NR4A3; (k) NR4A2 and NR4A3; (l) ADORA2a and ADORA2b; (m) ADORA2a and ADORA1; (n) MAP2K1 and MAP2K2; (o) MAPK3 (ERK1) and MAPK1 (ERK2); (p) MAPK11 and MAPK14; (g) HIF1 and HIF2; (r) PFKFB3 and PFKFB4; (s) PFKFB3 and PFKFB2: (t) PLK1 and PLK4; (u) CDK11A and CDK11B; (v) CDK4 and CDK6; (w) PARP1 and PARP2; (x) MDM2 and MDM4; or (y) ATK1 and BATF.

6-31. (canceled)

32. An aptamer-siRNA chimera comprising:
a. first and second ends comprising an aptamer that specifically binds at least one target protein;
b. an siRNA construct between the first and second ends, wherein
the siRNA construct is processed by cellular RNAi machinery to produce one or more siRNAs wherein at least one siRNA specifically inhibits expression of two or more different genes.

33. The chimera according to claim 4 wherein the siRNA construct is processed by cellular RNAi machinery to produce two siRNAs that specifically inhibit three or more different genes.

34. An aptamer-siRNA chimera comprising:
a. first and second ends, wherein the first and second ends comprise an aptamer that specifically binds at least one target protein;
b. an siRNA construct between the first and second ends, wherein
the siRNA construct is processed by cellular RNAi machinery to produce one or more siRNAs wherein at least one siRNA specifically inhibits expression of two or more different genes.

35. The chimera according to claim 34 wherein said genes comprise UBB and UBC.

36. The chimera according to claim 4 further comprising unpaired linkers comprising two to four adenines between each aptamer and siRNA and between each siRNA.

37. The chimera according to claim 4, wherein the target protein comprises ERBB2, ERBB3, FOLH1, CD44, EPCAM, FOLH1, PSCA, PDCD1, TACSTD2, NT5E, PDCD1, CTLA4, LAG3 or HAVCR2.

38. The chimera according to claim 4, wherein the construct comprises the nucleic acid sequence (SEQ ID NO:6) or (SEQ ID NO:22) or (SEQ ID NO:21).

39. An aptamer-siRNA chimera comprising:
a. first and second ends, wherein the first and second ends comprise an aptamer that specifically bind EPCAM;
b. an siRNA construct between the first and second ends, wherein
the siRNA construct is processed by cellular RNAi machinery to produce a first siRNA that inhibits expression of two genes, and a second siRNA that inhibits expression of two different genes; and
unpaired linkers comprising two to four adenines between each aptamer and siRNA and between each siRNA.

40. The chimera of claim 39, wherein the first siRNA inhibits expression of both UBB and UBC.

41. The chimera of claim 40, wherein the second siRNA inhibits expression of two different oncogenes genes selected from the group comprising BCL2, STAT3, MYC, SYK, CCNE2, CCND1, CCND2, BIRC5, EGFR, UBB, UBC, NR4A1, NR4A2, NR4A1, NR4A3, ADORA2a, ADORA2b, ADORA1, MAP2K1, MAP2K2, MAPK3 (ERK1), MAPK1 (ERK2), HIF1, HIF2, PFKFB3, PFKFB4, PLK1, PLK4, CDK11A, CDK11B, CDK4, CDK6, PARP1, or PARP2.

42. The chimera according to claim 39, wherein the genes further comprise one or more genes selected from the group comprising BCL2, STAT3, MYC, SYK, CCNE2, CCND1, CCND2, BIRC5, EGFR, UBB, UBC, NR4A1, NR4A2, NR4A1, NR4A3, ADORA2a, ADORA2b, ADORA1, MAP2K1, MAP2K2, MAPK3 (ERK1), MAPK1 (ERK2), HIF1, HIF2, PFKFB3, PFKFB4, PLK1, PLK4, CDK11A, CDK11B, CDK4, CDK6, PARP1, or PARP2.

43. A pharmaceutical composition comprising an effective amount of a chimera according to claim 4 plus a pharmaceutically acceptable carrier.

44. A method for reducing tumor burden in a subject in need thereof comprising, administering to the subject an effective amount of the pharmaceutical composition according to claim 43.

45. A method for treating cancer and reducing tumor burden in a subject in need thereof comprising, administering to the subject a dual UBB/UBC inhibitor.

46. The method of claim 45, wherein the dual UBB/UBC inhibitor is selected from group comprising siRNA, mRNA, small molecule, antibody, aptamer, and antisense oligonucleotide.

47. The method of claim 46 wherein the dual UBB/UBC inhibitor is siRNA.

48. The method of claim 47 further comprising a ligand conjugated to the dual UBB/UBC inhibitor that specifically binds a cell surface protein expressed by the tumor.

49. The method of claim 48 wherein the ligand is an aptamer.

50. The method of claim 49 wherein the cell surface protein is EPCAM.

51. The method of claim 44, wherein the target protein of the pharmaceutical composition comprises ERBB2, ERBB3, FOLH1, CD44, EPCAM, FOLH1, PSCA, PDCD1, TACSTD2, NT5E, PDCD1, CTLA4, LAG3 or HAVCR2.

52-55. (canceled)


SEQUENCE LISTING (422 sequences)

SEQ ID NO: 1 - RNA, 19 nt, Artificial Sequence
  Description of Artificial Sequence Synthetic oligonucleotide
caccauugaugucuacaug

SEQ ID NO: 2 - RNA, 19 nt, Artificial Sequence
  Description of Artificial Sequence Synthetic oligonucleotide
cagcaacuucuucaugaac

SEQ ID NO: 3 - RNA, 19 nt, Artificial Sequence
  Description of Artificial Sequence Synthetic oligonucleotide
caucuucaaggagaacaaa

SEQ ID NO: 4 - RNA, 19 nt, Artificial Sequence
  Description of Artificial Sequence Synthetic oligonucleotide
ugccaggaggcggcagcuc

SEQ ID NO: 5 - RNA, 19 nt, Artificial Sequence
  Description of Artificial Sequence Synthetic oligonucleotide
ugcaguaucuggccccaga

SEQ ID NO: 6 - RNA, 19 nt, Artificial Sequence
  Description of Artificial Sequence Synthetic oligonucleotide
cuacauagggaucugaaac

SEQ ID NO: 7 - RNA, 19 nt, Artificial Sequence
  Description of Artificial Sequence Synthetic oligonucleotide
uuugccaguggcaguccau

SEQ ID NO: 8 - RNA, 19 nt, Artificial Sequence
  Description of Artificial Sequence Synthetic oligonucleotide
gaauguaagaaagcucagc

SEQ ID NO: 9 - RNA, 19 nt, Artificial Sequence
  Description of Artificial Sequence Synthetic oligonucleotide
gucuacuucaacuucuuug

SEQ ID NO: 10 - RNA, 19 nt, Artificial Sequence
  Description of Artificial Sequence Synthetic oligonucleotide
ccucacgcagagcuccauc

SEQ ID NO: 11 - RNA, 19 nt, Artificial Sequence
  Description of Artificial Sequence Synthetic oligonucleotide
ccucacccagagcuccauc

SEQ ID NO: 12 - RNA, 19 nt, Artificial Sequence
  Description of Artificial Sequence Synthetic oligonucleotide
caugguguacuucaacuuc

SEQ ID NO: 13 - RNA, 19 nt, Artificial Sequence
  Description of Artificial Sequence Synthetic oligonucleotide
cauggucuacuucaacuuc

SEQ ID NO: 14 - RNA, 19 nt, Artificial Sequence
  Description of Artificial Sequence Synthetic oligonucleotide
guguacuucaacuucuuug

SEQ ID NO: 15 - RNA, 19 nt, Artificial Sequence
  Description of Artificial Sequence Synthetic oligonucleotide
gucuacuucaacuucuuug

SEQ ID NO: 16 - RNA, 19 nt, Artificial Sequence
  Description of Artificial Sequence Synthetic oligonucleotide
aauccggaaccagaucaua

SEQ ID NO: 17 - RNA, 19 nt, Artificial Sequence
  Description of Artificial Sequence Synthetic oligonucleotide
cauccggaaccagaucauc

SEQ ID NO: 18 - RNA, 19 nt, Artificial Sequence
  Description of Artificial Sequence Synthetic oligonucleotide
guacaucgugggcuucuau

SEQ ID NO: 19 - RNA, 19 nt, Artificial Sequence
  Description of Artificial Sequence Synthetic oligonucleotide
guacaucgugggcuucuac

SEQ ID NO: 20 - RNA, 19 nt, Artificial Sequence
  Description of Artificial Sequence Synthetic oligonucleotide
caagcccuccaacauccua

SEQ ID NO: 21 - RNA, 19 nt, Artificial Sequence
  Description of Artificial Sequence Synthetic oligonucleotide
gaagcccuccaacauccuc

SEQ ID NO: 22 - RNA, 19 nt, Artificial Sequence
  Description of Artificial Sequence Synthetic oligonucleotide
gaucaagcucugugacuuu

SEQ ID NO: 23 - RNA, 19 nt, Artificial Sequence
  Description of Artificial Sequence Synthetic oligonucleotide
gaucaagcugugugacuuc

SEQ ID NO: 24 - RNA, 19 nt, Artificial Sequence
  Description of Artificial Sequence Synthetic oligonucleotide
ucgacuccauggccaacuc

SEQ ID NO: 25 - RNA, 19 nt, Artificial Sequence
  Description of Artificial Sequence Synthetic oligonucleotide
cauggccaacuccuucgug

SEQ ID NO: 26 - RNA, 19 nt, Artificial Sequence
  Description of Artificial Sequence Synthetic oligonucleotide
ugagcaaugaccauaucug

SEQ ID NO: 27 - RNA, 19 nt, Artificial Sequence
  Description of Artificial Sequence Synthetic oligonucleotide
ucagcaaugaccauaucug

SEQ ID NO: 28 - RNA, 19 nt, Artificial Sequence
  Description of Artificial Sequence Synthetic oligonucleotide
cuacuuccucuaccagauc

SEQ ID NO: 29 - RNA, 19 nt, Artificial Sequence
  Description of Artificial Sequence Synthetic oligonucleotide
cuauuuucucuaccagauc

SEQ ID NO: 30 - RNA, 19 nt, Artificial Sequence
  Description of Artificial Sequence Synthetic oligonucleotide
ccaagggcuauaccaaguc

SEQ ID NO: 31 - RNA, 19 nt, Artificial Sequence
  Description of Artificial Sequence Synthetic oligonucleotide
ccaagggcuacaccaaguc

SEQ ID NO: 32 - RNA, 19 nt, Artificial Sequence
  Description of Artificial Sequence Synthetic oligonucleotide
gucugugggcugcauucug

SEQ ID NO: 33 - RNA, 19 nt, Artificial Sequence
  Description of Artificial Sequence Synthetic oligonucleotide
gucuguaggcugcauucug

SEQ ID NO: 34 - RNA, 19 nt, Artificial Sequence
  Description of Artificial Sequence Synthetic oligonucleotide
cucuaaccggcccaucuuc

SEQ ID NO: 35 - RNA, 19 nt, Artificial Sequence
  Description of Artificial Sequence Synthetic oligonucleotide
cucuaacaggcccaucuuc

SEQ ID NO: 36 - RNA, 19 nt, Artificial Sequence
  Description of Artificial Sequence Synthetic oligonucleotide
ggaggaccugaauuguauc

SEQ ID NO: 37 - RNA, 19 nt, Artificial Sequence
  Description of Artificial Sequence Synthetic oligonucleotide
agaagaccugaauuguaua

SEQ ID NO: 38 - RNA, 19 nt, Artificial Sequence
  Description of Artificial Sequence Synthetic oligonucleotide
gaaagccuuggaugguuuu

SEQ ID NO: 39 - RNA, 19 nt, Artificial Sequence
  Description of Artificial Sequence Synthetic oligonucleotide
uacucaggccgccaucaag

SEQ ID NO: 40 - RNA, 19 nt, Artificial Sequence
  Description of Artificial Sequence Synthetic oligonucleotide
agaccuacaucuccaagaa

SEQ ID NO: 41 - RNA, 19 nt, Artificial Sequence
  Description of Artificial Sequence Synthetic oligonucleotide
gugggacuccuaauuacau

SEQ ID NO: 42 - RNA, 19 nt, Artificial Sequence
  Description of Artificial Sequence Synthetic oligonucleotide
caagaaaacagaugaaauu

SEQ ID NO: 43 - RNA, 19 nt, Artificial Sequence
  Description of Artificial Sequence Synthetic oligonucleotide
guuucgucgaaagccucuc

SEQ ID NO: 44 - RNA, 19 nt, Artificial Sequence
  Description of Artificial Sequence Synthetic oligonucleotide
ccaagagugccaacuacug

SEQ ID NO: 45 - RNA, 19 nt, Artificial Sequence
  Description of Artificial Sequence Synthetic oligonucleotide
agcgcacagugcagaaaaa

SEQ ID NO: 46 - RNA, 19 nt, Artificial Sequence
  Description of Artificial Sequence Synthetic oligonucleotide
ugcuguguguggggacaac

SEQ ID NO: 47 - RNA, 19 nt, Artificial Sequence
  Description of Artificial Sequence Synthetic oligonucleotide
cgcuguguguggggacaac

SEQ ID NO: 48 - RNA, 19 nt, Artificial Sequence
  Description of Artificial Sequence Synthetic oligonucleotide
augugagggcugcugcaag

SEQ ID NO: 49 - RNA, 19 nt, Artificial Sequence
  Description of Artificial Sequence Synthetic oligonucleotide
cugugagggcugcugcaaa

SEQ ID NO: 50 - RNA, 19 nt, Artificial Sequence
  Description of Artificial Sequence Synthetic oligonucleotide
cugcaagggcuucuucaag

SEQ ID NO: 51 - RNA, 19 nt, Artificial Sequence
  Description of Artificial Sequence Synthetic oligonucleotide
cugcaaaggcuucuuuaag

SEQ ID NO: 52 - RNA, 19 nt, Artificial Sequence
  Description of Artificial Sequence Synthetic oligonucleotide
gggcugcaagggcuucuuc

SEQ ID NO: 53 - RNA, 19 nt, Artificial Sequence
  Description of Artificial Sequence Synthetic oligonucleotide
gggcugcaaaggcuucuuu

SEQ ID NO: 54 - RNA, 19 nt, Artificial Sequence
  Description of Artificial Sequence Synthetic oligonucleotide
gcgcacagugcagaaaaac

SEQ ID NO: 55 - RNA, 19 nt, Artificial Sequence
  Description of Artificial Sequence Synthetic oligonucleotide
gcgcacagugcaaaaaaac

SEQ ID NO: 56 - RNA, 19 nt, Artificial Sequence
  Description of Artificial Sequence Synthetic oligonucleotide
ugcuguguguggggacaac

SEQ ID NO: 57 - RNA, 19 nt, Artificial Sequence
  Description of Artificial Sequence Synthetic oligonucleotide
cgcuguguguggggacaac

SEQ ID NO: 58 - RNA, 19 nt, Artificial Sequence
  Description of Artificial Sequence Synthetic oligonucleotide
ugccgugugcggggacaac

SEQ ID NO: 59 - RNA, 19 nt, Artificial Sequence
  Description of Artificial Sequence Synthetic oligonucleotide
cugcaagggcuucuucaag

SEQ ID NO: 60 - RNA, 19 nt, Artificial Sequence
  Description of Artificial Sequence Synthetic oligonucleotide
cugcaaaggcuucuuuaag

SEQ ID NO: 61 - RNA, 19 nt, Artificial Sequence
  Description of Artificial Sequence Synthetic oligonucleotide
cugcaagggcuuuuucaag

SEQ ID NO: 62 - RNA, 19 nt, Artificial Sequence
  Description of Artificial Sequence Synthetic oligonucleotide
gggcugcaagggcuucuuc

SEQ ID NO: 63 - RNA, 19 nt, Artificial Sequence
  Description of Artificial Sequence Synthetic oligonucleotide
gggcugcaaaggcuucuuu

SEQ ID NO: 64 - RNA, 19 nt, Artificial Sequence
  Description of Artificial Sequence Synthetic oligonucleotide
gggcugcaagggcuuuuuc

SEQ ID NO: 65 - RNA, 19 nt, Artificial Sequence
  Description of Artificial Sequence Synthetic oligonucleotide
gcgcacagugcagaaaaac

SEQ ID NO: 66 - RNA, 19 nt, Artificial Sequence
  Description of Artificial Sequence Synthetic oligonucleotide
gcgcacagugcaaaaaaac

SEQ ID NO: 67 - RNA, 19 nt, Artificial Sequence
  Description of Artificial Sequence Synthetic oligonucleotide
gagaacagugcagaaaaau

SEQ ID NO: 68 - RNA, 19 nt, Artificial Sequence
  Description of Artificial Sequence Synthetic oligonucleotide
acugcccuguggacaagag

SEQ ID NO: 69 - RNA, 19 nt, Artificial Sequence
  Description of Artificial Sequence Synthetic oligonucleotide
acugcccaguggacaagcg

SEQ ID NO: 70 - RNA, 19 nt, Artificial Sequence
  Description of Artificial Sequence Synthetic oligonucleotide
acugcccaguagacaagag

SEQ ID NO: 71 - RNA, 19 nt, Artificial Sequence
  Description of Artificial Sequence Synthetic oligonucleotide
uucaaugaugauauucaag

SEQ ID NO: 72 - RNA, 19 nt, Artificial Sequence
  Description of Artificial Sequence Synthetic oligonucleotide
auccaacaauagaggauuc

SEQ ID NO: 73 - RNA, 19 nt, Artificial Sequence
  Description of Artificial Sequence Synthetic oligonucleotide
ucauuuuguggacgaauau

SEQ ID NO: 74 - RNA, 19 nt, Artificial Sequence
  Description of Artificial Sequence Synthetic oligonucleotide
guggugugagggcuccagc

SEQ ID NO: 75 - RNA, 19 nt, Artificial Sequence
  Description of Artificial Sequence Synthetic oligonucleotide
cuuacaacacacaacuuug

SEQ ID NO: 76 - DNA, 25 bp, Artificial Sequence
  Description of Artificial Sequence Synthetic oligonucleotide
  Description of Combined DNA/RNA Molecule Synthetic oligonucleotide
aauugaggugcuuccuuaauuguga

SEQ ID NO: 77 - RNA, 27 nt, Artificial Sequence
  Description of Artificial Sequence Synthetic oligonucleotide
ucacaaucagggaagcaucacaauugg

SEQ ID NO: 78 - RNA, 21 nt, Artificial Sequence
  Description of Artificial Sequence Synthetic oligonucleotide
aauugaggugcuuccuuaauu

SEQ ID NO: 79 - RNA, 21 nt, Artificial Sequence
  Description of Artificial Sequence Synthetic oligonucleotide
ucagggaagcaucacaauugg

SEQ ID NO: 80 - DNA, 25 bp, Artificial Sequence
  Description of Artificial Sequence Synthetic oligonucleotide
  Description of Combined DNA/RNA Molecule Synthetic oligonucleotide
auuugaggcaguuuacauuucggtg

SEQ ID NO: 81 - RNA, 27 nt, Artificial Sequence
  Description of Artificial Sequence Synthetic oligonucleotide
caccgaaauguucacuuccucaaguuc

SEQ ID NO: 82 - RNA, 21 nt, Artificial Sequence
  Description of Artificial Sequence Synthetic oligonucleotide
auuugaggcaguuuacauuuc

SEQ ID NO: 83 - RNA, 21 nt, Artificial Sequence
  Description of Artificial Sequence Synthetic oligonucleotide
aauguucacuuccucaaguuc

SEQ ID NO: 84 - DNA, 25 bp, Artificial Sequence
  Description of Artificial Sequence Synthetic oligonucleotide
  Description of Combined DNA/RNA Molecule Synthetic oligonucleotide
uuuccauuugucuuugaucuuugtt

SEQ ID NO: 85 - RNA, 27 nt, Artificial Sequence
  Description of Artificial Sequence Synthetic oligonucleotide
aacaaagaucacauauaaauggaaggc

SEQ ID NO: 86 - RNA, 21 nt, Artificial Sequence
  Description of Artificial Sequence Synthetic oligonucleotide
uuuccauuugucuuugaucuu

SEQ ID NO: 87 - RNA, 21 nt, Artificial Sequence
  Description of Artificial Sequence Synthetic oligonucleotide
gaucacauauaaauggaaggc

SEQ ID NO: 88 - DNA, 25 bp, Artificial Sequence
  Description of Artificial Sequence Synthetic oligonucleotide
  Description of Combined DNA/RNA Molecule Synthetic oligonucleotide
uuccucaaguuuggaagcagccutg

SEQ ID NO: 89 - RNA, 27 nt, Artificial Sequence
  Description of Artificial Sequence Synthetic oligonucleotide
caaggcugauucuaaacuggaagaaaa

SEQ ID NO: 90 - DNA, 25 bp, Artificial Sequence
  Description of Artificial Sequence Synthetic oligonucleotide
  Description of Combined DNA/RNA Molecule Synthetic oligonucleotide
accaucagugacguaagcaaaagaa

SEQ ID NO: 91 - RNA, 27 nt, Artificial Sequence
  Description of Artificial Sequence Synthetic oligonucleotide
uucuuuugcuuaagucagagauggaag

SEQ ID NO: 92 - DNA, 25 bp, Artificial Sequence
  Description of Artificial Sequence Synthetic oligonucleotide
  Description of Combined DNA/RNA Molecule Synthetic oligonucleotide
auuccaaugccauccacuuucagtc

SEQ ID NO: 93 - RNA, 27 nt, Artificial Sequence
  Description of Artificial Sequence Synthetic oligonucleotide
gacugaaagugcuuggaaauggaaugg

SEQ ID NO: 94 - DNA, 25 bp, Artificial Sequence
  Description of Artificial Sequence Synthetic oligonucleotide
  Description of Combined DNA/RNA Molecule Synthetic oligonucleotide
uccauuuacagacugacacuuucca

SEQ ID NO: 95 - RNA, 27 nt, Artificial Sequence
  Description of Artificial Sequence Synthetic oligonucleotide
uggaaaguuacagacuguaaauagagu

SEQ ID NO: 96 - DNA, 21 bp, Artificial Sequence
  Description of Artificial Sequence Synthetic oligonucleotide
aaggccaagatccaagataaa

SEQ ID NO: 97 - DNA, 21 bp, Artificial Sequence
  Description of Artificial Sequence Synthetic oligonucleotide
aagaggtggtatgcagatctt

SEQ ID NO: 98 - DNA, 19 bp, Artificial Sequence
  Description of Artificial Sequence Synthetic oligonucleotide
aaatgtgaaggccaagatc

SEQ ID NO: 99 - DNA, 19 bp, Artificial Sequence
  Description of Artificial Sequence Synthetic oligonucleotide
aatgtgaaggccaagatcc

SEQ ID NO: 100 - DNA, 19 bp, Artificial Sequence
  Description of Artificial Sequence Synthetic oligonucleotide
atgtgaaggccaagatcca

SEQ ID NO: 101 - DNA, 19 bp, Artificial Sequence
  Description of Artificial Sequence Synthetic oligonucleotide
tgtgaaggccaagatccaa

SEQ ID NO: 102 - DNA, 19 bp, Artificial Sequence
  Description of Artificial Sequence Synthetic oligonucleotide
gtgaaggccaagatccaag

SEQ ID NO: 103 - DNA, 19 bp, Artificial Sequence
  Description of Artificial Sequence Synthetic oligonucleotide
tgaaggccaagatccaaga

SEQ ID NO: 104 - DNA, 19 bp, Artificial Sequence
  Description of Artificial Sequence Synthetic oligonucleotide
gaaggccaagatccaagat

SEQ ID NO: 105 - DNA, 19 bp, Artificial Sequence
  Description of Artificial Sequence Synthetic oligonucleotide
aaggccaagatccaagata

SEQ ID NO: 106 - DNA, 19 bp, Artificial Sequence
  Description of Artificial Sequence Synthetic oligonucleotide
aggccaagatccaagataa

SEQ ID NO: 107 - DNA, 19 bp, Artificial Sequence
  Description of Artificial Sequence Synthetic oligonucleotide
ggccaagatccaagataaa

SEQ ID NO: 108 - DNA, 19 bp, Artificial Sequence
  Description of Artificial Sequence Synthetic oligonucleotide
gccaagatccaagataaag

SEQ ID NO: 109 - DNA, 19 bp, Artificial Sequence
  Description of Artificial Sequence Synthetic oligonucleotide
ccaagatccaagataaaga

SEQ ID NO: 110 - DNA, 19 bp, Artificial Sequence
  Description of Artificial Sequence Synthetic oligonucleotide
caagatccaagataaagaa

SEQ ID NO: 111 - DNA, 19 bp, Artificial Sequence
  Description of Artificial Sequence Synthetic oligonucleotide
aagatccaagataaagaag

SEQ ID NO: 112 - DNA, 19 bp, Artificial Sequence
  Description of Artificial Sequence Synthetic oligonucleotide
agatccaagataaagaagg

SEQ ID NO: 113 - DNA, 19 bp, Artificial Sequence
  Description of Artificial Sequence Synthetic oligonucleotide
gatccaagataaagaaggc

SEQ ID NO: 114 - DNA, 19 bp, Artificial Sequence
  Description of Artificial Sequence Synthetic oligonucleotide
atccaagataaagaaggca

SEQ ID NO: 115 - DNA, 19 bp, Artificial Sequence
  Description of Artificial Sequence Synthetic oligonucleotide
tccaagataaagaaggcat

SEQ ID NO: 116 - DNA, 19 bp, Artificial Sequence
  Description of Artificial Sequence Synthetic oligonucleotide
ccaagataaagaaggcatc

SEQ ID NO: 117 - DNA, 21 bp, Unknown
  Description of Unknown UBB_1 sequence
caggatcctggtatccgctaa

SEQ ID NO: 118 - DNA, 21 bp, Unknown
  Description of Unknown UBB_2 sequence
atggcattactctgcactata

SEQ ID NO: 119 - DNA, 21 bp, Unknown
  Description of Unknown UBB_3 sequence
ccaacttaagtttagaaatta

SEQ ID NO: 120 - DNA, 21 bp, Unknown
  Description of Unknown UBB_4 sequence
gaggctcatctttgcaggcaa

SEQ ID NO: 121 - DNA, 19 bp, Artificial Sequence
  Description of Artificial Sequence Synthetic oligonucleotide
gaacaaccggcaaatagat

SEQ ID NO: 122 - DNA, 19 bp, Artificial Sequence
  Description of Artificial Sequence Synthetic oligonucleotide
gcaatacgcgaagacataa

SEQ ID NO: 123 - DNA, 19 bp, Artificial Sequence
  Description of Artificial Sequence Synthetic oligonucleotide
gaaagacggaccataacat

SEQ ID NO: 124 - DNA, 19 bp, Artificial Sequence
  Description of Artificial Sequence Synthetic oligonucleotide
gaagaaccacgaagactta

SEQ ID NO: 125 - DNA, 19 bp, Artificial Sequence
  Description of Artificial Sequence Synthetic oligonucleotide
gtaggacgcacaaactaaa

SEQ ID NO: 126 - DNA, 19 bp, Artificial Sequence
  Description of Artificial Sequence Synthetic oligonucleotide
ggacagatcgctaaacaaa

SEQ ID NO: 127 - DNA, 19 bp, Artificial Sequence
  Description of Artificial Sequence Synthetic oligonucleotide
ggccaagatccaggataaa

SEQ ID NO: 128 - DNA, 19 bp, Artificial Sequence
  Description of Artificial Sequence Synthetic oligonucleotide
ggccaagatccaggataag

SEQ ID NO: 129 - DNA, 19 bp, Artificial Sequence
  Description of Artificial Sequence Synthetic oligonucleotide
ggcaaagatccaagataag

SEQ ID NO: 130 - RNA, 19 nt, Artificial Sequence
  Description of Artificial Sequence Synthetic oligonucleotide
gccguacucuuucugacua

SEQ ID NO: 131 - RNA, 19 nt, Artificial Sequence
  Description of Artificial Sequence Synthetic oligonucleotide
guaugcagaucuucgugaa

SEQ ID NO: 132 - RNA, 19 nt, Artificial Sequence
  Description of Artificial Sequence Synthetic oligonucleotide
gaccaucacucuggaggug

SEQ ID NO: 133 - RNA, 19 nt, Artificial Sequence
  Description of Artificial Sequence Synthetic oligonucleotide
cccagugacaccaucgaaa

SEQ ID NO: 134 - RNA, 19 nt, Artificial Sequence
  Description of Artificial Sequence Synthetic oligonucleotide
gugaagacccugacuggua

SEQ ID NO: 135 - RNA, 19 nt, Artificial Sequence
  Description of Artificial Sequence Synthetic oligonucleotide
aagcaaagauccaggacaa

SEQ ID NO: 136 - RNA, 19 nt, Artificial Sequence
  Description of Artificial Sequence Synthetic oligonucleotide
gaagauggacgcacccugu

SEQ ID NO: 137 - RNA, 19 nt, Artificial Sequence
  Description of Artificial Sequence Synthetic oligonucleotide
guaagaccaucacucucga

SEQ ID NO: 138 - DNA, 19 bp, Artificial Sequence
  Description of Artificial Sequence Synthetic oligonucleotide
caagaccatcacccttgag

SEQ ID NO: 139 - DNA, 19 bp, Artificial Sequence
  Description of Artificial Sequence Synthetic oligonucleotide
tgcagatcttcgtgaagac

SEQ ID NO: 140 - DNA, 19 bp, Artificial Sequence
  Description of Artificial Sequence Synthetic oligonucleotide
agcccagtgacaccatcga

SEQ ID NO: 141 - DNA, 19 bp, Artificial Sequence
  Description of Artificial Sequence Synthetic oligonucleotide
gactacaacatccagaaag

SEQ ID NO: 142 - DNA, 19 bp, Artificial Sequence
  Description of Artificial Sequence Synthetic oligonucleotide
ctacaacatccagaaagag

SEQ ID NO: 143 - DNA, 19 bp, Artificial Sequence
  Description of Artificial Sequence Synthetic oligonucleotide
tgactacaacatccagaaa

SEQ ID NO: 144 - DNA, 19 bp, Artificial Sequence
  Description of Artificial Sequence Synthetic oligonucleotide
agtgacaccatcgaaaatg

SEQ ID NO: 145 - DNA, 19 bp, Artificial Sequence
  Description of Artificial Sequence Synthetic oligonucleotide
aggcaaagatccaagataa

SEQ ID NO: 146 - DNA, 19 bp, Artificial Sequence
  Description of Artificial Sequence Synthetic oligonucleotide
ggcaaagatccaagacaag

SEQ ID NO: 147 - DNA, 19 bp, Artificial Sequence
  Description of Artificial Sequence Synthetic oligonucleotide
caaggcaaagatccaagac

SEQ ID NO: 148 - DNA, 19 bp, Artificial Sequence
  Description of Artificial Sequence Synthetic oligonucleotide
aggcaaagatccaagacaa

SEQ ID NO: 149 - DNA, 19 bp, Artificial Sequence
  Description of Artificial Sequence Synthetic oligonucleotide
caggataaggaaggcattc

SEQ ID NO: 150 - DNA, 19 bp, Artificial Sequence
  Description of Artificial Sequence Synthetic oligonucleotide
caggacaaggaaggcattc

SEQ ID NO: 151 - DNA, 19 bp, Artificial Sequence
  Description of Artificial Sequence Synthetic oligonucleotide
ggcaagcagctggaagatg

SEQ ID NO: 152 - DNA, 19 bp, Artificial Sequence
  Description of Artificial Sequence Synthetic oligonucleotide
ggaaagcagctggaagatg

SEQ ID NO: 153 - DNA, 19 bp, Artificial Sequence
  Description of Artificial Sequence Synthetic oligonucleotide
gactacaacatccagaagg

SEQ ID NO: 154 - DNA, 19 bp, Artificial Sequence
  Description of Artificial Sequence Synthetic oligonucleotide
tgactacaacatccagaag

SEQ ID NO: 155 - RNA, 19 nt, Unknown
  Description of Unknown U52 sequence
ggcaaagauccaggacaag

SEQ ID NO: 156 - RNA, 25 nt, Artificial Sequence
  Description of Artificial Sequence Synthetic oligonucleotide
ggccaagauccaagauaaagaaggc

SEQ ID NO: 157 - DNA, 19 bp, Artificial Sequence
  Description of Artificial Sequence Synthetic oligonucleotide
tgctgtgtgtggggacaac

SEQ ID NO: 158 - DNA, 19 bp, Artificial Sequence
  Description of Artificial Sequence Synthetic oligonucleotide
gggctgcaagggcttcttc

SEQ ID NO: 159 - DNA, 19 bp, Artificial Sequence
  Description of Artificial Sequence Synthetic oligonucleotide
gcgcacagtgcagaaaaac

SEQ ID NO: 160 - DNA, 19 bp, Artificial Sequence
  Description of Artificial Sequence Synthetic oligonucleotide
cctcacgcagagctccatc

SEQ ID NO: 161 - DNA, 19 bp, Artificial Sequence
  Description of Artificial Sequence Synthetic oligonucleotide
catggtgtacttcaacttc

SEQ ID NO: 162 - DNA, 19 bp, Artificial Sequence
  Description of Artificial Sequence Synthetic oligonucleotide
gtgtacttcaacttctttg

SEQ ID NO: 163 - DNA, 19 bp, Artificial Sequence
  Description of Artificial Sequence Synthetic oligonucleotide
aatccggaaccagatcata

SEQ ID NO: 164 - DNA, 19 bp, Artificial Sequence
  Description of Artificial Sequence Synthetic oligonucleotide
gtacatcgtgggcttctat

SEQ ID NO: 165 - DNA, 19 bp, Artificial Sequence
  Description of Artificial Sequence Synthetic oligonucleotide
caagccctccaacatccta

SEQ ID NO: 166 - DNA, 19 bp, Artificial Sequence
  Description of Artificial Sequence Synthetic oligonucleotide
tcgactccatggccaactc

SEQ ID NO: 167 - DNA, 19 bp, Artificial Sequence
  Description of Artificial Sequence Synthetic oligonucleotide
catggccaactccttcgtg

SEQ ID NO: 168 - DNA, 19 bp, Artificial Sequence
  Description of Artificial Sequence Synthetic oligonucleotide
tgagcaatgaccatatctg

SEQ ID NO: 169 - DNA, 19 bp, Artificial Sequence
  Description of Artificial Sequence Synthetic oligonucleotide
ccaagggctataccaagtc

SEQ ID NO: 170 - DNA, 19 bp, Artificial Sequence
  Description of Artificial Sequence Synthetic oligonucleotide
gtctgtgggctgcattctg

SEQ ID NO: 171 - DNA, 19 bp, Artificial Sequence
  Description of Artificial Sequence Synthetic oligonucleotide
ggaggacctgaattgtatc

SEQ ID NO: 172 - DNA, 19 bp, Artificial Sequence
  Description of Artificial Sequence Synthetic oligonucleotide
ccggcaggagctgaacaag

SEQ ID NO: 173 - DNA, 19 bp, Artificial Sequence
  Description of Artificial Sequence Synthetic oligonucleotide
aactggatgcattacaacc

SEQ ID NO: 174 - DNA, 19 bp, Artificial Sequence
  Description of Artificial Sequence Synthetic oligonucleotide
caactggatgcattacaac

SEQ ID NO: 175 - DNA, 19 bp, Artificial Sequence
  Description of Artificial Sequence Synthetic oligonucleotide
gaccgagtcttgctctgtt

SEQ ID NO: 176 - DNA, 19 bp, Artificial Sequence
  Description of Artificial Sequence Synthetic oligonucleotide
tacccaggctggagtgcag

SEQ ID NO: 177 - DNA, 19 bp, Artificial Sequence
  Description of Artificial Sequence Synthetic oligonucleotide
gacctacatctccaagaag

SEQ ID NO: 178 - DNA, 19 bp, Artificial Sequence
  Description of Artificial Sequence Synthetic oligonucleotide
agaatgtgctggtcatctg

SEQ ID NO: 179 - DNA, 19 bp, Artificial Sequence
  Description of Artificial Sequence Synthetic oligonucleotide
gcaccttcatggacggcta

SEQ ID NO: 180 - DNA, 19 bp, Artificial Sequence
  Description of Artificial Sequence Synthetic oligonucleotide
gcattatggtgtccgcaca

SEQ ID NO: 181 - DNA, 19 bp, Artificial Sequence
  Description of Artificial Sequence Synthetic oligonucleotide
tgaaggaagttgtccgaac

SEQ ID NO: 182 - DNA, 19 bp, Artificial Sequence
  Description of Artificial Sequence Synthetic oligonucleotide
ctgcagaaccgcatcgcca

SEQ ID NO: 183 - DNA, 19 bp, Artificial Sequence
  Description of Artificial Sequence Synthetic oligonucleotide
ccacgtgactttcaacaat

SEQ ID NO: 184 - DNA, 19 bp, Artificial Sequence
  Description of Artificial Sequence Synthetic oligonucleotide
acattcagatgcacaacta

SEQ ID NO: 185 - DNA, 19 bp, Artificial Sequence
  Description of Artificial Sequence Synthetic oligonucleotide
ggacaagcgtcgccggaat

SEQ ID NO: 186 - DNA, 19 bp, Artificial Sequence
  Description of Artificial Sequence Synthetic oligonucleotide
ccaccttgcttgtaccaaa

SEQ ID NO: 187 - RNA, 19 nt, Artificial Sequence
  Description of Artificial Sequence Synthetic oligonucleotide
gacaaggacgggcacauua

SEQ ID NO: 188 - RNA, 19 nt, Artificial Sequence
  Description of Artificial Sequence Synthetic oligonucleotide
gcuacuuccuccucaagaa

SEQ ID NO: 189 - RNA, 19 nt, Artificial Sequence
  Description of Artificial Sequence Synthetic oligonucleotide
gaccgccucugcuuuguca

SEQ ID NO: 190 - RNA, 19 nt, Artificial Sequence
  Description of Artificial Sequence Synthetic oligonucleotide
ggcagcacguguacgagaa

SEQ ID NO: 191 - RNA, 19 nt, Artificial Sequence
  Description of Artificial Sequence Synthetic oligonucleotide
guacagcgcccacgcauuc

SEQ ID NO: 192 - RNA, 19 nt, Artificial Sequence
  Description of Artificial Sequence Synthetic oligonucleotide
gaaacagaacgcggcucua

SEQ ID NO: 193 - RNA, 19 nt, Artificial Sequence
  Description of Artificial Sequence Synthetic oligonucleotide
gaacgcggcucuacgcaag

SEQ ID NO: 194 - RNA, 19 nt, Artificial Sequence
  Description of Artificial Sequence Synthetic oligonucleotide
agaguucagaggagggaga

SEQ ID NO: 195 - DNA, 37 bp, Artificial Sequence
  Description of Artificial Sequence Synthetic oligonucleotide
taatacgactcactatagcgactggttacccggtcgt

SEQ ID NO: 196 - DNA, 28 bp, Artificial Sequence
  Description of Artificial Sequence Synthetic primer
taatacgactcactatagcgactggtta

SEQ ID NO: 197 - DNA, 20 bp, Artificial Sequence
  Description of Artificial Sequence Synthetic primer
acgaccgggtaaccagtcgc

SEQ ID NO: 198 - DNA, 64 bp, Artificial Sequence
  Description of Artificial Sequence Synthetic oligonucleotide
gtaatacgactcactataggcgactggttacccggtcgcaattggccaagatccaagataaatt

SEQ ID NO: 199 - DNA, 64 bp, Artificial Sequence
  Description of Artificial Sequence Synthetic oligonucleotide
  Description of Combined DNA/RNA Molecule Synthetic oligonucleotide
aatttatcttggaucttggccaattgcgaccgggtaaccagtcgcctatagtgagtcgtattac

SEQ ID NO: 200 - DNA, 64 bp, Artificial Sequence
  Description of Artificial Sequence Synthetic oligonucleotide
gtaatacgactcactataggcgactggttacccggtcgcaaaatttatcttggatcttggcctt

SEQ ID NO: 201 - DNA, 64 bp, Artificial Sequence
  Description of Artificial Sequence Synthetic oligonucleotide
aaggccaagatccaagataaattttgcgaccgggtaaccagtcgcctatagtgagtcgtattac

SEQ ID NO: 202 - RNA, 89 nt, Artificial Sequence
  Description of Artificial Sequence Synthetic oligonucleotide
gggagacaaacaaagagcgacaagggcagauagcuuuuaguugugcaaugcucugcaccgucgaguucccacccagaagaagccagaag

SEQ ID NO: 203 - RNA, 89 nt, Artificial Sequence
  Description of Artificial Sequence Synthetic oligonucleotide
gggagacaaacaaagagcgacaagggcagugagauaguagacgaggagguuccauuagaaugcaaauaucacccagaagaagccagaag

SEQ ID NO: 204 - RNA, 88 nt, Artificial Sequence
  Description of Artificial Sequence Synthetic oligonucleotide
gggagacaaacaaagagcgacaagggcagagguaccaagcagagggucuaaggguagcccggacgagucacccagaagaagccagaag

SEQ ID NO: 205 - RNA, 89 nt, Artificial Sequence
  Description of Artificial Sequence Synthetic oligonucleotide
gggagacaaacaaagagcgacaagggcagcaucugcuaguaauguucggcggucgaacucuacuuggaacacccagaagaagccagaag

SEQ ID NO: 206 - RNA, 89 nt, Artificial Sequence
  Description of Artificial Sequence Synthetic oligonucleotide
gggagacaaacaaagagcgacaagggcagagacagguuaggggaaaguguguuaaacuuuaaaguaauucacccagaagaagccagaag

SEQ ID NO: 207 - RNA, 89 nt, Artificial Sequence
  Description of Artificial Sequence Synthetic oligonucleotide
gggagacaaacaaagagcgacaagggcagguaggacgucaguuuaacgcaagcgcguuacaccuagauccacccagaagaagccagaag

SEQ ID NO: 208 - RNA, 89 nt, Artificial Sequence
  Description of Artificial Sequence Synthetic oligonucleotide
gggagacaaacaaagagcgacaagggcagccuauuuuggggcugugacauauugucaaaugcuaaacggcacccagaagaagccagaag

SEQ ID NO: 209 - RNA, 89 nt, Artificial Sequence
  Description of Artificial Sequence Synthetic oligonucleotide
gggagacaaacaaagagcgacaagggcagagcaguuucguugggcgucggucuaauagacugacuggggcacccagaagaagccagaag

SEQ ID NO: 210 - RNA, 89 nt, Artificial Sequence
  Description of Artificial Sequence Synthetic oligonucleotide
gggagacaaacaaagagcgacaagggcagguccaaucugagugaugucugucaagaccuagagaaguaccacccagaagaagccagaag

SEQ ID NO: 211 - RNA, 89 nt, Artificial Sequence
  Description of Artificial Sequence Synthetic oligonucleotide
gggagacaaacaaagagcgacaagggcagagugaugucugucaagaccgggcucuaccgcugguucaagcacccagaagaagccagaag

SEQ ID NO: 212 - RNA, 89 nt, Artificial Sequence
  Description of Artificial Sequence Synthetic oligonucleotide
gggagacaaacaaagagcgacaagggcagugggagcacuagaauggucuauauuaauguuagcgcagugcacccagaagaagccagaag

SEQ ID NO: 213 - RNA, 51 nt, Artificial Sequence
  Description of Artificial Sequence Synthetic oligonucleotide
aggagaugcguaggguggacugagugaugucugucaagaccucguccaaac

SEQ ID NO: 214 - RNA, 51 nt, Artificial Sequence
  Description of Artificial Sequence Synthetic oligonucleotide
aggagugaugucugucaagaccgauugucugccacucaaucgggaccaaac

SEQ ID NO: 215 - RNA, 51 nt, Artificial Sequence
  Description of Artificial Sequence Synthetic oligonucleotide
aggagugaugucugucaagaccaggugcugaggugacucuguaauucaaac

SEQ ID NO: 216 - RNA, 40 nt, Artificial Sequence
  Description of Artificial Sequence Synthetic oligonucleotide
gggaggacgaugcggaucagccauguuuacgucacuccua

SEQ ID NO: 217 - RNA, 67 nt, Artificial Sequence
  Description of Artificial Sequence Synthetic oligonucleotide
gcccagcaugcauuacugaucgugguguuugcuuagcccaaaggccaagauccaagauaaagaaggc

SEQ ID NO: 218 - RNA, 69 nt, Artificial Sequence
  Description of Artificial Sequence Synthetic oligonucleotide
gcccagcaugcauuacugaucgugguguuugcuuagcccaaagccuucuuuaucuuggaucuuggccuu

SEQ ID NO: 219 - RNA, 40 nt, Artificial Sequence
  Description of Artificial Sequence Synthetic oligonucleotide
gcccagcaugcauuacugaucgugguguuugcuuagccca

SEQ ID NO: 220 - RNA, 41 nt, Artificial Sequence
  Description of Artificial Sequence Synthetic oligonucleotide
ggcuggggugcgacauuauguucguuaaggaucaaucgcuc

SEQ ID NO: 221 - RNA, 40 nt, Artificial Sequence
  Description of Artificial Sequence Synthetic oligonucleotide
uuaugaugcaaaaacgaacuggaauggccaugcagguaca

SEQ ID NO: 222 - RNA, 41 nt, Artificial Sequence
  Description of Artificial Sequence Synthetic oligonucleotide
gguuggggugcgacauuauguucguuaaggaucaaucgcuc

SEQ ID NO: 223 - RNA, 41 nt, Artificial Sequence
  Description of Artificial Sequence Synthetic oligonucleotide
gauuuggagagcauuauguuagguuaaggaucaaucuucua

SEQ ID NO: 224 - RNA, 41 nt, Artificial Sequence
  Description of Artificial Sequence Synthetic oligonucleotide
ggcuggggugcgacauuauguucguuaaggaucaaucgcuu

SEQ ID NO: 225 - RNA, 83 nt, Artificial Sequence
  Description of Artificial Sequence Synthetic oligonucleotide
gggguaaacgaacaggauuuuuuuaacgagcuauauuguuuccuguugcccguccgucuagucgugaagagagcaagguuacu

SEQ ID NO: 226 - RNA, 83 nt, Artificial Sequence
  Description of Artificial Sequence Synthetic oligonucleotide
gggguaaacgaacaggacgacgggucgaagcugaauagguaaccaaucacggcauaacuagucgugaagagagcaagguuacu

SEQ ID NO: 227 - RNA, 83 nt, Artificial Sequence
  Description of Artificial Sequence Synthetic oligonucleotide
gggguaaacgaacaggaugagggagcaaaaagggcgaaaaugcaguaacuaaacguucuagucgugaagagagcaagguuacu

SEQ ID NO: 228 - RNA, 83 nt, Artificial Sequence
  Description of Artificial Sequence Synthetic oligonucleotide
gggguaaacgaacaggauuuuuuuaacgagcuauauuauuuccuguugcccguccgucuagucgugaagagagcaagguuacu

SEQ ID NO: 229 - RNA, 84 nt, Artificial Sequence
  Description of Artificial Sequence Synthetic oligonucleotide
gggguaaacgaacaggaaccauuaaaucauaaggagaaagaugaugugcgcgacauaacuagucgugaagagagcaagguuacu

SEQ ID NO: 230 - RNA, 89 nt, Artificial Sequence
  Description of Artificial Sequence Synthetic oligonucleotide
gggagacaaacaaagagcgacaagggcagcggguuaucacguugggaacgggccaucaacucuucucaccacccagaagaagccagaag

SEQ ID NO: 231 - RNA, 89 nt, Artificial Sequence
  Description of Artificial Sequence Synthetic oligonucleotide
gggagacaaacaaagagcgacaagggcaguagugagggauucaccagagugaaugcgcuccucggaaaucacccagaagaagccagaag

SEQ ID NO: 232 - RNA, 89 nt, Artificial Sequence
  Description of Artificial Sequence Synthetic oligonucleotide
gggagacaaacaaagagcgacaagggcagaacgggcaauguccaaggugaggcaguuuguauggacacacacccagaagaagccagaag

SEQ ID NO: 233 - RNA, 89 nt, Artificial Sequence
  Description of Artificial Sequence Synthetic oligonucleotide
gggagacaaacaaagagcgacaagggcaguugagauugaggagucagaccugcgucucuaguaacaaugcacccagaagaagccagaag

SEQ ID NO: 234 - RNA, 89 nt, Artificial Sequence
  Description of Artificial Sequence Synthetic oligonucleotide
gggagacaaacaaagagcgacaagggcagaguggacggucggcuagagccgggaggaauuccuugugaccacccagaagaagccagaag

SEQ ID NO: 235 - RNA, 89 nt, Artificial Sequence
  Description of Artificial Sequence Synthetic oligonucleotide
gggagacaaacaaagagcgacaagggcaguuugacaauguaccuuuaauuacggauuguaccuugggcgcacccagaagaagccagaag

SEQ ID NO: 236 - RNA, 35 nt, Artificial Sequence
  Description of Artificial Sequence Synthetic oligonucleotide
gggagagaggaagagggaugggccgacgugccgca

SEQ ID NO: 237 - RNA, 89 nt, Artificial Sequence
  Description of Artificial Sequence Synthetic oligonucleotide
gggagacaaacaaagagcgacaagggcagaauuacaauagcuauaguccgggcaccaugcuuguaaauccacccagaagaagccagaag

SEQ ID NO: 238 - RNA, 89 nt, Artificial Sequence
  Description of Artificial Sequence Synthetic oligonucleotide
gggagacaaacaaagagcgacaagggcagguaggacgcuagcagacuagaauguaucuaugcuuagauccacccagaagaagccagaag

SEQ ID NO: 239 - RNA, 89 nt, Artificial Sequence
  Description of Artificial Sequence Synthetic oligonucleotide
gggagacaaacaaagagcgacaagggcaggcuaguauuacaaugucguggaaaagccgugcgggguauccacccagaagaagccagaag

SEQ ID NO: 240 - RNA, 89 nt, Artificial Sequence
  Description of Artificial Sequence Synthetic oligonucleotide
gggagacaaacaaagagcgacaagggcagggagccauucuugaaauugucaguuugauugugcucaggucacccagaagaagccagaag

SEQ ID NO: 241 - RNA, 89 nt, Artificial Sequence
  Description of Artificial Sequence Synthetic oligonucleotide
gggagacaaacaaagagcgacaagggcagaaaguacaaugguugacauauaccgucgguuuauccuaugcacccagaagaagccagaag

SEQ ID NO: 242 - RNA, 89 nt, Artificial Sequence
  Description of Artificial Sequence Synthetic oligonucleotide
gggagacaaacaaagagcgacaagggcagguaggcuaucgcugcuugaucgucugaucagagccuauaccacccagaagaagccagaag

SEQ ID NO: 243 - RNA, 83 nt, Artificial Sequence
  Description of Artificial Sequence Synthetic oligonucleotide
gggguaaacgaacaggaaacagauggccaacacaggcgaagcauagacuaggaacggcuagucgugaagagagcaagguuacu

SEQ ID NO: 244 - RNA, 81 nt, Artificial Sequence
  Description of Artificial Sequence Synthetic oligonucleotide
gggguaaacgaacaggacuugaugugaaaaggcgacgcgaugagacgaagggcuucuagucgugaagagagcaagguuacu

SEQ ID NO: 245 - RNA, 83 nt, Artificial Sequence
  Description of Artificial Sequence Synthetic oligonucleotide
gggguaaacgaacaggaaguagacuagacggcggcgauaaccagauaacgacauucucuagucgugaagagagcaagguuacu

SEQ ID NO: 246 - RNA, 83 nt, Artificial Sequence
  Description of Artificial Sequence Synthetic oligonucleotide
gggguaaacgaacaggaccgagugagacggguaguggacaaaugaaguagugugguccuagucgugaagagagcaagguuacu

SEQ ID NO: 247 - RNA, 83 nt, Artificial Sequence
  Description of Artificial Sequence Synthetic oligonucleotide
gggguaaacgaacaggacuuuuaauuucacgccgcacgauccggaaaaacgacuugacuagucgugaagagagcaagguuacu

SEQ ID NO: 248 - DNA, 21 bp, Unknown
  Description of Unknown target sequence
ctgattaatatatttaatata

SEQ ID NO: 249 - DNA, 21 bp, Unknown
  Description of Unknown target sequence
ctccttccacatgtacataaa

SEQ ID NO: 250 - DNA, 21 bp, Unknown
  Description of Unknown target sequence
cagcattatggtgtccgcaca

SEQ ID NO: 251 - DNA, 21 bp, Unknown
  Description of Unknown target sequence
cagcaccttcatggacggcta

SEQ ID NO: 252 - DNA, 19 bp, Unknown
  Description of Unknown target sequence
gcaccttcatggacggcta

SEQ ID NO: 253 - DNA, 18 bp, Unknown
  Description of Unknown target sequence
gcatatggtgtccgcaca

SEQ ID NO: 254 - DNA, 18 bp, Unknown
  Description of Unknown target sequence
tgaaggaagtgtccgaac

SEQ ID NO: 255 - DNA, 19 bp, Unknown
  Description of Unknown target sequence
ctgcagaaccgcatcgcca

SEQ ID NO: 256 - DNA, 19 bp, Unknown
  Description of Unknown target sequence
tgctgtgtgtggggacaac

SEQ ID NO: 257 - DNA, 19 bp, Unknown
  Description of Unknown target sequence
gggctgcaagggcttcttc

SEQ ID NO: 258 - DNA, 19 bp, Unknown
  Description of Unknown target sequence
gcgcacagtgcagaaaaac

SEQ ID NO: 259 - DNA, 17 bp, Unknown
  Description of Unknown target sequence
cagtggctctgactact

SEQ ID NO: 260 - DNA, 19 bp, Unknown
  Description of Unknown target sequence
ccacttctccacaccttga

SEQ ID NO: 261 - DNA, 18 bp, Unknown
  Description of Unknown target sequence
ggcttgagctgcagaatg

SEQ ID NO: 262 - DNA, 21 bp, Unknown
  Description of Unknown target sequence
cacagcttgcttgtcgatgtc

SEQ ID NO: 263 - DNA, 25 bp, Unknown
  Description of Unknown target sequence
ggtccctgcacagcttgcttgtcga

SEQ ID NO: 264 - DNA, 25 bp, Unknown
  Description of Unknown target sequence
ccggttctctggaggtcatccgcaa

SEQ ID NO: 265 - DNA, 25 bp, Unknown
  Description of Unknown target sequence
cagcattatggtgtccgcacatgtg

SEQ ID NO: 266 - DNA, 21 bp, Unknown
  Description of Unknown target sequence
aatgttgacggacagcctatt

SEQ ID NO: 267 - DNA, 21 bp, Unknown
  Description of Unknown target sequence
aagagtacggccctgaagaag

SEQ ID NO: 268 - DNA, 21 bp, Unknown
  Description of Unknown target sequence
aaggaggtttgtataagtaat

SEQ ID NO: 269 - DNA, 21 bp, Unknown
  Description of Unknown target sequence
caggagcgcattgcacatcaa

SEQ ID NO: 270 - DNA, 21 bp, Unknown
  Description of Unknown target sequence
ccctattagatacacttctta

SEQ ID NO: 271 - DNA, 21 bp, Unknown
  Description of Unknown target sequence
taaggaggtttgtataagtaa

SEQ ID NO: 272 - DNA, 21 bp, Unknown
  Description of Unknown target sequence
cctagtcaagcctgagaatta

SEQ ID NO: 273 - DNA, 19 bp, Unknown
  Description of Unknown target sequence
ctgccagtgtatactctga

SEQ ID NO: 274 - DNA, 19 bp, Unknown
  Description of Unknown target sequence
atacactcggtagctgtgg

SEQ ID NO: 275 - RNA, 41 nt, Artificial Sequence
  Description of Artificial Sequence Synthetic oligonucleotide
uauacauucuugguucauaaaggauaaggccuaagucgggu

SEQ ID NO: 276 - RNA, 51 nt, Artificial Sequence
  Description of Artificial Sequence Synthetic oligonucleotide
aggaguauacauucuugguucauaaaggauaaggccuaagucgggucaaac

SEQ ID NO: 277 - RNA, 87 nt, Artificial Sequence
  Description of Artificial Sequence Synthetic oligonucleotide
gggagacaagaauaaacgcucaagacacggauacauaaugcugucuugauuuacaaacugagcuucgacaggaggcucacaacaggc

SEQ ID NO: 278 - RNA, 87 nt, Artificial Sequence
  Description of Artificial Sequence Synthetic oligonucleotide
gggagacaagaauaaacgcucaaugagcuuacagcggccauugauuuacuaacggacugagcauucgacaggaggcucacaacaggc

SEQ ID NO: 279 - RNA, 79 nt, Artificial Sequence
  Description of Artificial Sequence Synthetic oligonucleotide
gaauuccgcgugugccagcgaaaguugcguaugggucacaucgcaggcacaugucaucugggcgguccguucgggaucc

SEQ ID NO: 280 - RNA, 51 nt, Artificial Sequence
  Description of Artificial Sequence Synthetic oligonucleotide
aggaggguugcguugcaaguaacagaaaggaauuugaaaauuguggcaaac

SEQ ID NO: 281 - RNA, 51 nt, Artificial Sequence
  Description of Artificial Sequence Synthetic oligonucleotide
aggagguuggcaaucccggauugaggaaucgcaugacgcuauuaaccaaac

SEQ ID NO: 282 - RNA, 70 nt, Artificial Sequence
  Description of Artificial Sequence Synthetic oligonucleotide
gggagagagauauaagggccuccugauacccgcugcuaucuggaccgaucccauuaccaaauucucuccc

SEQ ID NO: 283 - RNA, 83 nt, Artificial Sequence
  Description of Artificial Sequence Synthetic oligonucleotide
gggguaaacgaacaggaagacggcgcaauaagacagacuaggacacgauuagagguacuagucgugaagagagcaagguuacu

SEQ ID NO: 284 - RNA, 83 nt, Artificial Sequence
  Description of Artificial Sequence Synthetic oligonucleotide
gggguaaacgaacaggauaaaagaaaacaacuagcgcgacgagagaauaaaaugaaacuagucgugaagagagcaagguuacu

SEQ ID NO: 285 - RNA, 83 nt, Artificial Sequence
  Description of Artificial Sequence Synthetic oligonucleotide
gggguaaacgaacaggauaauuguuggggaaauaaauugcugggaacgacuuaaaagcuagucgugaagagagcaagguuacu

SEQ ID NO: 286 - RNA, 83 nt, Artificial Sequence
  Description of Artificial Sequence Synthetic oligonucleotide
gggguaaacgaacaggaguuaaucaugagguagguaacaaaaggcaacggccaauaacuagucgugaagagagcaagguuacu

SEQ ID NO: 287 - RNA, 83 nt, Artificial Sequence
  Description of Artificial Sequence Synthetic oligonucleotide
gggguaaacgaacaggauaaccaugcaaauaacaagcaaacagagaacucacgccagcuagucgugaagagagcaagguuacu

SEQ ID NO: 288 - RNA, 41 nt, Artificial Sequence
  Description of Artificial Sequence Synthetic oligonucleotide
uaguaaaugagagaugaaaucuguaugcgccgcacugauug

SEQ ID NO: 289 - RNA, 51 nt, Artificial Sequence
  Description of Artificial Sequence Synthetic oligonucleotide
aggaguagugcagcauugacugaaugucauacggcauaagcaucuacaaac

SEQ ID NO: 290 - RNA, 51 nt, Artificial Sequence
  Description of Artificial Sequence Synthetic oligonucleotide
aggaggacaucggaaacgcugaucuuaauagugaauuaacaugcgacaaac

SEQ ID NO: 291 - RNA, 51 nt, Artificial Sequence
  Description of Artificial Sequence Synthetic oligonucleotide
aggaguaguaaaugagagaugaaaucuguaugcgccgcacugauugcaaac

SEQ ID NO: 292 - RNA, 89 nt, Artificial Sequence
  Description of Artificial Sequence Synthetic oligonucleotide
gggagacaaacaaagagcgacaagggcagggaugaccgguguauuaaggucuaacuuaacucggugauacacccagaagaagccagaag

SEQ ID NO: 293 - RNA, 89 nt, Artificial Sequence
  Description of Artificial Sequence Synthetic oligonucleotide
gggagacaaacaaagagcgacaagggcagcucaaaaaggguagugugugguauagucuaaucguacccacacccagaagaagccagaag

SEQ ID NO: 294 - RNA, 89 nt, Artificial Sequence
  Description of Artificial Sequence Synthetic oligonucleotide
gggagacaaacaaagagcgacaagggcaguagugugguauuguguaauaauacccuacugaggucaaaacacccagaagaagccagaag

SEQ ID NO: 295 - RNA, 89 nt, Artificial Sequence
  Description of Artificial Sequence Synthetic oligonucleotide
gggagacaaacaaagagcgacaagggcagcggguugucaagaugggaacgggcccggaucuuuagcgcacacccagaagaagccagaag

SEQ ID NO: 296 - RNA, 89 nt, Artificial Sequence
  Description of Artificial Sequence Synthetic oligonucleotide
gggagacaaacaaagagcgacaagggcagugggcugugcgcgcgaugagaucacguuagcguaauugugcacccagaagaagccagaag

SEQ ID NO: 297 - RNA, 89 nt, Artificial Sequence
  Description of Artificial Sequence Synthetic oligonucleotide
gggagacaaacaaagagcgacaagggcagacagguccaucaggcagaaccgagggagagugcgcgucgucacccagaagaagccagaag

SEQ ID NO: 298 - RNA, 89 nt, Artificial Sequence
  Description of Artificial Sequence Synthetic oligonucleotide
gggagacaaacaaagagcgacaagggcagaaguuuggauuucaagaugcucaucacgcucaaacuuucacacccagaagaagccagaag

SEQ ID NO: 299 - RNA, 89 nt, Artificial Sequence
  Description of Artificial Sequence Synthetic oligonucleotide
gggagacaaacaaagagcgacaagggcaggacgcugcgaaaagugcgaaguuugcauccuggccuaguucacccagaagaagccagaag

SEQ ID NO: 300 - RNA, 89 nt, Artificial Sequence
  Description of Artificial Sequence Synthetic oligonucleotide
gggagacaaacaaagagcgacaagggcaguucucuccacaaaguuuagauuucaagcgugagcagggaucacccagaagaagccagaag

SEQ ID NO: 301 - RNA, 89 nt, Artificial Sequence
  Description of Artificial Sequence Synthetic oligonucleotide
gggagacaaacaaagagcgacaagggcagguguugagaugaaggaguucuagcccuucgaauggugugacacccagaagaagccagaag

SEQ ID NO: 302 - RNA, 89 nt, Artificial Sequence
  Description of Artificial Sequence Synthetic oligonucleotide
gggagacaaacaaagagcgacaagggcagaguuggguagguugugacaggaaugugauugguaagauaccacccagaagaagccagaag

SEQ ID NO: 303 - RNA, 89 nt, Artificial Sequence
  Description of Artificial Sequence Synthetic oligonucleotide
gggagacaaacaaagagcgacaagggcaguacuugagucauuguauagaucuaauucgcgcagaauugacacccagaagaagccagaag

SEQ ID NO: 304 - RNA, 92 nt, Artificial Sequence
  Description of Artificial Sequence Synthetic oligonucleotide
gggagacaaacaaagagcgacaagggcagcccuugucgcucuuuguuugucucccuauagugagucguauuacacccagaagaagccagaag

SEQ ID NO: 305 - RNA, 89 nt, Artificial Sequence
  Description of Artificial Sequence Synthetic oligonucleotide
gggagacaaacaaagagcgacaagggcagguaggacgcuagcagacuagaaugaaucuaugcuuagauccacccagaagaagccagaag

SEQ ID NO: 306 - RNA, 89 nt, Artificial Sequence
  Description of Artificial Sequence Synthetic oligonucleotide
gggagacaaacaaagagcgacaagggcagcgguuaaaauauaguucuaaguuagucuggugaauccacucacccagaagaagccagaag

SEQ ID NO: 307 - RNA, 89 nt, Artificial Sequence
  Description of Artificial Sequence Synthetic oligonucleotide
gggagacaaacaaagagcgacaagggcagucugggggauggcugugaggucccaauguaucgcaucucacacccagaagaagccagaag

SEQ ID NO: 308 - RNA, 86 nt, Artificial Sequence
  Description of Artificial Sequence Synthetic oligonucleotide
gggagacaaacaaagagcgacaagggcaguuaaaauauaguucuaaguuagucuggugaauccacucacccagaagaagccagaag

SEQ ID NO: 309 - RNA, 83 nt, Artificial Sequence
  Description of Artificial Sequence Synthetic oligonucleotide
gggguaaacgaacaggaagggagucgauuugaguuguaauuugaccuauguuauaaucuagucgugaagagagcaagguuacu

SEQ ID NO: 310 - RNA, 83 nt, Artificial Sequence
  Description of Artificial Sequence Synthetic oligonucleotide
gggguaaacgaacaggaauggcuacaguaucgaugcaguuuucgaaugaaguagaaacuagucgugaagagagcaagguuacu

SEQ ID NO: 311 - RNA, 83 nt, Artificial Sequence
  Description of Artificial Sequence Synthetic oligonucleotide
gggguaaacgaacaggacaggacagcaagcaguagaaaacaagccacgaaggggacucuagucgugaagagagcaagguuacu

SEQ ID NO: 312 - RNA, 83 nt, Artificial Sequence
  Description of Artificial Sequence Synthetic oligonucleotide
gggguaaacgaacaggauuuuggacugucuagccgauguacuuaaguuuaucauuuucuagucgugaagagagcaagguuacu

SEQ ID NO: 313 - RNA, 83 nt, Artificial Sequence
  Description of Artificial Sequence Synthetic oligonucleotide
gggguaaacgaacaggagcagucgcuggcuucauuuuuuuuuuuuuuuugugcucaacuagucgugaagagagcaagguuacu

SEQ ID NO: 314 - RNA, 51 nt, Artificial Sequence
  Description of Artificial Sequence Synthetic oligonucleotide
aggagacacaugugacaagaggcuaugauccugaaugcauccuuggcaaac

SEQ ID NO: 315 - RNA, 51 nt, Artificial Sequence
  Description of Artificial Sequence Synthetic oligonucleotide
aggagaaucaugaguuaucuguguaaggaaccaaagccaugcuuaucaaac

SEQ ID NO: 316 - RNA, 19 nt, Artificial Sequence
  Description of Artificial Sequence Synthetic oligonucleotide
aggccaagauccaagauaa

SEQ ID NO: 317 - RNA, 19 nt, Artificial Sequence
  Description of Artificial Sequence Synthetic oligonucleotide
ggccaagauccaggauaaa

SEQ ID NO: 318 - RNA, 19 nt, Artificial Sequence
  Description of Artificial Sequence Synthetic oligonucleotide
ggccaagauccaggauaag

SEQ ID NO: 319 - RNA, 19 nt, Artificial Sequence
  Description of Artificial Sequence Synthetic oligonucleotide
ggccaagauccaagauaaa

SEQ ID NO: 320 - RNA, 19 nt, Artificial Sequence
  Description of Artificial Sequence Synthetic oligonucleotide
ggcaaagauccaagauaag

SEQ ID NO: 321 - RNA, 19 nt, Artificial Sequence
  Description of Artificial Sequence Synthetic oligonucleotide
ugcagaucuucgugaagac

SEQ ID NO: 322 - RNA, 19 nt, Artificial Sequence
  Description of Artificial Sequence Synthetic oligonucleotide
agcccagugacaccaucga

SEQ ID NO: 323 - RNA, 19 nt, Artificial Sequence
  Description of Artificial Sequence Synthetic oligonucleotide
gacuacaacauccagaaag

SEQ ID NO: 324 - RNA, 19 nt, Artificial Sequence
  Description of Artificial Sequence Synthetic oligonucleotide
cuacaacauccagaaagag

SEQ ID NO: 325 - RNA, 19 nt, Artificial Sequence
  Description of Artificial Sequence Synthetic oligonucleotide
agugacaccaucgaaaaug

SEQ ID NO: 326 - DNA, 933 bp, Homo sapiens
atttaggggcggttggctttgttgggtgagcttgtttgtgtccctgtgggtggacgtggttggtgattggcaggatcctggtatccgctaacaggtcaaaatgcagatcttcgtgaaaacccttaccggcaagaccatcacccttgaggtggagcccagtgacaccatcgaaaatgtgaaggccaagatccaggataaggaaggcattccccccgaccagcagaggctcatctttgcaggcaagcagctggaagatggccgtactctttctgactacaacatccagaaggagtcgaccctgcacctggtcctgcgtctgagaggtggtatgcagatcttcgtgaagaccctgaccggcaagaccatcaccctggaagtggagcccagtgacaccatcgaaaatgtgaaggccaagatccaggataaagaaggcatccctcccgaccagcagaggctcatctttgcaggcaagcagctggaagatggccgcactctttctgactacaacatccagaaggagtcgaccctgcacctggtcctgcgtctgagaggtggtatgcagatcttcgtgaagaccctgaccggcaagaccatcactctggaggtggagcccagtgacaccatcgaaaatgtgaaggccaagatccaagataaagaaggcatcccccccgaccagcagaggctcatctttgcaggcaagcagctggaagatggccgcactctttctgactacaacatccagaaagagtcgaccctgcacctggtcctgcgcctgaggggtggctgttaattcttcagtcatggcattcgcagtgcccagtgatggcattactctgcactatagccatttgccccaacttaagtttagaaattacaagtttcagtaatagctgaacctgttcaaaatgttaataaaggtttcgttgcatggta

SEQ ID NO: 327 - DNA, 2193 bp, Homo sapiens
agttccgtcgcagccgggatttgggtcgcagttcttgtttgtggatcgctgtgatcgtcacttgacaatgcagatcttcgtgaagactctgactggtaagaccatcaccctcgaggttgagcccagtgacaccatcgagaatgtcaaggcaaagatccaagataaggaaggcatccctcctgaccagcagaggctgatctttgctggaaaacagctggaagatgggcgcaccctgtctgactacaacatccagaaagagtccaccctgcacctggtgctccgtctcagaggtgggatgcaaatcttcgtgaagacactcactggcaagaccatcacccttgaggtcgagcccagtgacaccatcgagaacgtcaaagcaaagatccaggacaaggaaggcattcctcctgaccagcagaggttgatctttgccggaaagcagctggaagatgggcgcaccctgtctgactacaacatccagaaagagtctaccctgcacctggtgctccgtctcagaggtgggatgcagatcttcgtgaagaccctgactggtaagaccatcaccctcgaggtggagcccagtgacaccatcgagaatgtcaaggcaaagatccaagataaggaaggcattcctcctgatcagcagaggttgatctttgccggaaaacagctggaagatggtcgtaccctgtctgactacaacatccagaaagagtccaccttgcacctggtactccgtctcagaggtgggatgcaaatcttcgtgaagacactcactggcaagaccatcacccttgaggtcgagcccagtgacactatcgagaacgtcaaagcaaagatccaagacaaggaaggcattcctcctgaccagcagaggttgatctttgccggaaagcagctggaagatgggcgcaccctgtctgactacaacatccagaaagagtctaccctgcacctggtgctccgtctcagaggtgggatgcagatcttcgtgaagaccctgactggtaagaccatcactctcgaagtggagccgagtgacaccattgagaatgtcaaggcaaagatccaagacaaggaaggcatccctcctgaccagcagaggttgatctttgccggaaaacagctggaagatggtcgtaccctgtctgactacaacatccagaaagagtccaccttgcacctggtgctccgtctcagaggtgggatgcagatcttcgtgaagaccctgactggtaagaccatcactctcgaggtggagccgagtgacaccattgagaatgtcaaggcaaagatccaagacaaggaaggcatccctcctgaccagcagaggttgatctttgctgggaaacagctggaagatggacgcaccctgtctgactacaacatccagaaagagtccaccctgcacctggtgctccgtcttagaggtgggatgcagatcttcgtgaagaccctgactggtaagaccatcactctcgaagtggagccgagtgacaccattgagaatgtcaaggcaaagatccaagacaaggaaggcatccctcctgaccagcagaggttgatctttgctgggaaacagctggaagatggacgcaccctgtctgactacaacatccagaaagagtccaccctgcacctggtgctccgtcttagaggtgggatgcagatcttcgtgaagaccctgactggtaagaccatcactctcgaagtggagccgagtgacaccattgagaatgtcaaggcaaagatccaagacaaggaaggcatccctcctgaccagcagaggttgatctttgctgggaaacagctggaagatggacgcaccctgtctgactacaacatccagaaagagtccaccctgcacctggtgctccgtctcagaggtgggatgcaaatcttcgtgaagaccctgactggtaagaccatcaccctcgaggtggagcccagtgacaccatcgagaatgtcaaggcaaagatccaagataaggaaggcatccctcctgatcagcagaggttgatctttgctgggaaacagctggaagatggacgcaccctgtctgactacaacatccagaaagagtccactctgcacttggtcctgcgcttgagggggggtgtctaagtttccccttttaaggtttcaacaaatttcattgcactttcctttcaataaagttgttgcattcccaa

SEQ ID NO: 328 - RNA, 19 nt, Artificial Sequence
  Description of Artificial Sequence Synthetic oligonucleotide
gcgacugguuacccggucg

SEQ ID NO: 329 - DNA, 21 bp, Artificial Sequence
  Description of Artificial Sequence Synthetic oligonucleotide
  Description of Combined DNA/RNA Molecule Synthetic oligonucleotide
ggccaagauccaagauaaatt

SEQ ID NO: 330 - DNA, 21 bp, Artificial Sequence
  Description of Artificial Sequence Synthetic oligonucleotide
  Description of Combined DNA/RNA Molecule Synthetic oligonucleotide
uuuaucuuggaucuuggcctt

SEQ ID NO: 331 - RNA, 45 nt, Artificial Sequence
  Description of Artificial Sequence Synthetic oligonucleotide
gcgacugguuacccggucguaaaaggccaagauccaagauaaauu

SEQ ID NO: 332 - RNA, 45 nt, Artificial Sequence
  Description of Artificial Sequence Synthetic oligonucleotide
gcgacugguuacccggucguaaaauuuaucuuggaucuuggccuu

SEQ ID NO: 333 - DNA, 240 bp, Homo sapiens
atcttcgtgaaaacccttaccggcaagaccatcacccttgaggtggagcccagtgacaccatcgaaaatgtgaaggccaagatccaggataaggaaggcattccccccgaccagcagaggctcatctttgcaggcaagcagctggaagatggccgtactctttctgactacaacatccagaaggagtcgaccctgcacctggtcctgcgtctgagaggtggtatgcagatcttcgtgaag

SEQ ID NO: 334 - DNA, 240 bp, Homo sapiens
atcttcgtgaagacactcactggcaagaccatcacccttgaggtcgagcccagtgacaccatcgagaacgtcaaagcaaagatccaggacaaggaaggcattcctcctgaccagcagaggttgatctttgccggaaagcagctggaagatgggcgcaccctgtctgactacaacatccagaaagagtctaccctgcacctggtgctccgtctcagaggtgggatgcagatcttcgtgaag

SEQ ID NO: 335 - DNA, 251 bp, Homo sapiens
aaggagtcgaccctgcacctggtcctgcgtctgagaggtggtatgcagatcttcgtgaagaccctgaccggcaagaccatcaccctggaagtggagcccagtgacaccatcgaaaatgtgaaggccaagatccaggataaagaaggcatccctcccgaccagcagaggctcatctttgcaggcaagcagctggaagatggccgcactctttctgactacaacatccagaaggagtcgaccctgcacctggt

SEQ ID NO: 336 - DNA, 300 bp, Homo sapiens
aaagagtctaccctgcacctggtgctccgtctcagaggtgggatgcagatcttcgtgaagaccctgactggtaagaccatcaccctcgaggtggagcccagtgacaccatcgagaatgtcaaggcaaagatccaagataaggaaggcattcctcctgatcagcagaggttgatctttgcccaagacaaggaaggcattcctcctgaccagcagaggttgatctttgccggaaagcagctggaagatgggcgcaccctgtctgactacaacatccagaaagagtctaccctgcacctggtg

SEQ ID NO: 337 - DNA, 233 bp, Homo sapiens
ggtggtatgcagatcttcgtgaagaccctgaccggcaagaccatcactctggaggtggagcccagtgacaccatcgaaaatgtgaaggccaagatccaagataaagaaggcatcccccccgaccagcagaggctcatctttgcaggcaagcagctggaagatggccgcactctttctgactacaacatccagaaagagtcgaccctgcacctggtcctgcgcctgaggggtgg

SEQ ID NO: 338 - DNA, 240 bp, Homo sapiens
ggtgggatgcagatcttcgtgaagaccctgactggtaagaccatcactctcgaggtggagccgagtgacaccattgagaatgtcaaggcaaagatccaagacaaggaaggcatccctcctgaccagcagaggttgatctttgctgggaaacagctggaagatggacgcaccctgtctgactacaacatccagaaagagtccaccctgcacctggtgctccgtcttagaggtgggatgcag

SEQ ID NO: 339 - DNA, 180 bp, Homo sapiens
ccatcactctggaggtggagcccagtgacaccatcgaaaatgtgaaggccaagatccaagataaagaaggcatcccccccgaccagcagaggctcatctttgcaggcaagcagctggaagatggccgcactctttctgactacaacatccagaaagagtcgaccctgcacctggtcctgc

SEQ ID NO: 340 - DNA, 180 bp, Mus musculus
ccatcaccttggaggtggagcccagtgacaccatcgagaatgtgaaggccaagatccaggataaagagggcatcccccctgaccagcagaggctgatctttgccggcaagcagctagaagatggccgcactctctctgattacaacatccagaaagagtcgaccctgcacctggtcctcc

SEQ ID NO: 341 - DNA, 180 bp, Homo sapiens
ccatcaccctcgaggttgagcccagtgacaccatcgagaatgtcaaggcaaagatccaagataaggaaggcatccctcctgaccagcagaggctgatctttgctggaaaacagctggaagatgggcgcaccctgtctgactacaacatccagaaagagtccaccctgcacctggtgctcc

SEQ ID NO: 342 - DNA, 180 bp, Mus musculus
ccatcaccctggaggtcgagcccagtgacaccattgagaatgtcaaggcaaagatccaggacaaggagggcatcccccctgaccagcagaggctgatctttgcaggcaagcagctggaagatggccgcaccctgtcagactacaacatccagaaagagtccaccctgcacctggtccttc

SEQ ID NO: 343 - DNA, 19 bp, Mus musculus
aggccaagatccaggataa

SEQ ID NO: 344 - DNA, 19 bp, Mus musculus
aggcaaagatccaggacaa

SEQ ID NO: 345 - RNA, 21 nt, Artificial Sequence
  Description of Artificial Sequence Synthetic oligonucleotide
aggccaagauccaagauaauu

SEQ ID NO: 346 - RNA, 21 nt, Artificial Sequence
  Description of Artificial Sequence Synthetic oligonucleotide
uuaucuuggaucuuggccuuu

SEQ ID NO: 347 - RNA, 27 nt, Artificial Sequence
  Description of Artificial Sequence Synthetic oligonucleotide
ggccaagauccaagauaaagaaggcuu

SEQ ID NO: 348 - RNA, 27 nt, Artificial Sequence
  Description of Artificial Sequence Synthetic oligonucleotide
gccuucuuuaucuuggaucuuggccuu

SEQ ID NO: 349 - DNA, 120 bp, Homo sapiens
acgtgtgccgtgtgcggggacaacgccgcctgccagcactacggcgtgcgaacctgcgagggctgcaagggctttttcaagagaacagtgcagaaaaatgcaaaatatgtttgcctggca

SEQ ID NO: 350 - DNA, 120 bp, Homo sapiens
cgctgtgctgtgtgtggggacaacgcttcatgccagcattatggtgtccgcacatgtgagggctgcaagggcttcttcaagcgcacagtgcagaaaaacgccaagtacatctgcctggct

SEQ ID NO: 351 - DNA, 120 bp, Homo sapiens
ctgtgcgctgtgtgtggggacaacgcggcctgccaacactacggcgtgcgcacctgtgagggctgcaaaggcttctttaagcgcacagtgcaaaaaaatgcaaaatacgtgtgtttagca

SEQ ID NO: 352 - DNA, 19 bp, Artificial Sequence
  Description of Artificial Sequence Synthetic oligonucleotide
cgctgtgtgtggggacaac

SEQ ID NO: 353 - DNA, 19 bp, Artificial Sequence
  Description of Artificial Sequence Synthetic oligonucleotide
tgccgtgtgcggggacaac

SEQ ID NO: 354 - DNA, 19 bp, Artificial Sequence
  Description of Artificial Sequence Synthetic oligonucleotide
gggctgcaaaggcttcttt

SEQ ID NO: 355 - DNA, 19 bp, Artificial Sequence
  Description of Artificial Sequence Synthetic oligonucleotide
gggctgcaagggctttttc

SEQ ID NO: 356 - DNA, 19 bp, Artificial Sequence
  Description of Artificial Sequence Synthetic oligonucleotide
gcgcacagtgcaaaaaaat

SEQ ID NO: 357 - DNA, 19 bp, Artificial Sequence
  Description of Artificial Sequence Synthetic oligonucleotide
gagaacagtgcagaaaaat

SEQ ID NO: 358 - DNA, 50 bp, Homo sapiens
ctgcctcttcattgcctgcttcgtcctggtcctcacgcagagctccatct

SEQ ID NO: 359 - DNA, 49 bp, Homo sapiens
ctgcctcatggttgcctgtccggtcctcatcctcacccagagctccatc

SEQ ID NO: 360 - DNA, 50 bp, Homo sapiens
ccatgaactacatggtgtacttcaacttctttgcctgtgtgctggtgccc

SEQ ID NO: 361 - DNA, 47 bp, Homo sapiens
gcatggagtacatggtctacttcaacttctttgtgtgggtgctgccc

SEQ ID NO: 362 - DNA, 19 bp, Artificial Sequence
  Description of Artificial Sequence Synthetic oligonucleotide
cctcacccagagctccatc

SEQ ID NO: 363 - DNA, 19 bp, Artificial Sequence
  Description of Artificial Sequence Synthetic oligonucleotide
catggtctacttcaacttc

SEQ ID NO: 364 - DNA, 19 bp, Artificial Sequence
  Description of Artificial Sequence Synthetic oligonucleotide
gtctacttcaacttctttg

SEQ ID NO: 365 - DNA, 120 bp, Homo sapiens
aagctaattcatctggagatcaaacccgcaatccggaaccagatcataagggagctgcaggttctgcatgagtgcaactctccgtacatcgtgggcttctatggtgcgttctacagcgat

SEQ ID NO: 366 - DNA, 120 bp, Homo sapiens
aagctgatccaccttgagatcaagccggccatccggaaccagatcatccgcgagctgcaggtcctgcacgaatgcaactcgccgtacatcgtgggcttctacggggccttctacagtgac

SEQ ID NO: 367 - DNA, 180 bp, Homo sapiens
gcctgacatatctgagggagaagcacaagatcatgcacagagatgtcaagccctccaacatcctagtcaactcccgtggggagatcaagctctgtgactttggggtcagcgggcagctcatcgactccatggccaactccttcgtgggcacaaggtcctacatgtcgccagaaagactcc

SEQ ID NO: 368 - DNA, 180 bp, Homo sapiens
gcttggcgtacctccgagagaagcaccagatcatgcaccgagatgtgaagccctccaacatcctcgtgaactctagaggggagatcaagctgtgtgacttcggggtgagcggccagctcatcgactccatggccaactccttcgtgggcacgcgctcctacatggctccggagcggttgc

SEQ ID NO: 369 - DNA, 19 bp, Artificial Sequence
  Description of Artificial Sequence Synthetic oligonucleotide
catccggaaccagatcatc

SEQ ID NO: 370 - DNA, 19 bp, Artificial Sequence
  Description of Artificial Sequence Synthetic oligonucleotide
gtacatcgtgggcttctac

SEQ ID NO: 371 - DNA, 19 bp, Artificial Sequence
  Description of Artificial Sequence Synthetic oligonucleotide
gaagccctccaacatcctc

SEQ ID NO: 372 - DNA, 60 bp, Homo sapiens
ttgctgaaaagccagcagctgagcaatgaccatatctgctacttcctctaccagatcctg

SEQ ID NO: 373 - DNA, 60 bp, Homo sapiens
ctcttgaagacacaacacctcagcaatgaccatatctgctattttctctaccagatcctc

SEQ ID NO: 374 - DNA, 240 bp, Homo sapiens
gccccagagatcatgctgaactccaagggctataccaagtccatcgacatctggtctgtgggctgcattctggctgagatgctctctaaccggcccatcttccctggcaagcactacctggatcagctcaaccacattctgggcatcctgggctccccatcccaggaggacctgaattgtatcatcaacatgaaggcccgaaactacctacagtctctgccctccaagaccaaggtggct

SEQ ID NO: 375 - DNA, 240 bp, Homo sapiens
gctccagaaattatgttgaattccaagggctacaccaagtccattgatatttggtctgtaggctgcattctggcagaaatgctttctaacaggcccatctttccagggaagcattatcttgaccagctgaaccacattttgggtattcttggatccccatcacaagaagacctgaattgtataataaatttaaaagctaggaactatttgctttctcttccacacaaaaataaggtgcca

SEQ ID NO: 376 - DNA, 19 bp, Artificial Sequence
  Description of Artificial Sequence Synthetic oligonucleotide
tcagcaatgaccatatctg

SEQ ID NO: 377 - DNA, 19 bp, Artificial Sequence
  Description of Artificial Sequence Synthetic oligonucleotide
ccaagggctacaccaagtc

SEQ ID NO: 378 - DNA, 19 bp, Artificial Sequence
  Description of Artificial Sequence Synthetic oligonucleotide
gtctgtaggctgcattctg

SEQ ID NO: 379 - DNA, 19 bp, Artificial Sequence
  Description of Artificial Sequence Synthetic oligonucleotide
agaagacctgaattgtata

SEQ ID NO: 380 - DNA, 60 bp, Homo sapiens
cgggccctcgcgccggcttctaccggcaggagctgaacaagaccgtgtgggaggtgccgc

SEQ ID NO: 381 - DNA, 51 bp, Homo sapiens
ggcccacgttctaccggcaggagctgaacaagacaatctgggaggtgcccg

SEQ ID NO: 382 - DNA, 120 bp, Homo sapiens
cgaggagatgaccggctatgtggccacgcgctggtaccgggcacctgagatcatgctcaactggatgcattacaaccaaacagtggatatctggtccgtgggctgcatcatggctgagct

SEQ ID NO: 383 - DNA, 120 bp, Homo sapiens
tgatgaaatgacaggctacgtggccactaggtggtacagggctcctgagatcatgctgaactggatgcattacaaccagacagttgatatttggtcagtgggatgcataatggccgagct

SEQ ID NO: 384 - DNA, 54 bp, Homo sapiens
ttattttttttgagaccgagtcttgctctgttacccaggctggagtgcagtggc

SEQ ID NO: 385 - DNA, 60 bp, Homo sapiens
gttttgttttgttttttgagacagggtcttgctctgtcgcccaggctggagtgcactggc

SEQ ID NO: 386 - DNA, 19 bp, Artificial Sequence
  Description of Artificial Sequence Synthetic oligonucleotide
gacagggtcttgctctgtc

SEQ ID NO: 387 - DNA, 19 bp, Artificial Sequence
  Description of Artificial Sequence Synthetic oligonucleotide
cgcccaggctggagtgcac

SEQ ID NO: 388 - DNA, 60 bp, Homo sapiens
tcatcgtcatggtgggcctccccgcccggggcaagacctacatctccaagaagctgactc

SEQ ID NO: 389 - DNA, 60 bp, Homo sapiens
tcattgtcatggtgggcctgcccgccaggggcaagacctacatctccaagaagctgactc

SEQ ID NO: 390 - DNA, 60 bp, Homo sapiens
tggagcggcaggagaatgtgctggtcatctgccaccaggccgtcctgcgctgcctgcttg

SEQ ID NO: 391 - DNA, 60 bp, Homo sapiens
tggagaggcaagagaatgtgctggtcatctgccaccaggctgtgatgcgctgcctgctgg

SEQ ID NO: 392 - RNA, 45 nt, Artificial Sequence
  Description of Artificial Sequence Synthetic oligonucleotide
gcgacugguuacccggucgcaaaauuuaucuuggaucuuggccuu

SEQ ID NO: 393 - RNA, 45 nt, Artificial Sequence
  Description of Artificial Sequence Synthetic oligonucleotide
gcgacugguuacccggucgcaauuggccaagauccaagauaaauu

SEQ ID NO: 394 - RNA, 104 nt, Artificial Sequence
  Description of Artificial Sequence Synthetic polynucleotide
gaauuccgcgugugccagcgaaaguugcguaugggucacaucgcaggcacaugucaucugggcgguccguucgggauccaaaauuaucuuggaucuuggccuca

SEQ ID NO: 395 - RNA, 59 nt, Artificial Sequence
  Description of Artificial Sequence Synthetic oligonucleotide
agccgcgaggggagggauaggguagggcgcggcuaaaaaggccaagauccaagauaauu

SEQ ID NO: 396 - RNA, 47 nt, Artificial Sequence
  Description of Artificial Sequence Synthetic oligonucleotide
acguaucccuuuucgcguaagccuucuuuaucuuggaucuuggccuu

SEQ ID NO: 397 - RNA, 106 nt, Artificial Sequence
  Description of Artificial Sequence Synthetic polynucleotide
gaauuccgcgugugccagcgaaaguugcguaugggucacaucgcaggcacaugucaucugggcgguccguucgggauccuuggccaagauccaagauaaagaaggc

SEQ ID NO: 398 - RNA, 43 nt, Artificial Sequence
  Description of Artificial Sequence Synthetic oligonucleotide
acguaucccuuuucgcguaaaaucgaaguacucagcguaaguu

SEQ ID NO: 399 - RNA, 104 nt, Artificial Sequence
  Description of Artificial Sequence Synthetic polynucleotide
gaauuccgcgugugccagcgaaaguugcguaugggucacaucgcaggcacaugucaucugggcgguccguucgggauccuuuucuuacgcugaguacuucgauu

SEQ ID NO: 400 - RNA, 45 nt, Artificial Sequence
  Description of Artificial Sequence Synthetic oligonucleotide
acguaucccuuuucgcguuuggccaagauccaagauaaagaaggc

SEQ ID NO: 401 - RNA, 108 nt, Artificial Sequence
  Description of Artificial Sequence Synthetic polynucleotide
gaauuccgcgugugccagcgaaaguugcguaugggucacaucgcaggcacaugucaucugggcgguccguucgggauccaagccuucuuuaucuuggaucuuggccuu

SEQ ID NO: 402 - RNA, 43 nt, Artificial Sequence
  Description of Artificial Sequence Synthetic oligonucleotide
acguaucccuuuucgcguuuuucuuacgcugaguacuucgauu

SEQ ID NO: 403 - RNA, 64 nt, Artificial Sequence
  Description of Artificial Sequence Synthetic oligonucleotide
gggaggacgaugcggaucagccauguuuacgucacuccuaaaauguagagaugcggugguccuu

SEQ ID NO: 404 - RNA, 21 nt, Artificial Sequence
  Description of Artificial Sequence Synthetic oligonucleotide
uuaucuuggaucuuggccuuc

SEQ ID NO: 405 - RNA, 87 nt, Artificial Sequence
  Description of Artificial Sequence Synthetic oligonucleotide
gggaggacgaugcggaucagccauguuuacgucacuccuaaaaaggccaagauccaagauaauuuuggaccaccgcaucucuacauu

SEQ ID NO: 406 - RNA, 21 nt, Artificial Sequence
  Description of Artificial Sequence Synthetic oligonucleotide
cuuacgcugaguacuucgauu

SEQ ID NO: 407 - DNA, 27 bp, Artificial Sequence
  Description of Artificial Sequence Synthetic oligonucleotide
  Description of Combined DNA/RNA Molecule Synthetic oligonucleotide
ggccaagauccaagauaaagaaggctt

SEQ ID NO: 408 - RNA, 84 nt, Artificial Sequence
  Description of Artificial Sequence Synthetic oligonucleotide
gccuucuuuaucuuggaucuuggccuuaaaaaaaggagaugcguaggguggacugagugaugucugucaagaccucguccaaac

SEQ ID NO: 409 - RNA, 84 nt, Artificial Sequence
  Description of Artificial Sequence Synthetic oligonucleotide
gccuucuuuaucuuggaucuuggccuuaaaaaaaggaggguugcguugcaaguaacagaaaggaauuugaaaauuguggcaaac

SEQ ID NO: 410 - RNA, 84 nt, Artificial Sequence
  Description of Artificial Sequence Synthetic oligonucleotide
gccuucuuuaucuuggaucuuggccuuaaaaaaaggagacacaugugacaagaggcuaugauccugaaugcauccuuggcaaac

SEQ ID NO: 411 - RNA, 84 nt, Artificial Sequence
  Description of Artificial Sequence Synthetic oligonucleotide
gccuucuuuaucuuggaucuuggccuuaaaaaaaggaguagugcagcauugacugaaugucauacggcauaagcaucuacaaac

SEQ ID NO: 412 - RNA, 84 nt, Artificial Sequence
  Description of Artificial Sequence Synthetic oligonucleotide
gccuucuuuaucuuggaucuuggccuuaaaaaaaggaguauacauucuugguucauaaaggauaaggccuaagucgggucaaac

SEQ ID NO: 413 - RNA, 50 nt, Artificial Sequence
  Description of Artificial Sequence Synthetic oligonucleotide
ggagaugcguaggguggacugagugaugucugucaagaccucguccaaac

SEQ ID NO: 414 - RNA, 50 nt, Artificial Sequence
  Description of Artificial Sequence Synthetic oligonucleotide
ggaggguugcguugcaaguaacagaaaggaauuugaaaauuguggcaaac

SEQ ID NO: 415 - RNA, 50 nt, Artificial Sequence
  Description of Artificial Sequence Synthetic oligonucleotide
ggagacacaugugacaagaggcuaugauccugaaugcauccuuggcaaac

SEQ ID NO: 416 - RNA, 50 nt, Artificial Sequence
  Description of Artificial Sequence Synthetic oligonucleotide
ggaguagugcagcauugacugaaugucauacggcauaagcaucuacaaac

SEQ ID NO: 417 - RNA, 50 nt, Artificial Sequence
  Description of Artificial Sequence Synthetic oligonucleotide
ggaguauacauucuugguucauaaaggauaaggccuaagucgggucaaac

SEQ ID NO: 418 - DNA, 89 bp, Artificial Sequence
  Description of Artificial Sequence Synthetic oligonucleotide
gggagacaaacaaagagcgacaagggcagagtggacggtcggctagagccgggaggaattccttgtgaccacccagaagaagccagaag

SEQ ID NO: 419 - RNA, 13 nt, Artificial Sequence
  Description of Artificial Sequence Synthetic oligonucleotide
uugauuuacaaac

SEQ ID NO: 420 - RNA, 13 nt, Artificial Sequence
  Description of Artificial Sequence Synthetic oligonucleotide
uugauuuacuaac

SEQ ID NO: 421 - DNA, 19 bp, Artificial Sequence
  Description of Artificial Sequence Synthetic oligonucleotide
gaactggatgcattacaac

SEQ ID NO: 422 - DNA, 19 bp, Mus musculus
ggcaaagatccaggacaag